SEQUENCE LISTING (65 sequences)

SEQ ID NO: 1 - DNA, 933 bp, Alicyclobacillus acidocaldarius
atgccgctcgatcccgtcattcagcaggtgctcgatcaactcaaccgcatgcctgccccggactacaaacatctctccgcccagcaatttcgttcccaacagtcgctgtttcctcctgtcaagaaggagcccgtggccgaggtccgagagtttgacatggatctgcctggccgcacgctcaaggtgcgcatgtaccgcccggagggcgtcgaaccgccctaccccgcgctcgtgtattatcacggcggcggttgggtcgtcggagacctcgagacgcacgatcccgtctgccgcgtcctcgcgaaagacggccgcgcggtcgtgttctccgtcgactaccgcctggcgccggagcacaagttccctgccgccgtggaagacgcctacgacgcgcttcagtggatcgcggagcgcgcagcggactttcatctcgatccagcccgcatcgcggtcggcggagacagcgccggagggaatcttgccgctgtgacgagcatccttgccaaagagcgcggcgggccggccatcgcgttccagctgctcatctacccttccacggggtacgatccggctcatcctcccgcatctatcgaagaaaatgcggaaggctatctcctgaccggcggcatgatgctctggttccgggatcaatacttgaacagcctggaggaactcacgcatccgtggttttcacccgtcctctacccggacttgagcggcttgcctccggcgtacatcgcgacggcgcagtacgatccgctgcgcgacgtcggcaagctttacgcggaagcgctgaacaaggcgggcgtcaaggtcgagatcgagaacttcgaagatctgatccacggattcgcacagttttacagcctttcgcctggcgcgacgaaggcgctcgtccgcattgcggagaaacttcgagacgcgctggcctga

SEQ ID NO: 2 - protein, 310 aa, Alicyclobacillus acidocaldarius
  MOD_RES (53)..(53): Any amino acid
MPLDPVIQQVLDQLNRMPAPDYKHLSAQQFRSQQSLFPPVKKEPVAEVREFDXDLPGRTLKVRXYRPEGVEPPYPALVYYHGGGWVVGDLETHDPVCRVLAKDGRAVVFSVDYRLAPEHKFPAAVEDAYDALQWIAERAADFHLDPARIAVGGDSAGGNLAAVTSILAKERGGPALAFQLLIYPSTGYDPAHPPASIEENAEGYLLTGGXXLWFRDQYLNSLEELTHPWFSPVLYPDLSGLPPAYIATAQYDPLRDVGKLYAEALNKAGVKVEIENFEDLIHGFAQFYSLSPGATKALVRIAEKLRDALA

SEQ ID NO: 3 - DNA, 1065 bp, Thermus thermophilus
atgctggacaagcttgaccgcctagaggaagagtaccgggagctggaggcgctcctctccgacccggaggtgctgaaggacaaggggcgctaccagagcctctcccgccgctacgccgagatgggggaggtgatcggcctcatccgggagtaccggaaggtgctggaggacctggagcaggcggaaagccttcttgacgaccccgagctcaaggagatggccaaggcggagcgggaggccctcctcgcccgcaaggaggccctggagaaggagctggagcgccacctcctgcctaaggaccccatggacgaaagggacgccatcgtagagatccgggcggggacgggaggggaggaggccgccctcttcgcccgcgaccttttcaacatgtacctccgcttcgccgaggagatgggctttgagacggaggtcctggactcccaccccacggacctcgggggcttctccaaggtggtctttgaggtgcggggcccgggggcctacggcaccttcaagtacgagagcggggtccaccgggtgcaacgggtgcccgtcaccgagacccaggggcggatccacacctccaccgccacggtggccgtcctccccaaggcggaggaggaggacttcgccctcaacatggacgagatccgcattgacgtgatgcgggcctcggggcccggggggcagggggtgaacaccacggactcggcggtgcgggtggtccacctgcccacggggatcatggtcacctgccaggactcccgcagccagatcaagaaccgggagaaggccctcatgatcctaagaagccgtctcctggagatgaagcgggcggaggaggcggaaaggctccggaagacccgccttgcccagatcggcaccggggagcgctcggagaagatccgcacctacaacttcccccagtcccgggtcacggaccaccgcatcgggttcaccacccacgacctcgagggcgtcctctccggccacctgacccccatcctggaggcgctcaagcgggccgaccaggagcgccagctcgcggcgctggcggaagggtga

SEQ ID NO: 4 - protein, 354 aa, Thermus thermophilus
MLDKLDRLEEEYRELEALLSDPEVLKDKGRYQSLSRRYAEMGEVIGLIREYRKVLEDLEQAESLLDDPELKEMAKAEREALLARKEALEKELERHLLPKDPMDERDAIVEIRAGTGGEEAALFARDLFNMYLRFAEEMGFETEVLDSHPTDLGGFSKVVFEVRGPGAYGTFKYESGVHRVQRVPVTETQGRIHTSTATVAVLPKAEEEDFALNMDEIRIDVMRASGPGGQGVNTTDSAVRVVHLPTGIMVTCQDSRSQIKNREKALMILRSRLLEMKRAEEAERLRKTRLAQIGTGERSEKIRTYNFPQSRVTDHRIGFTTHDLEGVLSGHLTPILEALKRADQERQLAALAEG

SEQ ID NO: 5 - DNA, 1083 bp, Escherichia coli
atgaagccttctatcgttgccaaactggaagccctgcatgaacgccatgaagaagttcaggcgttgctgggtgacgcgcaaactatcgccgaccaggaacgttttcgcgcattatcacgcgaatatgcgcagttaagtgatgtttcgcgctgttttaccgactggcaacaggttcaggaagatatcgaaaccgcacagatgatgctcgatgatcctgaaatgcgtgagatggcgcaggatgaactgcgcgaagctaaagaaaaaagcgagcaactggaacagcaattacaggttctgttactgccaaaagatcctgatgacgaacgtaacgccttcctcgaagtccgagccggaaccggcggcgacgaagcggcgctgttcgcgggcgatctgttccgtatgtacagccgttatgccgaagcccgccgctggcgggtagaaatcatgagcgccagcgagggtgaacatggtggttataaagagatcatcgccaaaattagcggtgatggtgtgtatggtcgtctgaaatttgaatccggcggtcatcgcgtgcaacgtgttcctgctacggaatcgcagggtcgtattcatacttctgcttgtaccgttgcggtaatgccagaactgcctgacgcagaactgccggacatcaacccagcagatttacgcattgatactttccgctcgtcaggggcgggtggtcagcacgttaacaccaccggttcggcaattcgtattactcacttgccgaccgggattgttgttgaatgtcaggacgaacgttcacaacataaaaacaaagctaaagcactttctgttctcggtgctcgcatccacgctgctgaaatggcaaaacgccaacaggccgaagcgtctacccgtcgtaacctgctggggagtggcgatcgcagcgaccgtaaccgtacttacaacttcccgcaggggcgcgttaccgatcaccgcatcaacctgacgctctaccgcctggatgaagtgatggaaggtaagctggatatgctgattgaaccgattatccaggaacatcaggccgaccaactggcggcgttgtccgagcaggaataa

SEQ ID NO: 6 - protein, 360 aa, Escherichia coli
MKPSIVAKLEALHERHEEVQALLGDAQTIADQERFRALSREYAQLSDVSRCFTDWQQVQEDIETAQMMLDDPEMREMAQDELREAKEKSEQLEQQLQVLLLPKDPDDERNAFLEVRAGTGGDEAALFAGDLFRMYSRYAEARRWRVEIMSASEGEHGGYKEIIAKISGDGVYGRLKFESGGHRVQRVPATESQGRIHTSACTVAVMPELPDAELPDINPADLRIDTFRSSGAGGQHVNTTDSAIRITHLPTGIVVECQDERSQHKNKAKALSVLGARIHAAEMAKRQQAEASTRRNLLGSGDRSDRNRTYNFPQGRVTDHRINLTLYRLDEVMEGKLDMLIEPIIQEHQADQLAALSEQE

SEQ ID NO: 7 - DNA, 39 bp, Artificial Sequence
  Description of Artificial Sequence Synthetic oligonucleotide
gagctcggtaccattgagggtcgcggttccggcggtggt

SEQ ID NO: 8 - DNA, 5156 bp, Artificial Sequence
  Description of Artificial Sequence Synthetic polynucleotide
cggtaccattgagggtcgcggttccggcggtggtatggcgctcgatcccgtcattcagcaggtgctcgatcaactcaaccgcatgcctgccccggactacaaacatctctccgcccagcaatttcgttcccaacagtcgctgtttcctcctgtcaagaaggagcccgtggccgaggtccgagagtttgacatggatctgcctggccgcacgctcaaggtgcgcatgtaccgcccggagggcgtcgaaccgccctaccccgcgctcgtgtattatcacggcggcggttgggtcgtcggagacctcgagacgcacgatcccgtctgccgcgtcctcgcgaaagacggccgcgcggtcgtgttctccgtcgactaccgcctggcgccggagcacaagttccctgccgccgtggaagacgcctacgacgcgcttcagtggatcgcggagcgcgcagcggactttcatctcgatccagcccgcatcgcggtcggcggagacagcgccggagggaatcttgccgctgtgacgagcatccttgccaaagagcgcggcgggccggccatcgcgttccagctgctcatctacccttccacggggtacgatccggctcatcctcccgcatctatcgaagaaaatgcggaaggctatctcctgaccggcggcatgatgctctggttccgggatcaatacttgaacagcctggaggaactcacgcatccgtggttttcacccgtcctctacccggacttgagcggcttgcctccggcgtacatcgcgacggcgcagtacgatccgctgcgcgacgtcggcaagctttacgcggaagcgctgaacaaggcgggcgtcaaggtcgagatcgagaacttcgaagatctgatccacggattcgcacagttttacagcctttcgcctggcgcgacgaaggcgctcgtccgcattgcggagaaacttcgagacgcgctggcctgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaaggcggccgcgtcgactcgagcgagct

SEQ ID NO: 9 - DNA, 4460 bp, Artificial Sequence
  Description of Artificial Sequence Synthetic polynucleotide
catggcgctcgatcccgtcattcagcaggtgctcgatcaactcaaccgcatgcctgccccggactacaaacatctctccgcccagcaatttcgttcccaacagtcgctgtttcctcctgtcaagaaggagcccgtggccgaggtccgagagtttgacatggatctgcctggccgcacgctcaaggtgcgcatgtaccgcccggagggcgtcgaaccgccctaccccgcgctcgtgtattatcacggcggcggttgggtcgtcggagacctcgagacgcacgatcccgtctgccgcgtcctcgcgaaagacggccgcgcggtcgtgttctccgtcgactaccgcctggcgccggagcacaagttccctgccgccgtggaagacgcctacgacgcgcttcagtggatcgcggagcgcgcagcggactttcatctcgatccagcccgcatcgcggtcggcggagacagcgccggagggaatcttgccgctgtgacgagcatccttgccaaagagcgcggcgggccggccatcgcgttccagctgctcatctacccttccacggggtacgatccggctcatcctcccgcatctatcgaagaaaatgcggaaggctatctcctgaccggcggcatgatgctctggttccgggatcaatacttgaacagcctggaggaactcacgcatccgtggttttaccccgtcctctacccggacttgagcggcttgcctccggcgtacatcgcgacggcgcagtacgatccgctgcgcgacgtcggcaagctttacgcggaagcgctgaacaaggcgggcgtcaaggtcgagatcgagaacttcgaagatctgatcctcggattcgcacagttttacagcctttcgcctggcgcgacgaaggcgctcgtccgcattgcggagaaacttcgagacgcgctggcctgagagctcccggggggggttctcatcatcatcatcatcattaataaaagggcgaattccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatac

SEQ ID NO: 10 - DNA, 4460 bp, Artificial Sequence
  Description of Artificial Sequence Synthetic polynucleotide
catggcgctcgatcccgtcattcagcaggtgctcgatcaactcaaccgcatgcctgccccggactacaaacatctctccgcccagcaatttcgttcccaacagtcgctgtttcctcctgtcaagaaggagcccgtggccgaggtccgagagtttgacatggatctgcctggccgcacgctcaaggtgcgcatgtaccgcccggagggcgtcgaaccgccctaccccgcgctcgtgtattatcacggcggcggttgggtcgtcggagacctcgagacgcacgatcccgtctgccgcgtcctcgcgaaagacggccgcgcggtcgtgttctccgtcgactaccgcctggcgccggagcacaagttccctgccgccgtggaagacgcctacgacgcgcttcagtggatcgcggagcgcgcagcggactttcatctcgatccagcccgcatcgcggtcggcggagactaggccggagggaatcttgccgctgtgacgagcatccttgccaaagagcgcggcgggccggccatcgcgttccagctgctcatctacccttccacggggtacgatccggctcatcctcccgcatctatcgaagaaaatgcggaaggctatctcctgaccggcggcatgatgctctggttccgggatcaatacttgaacagcctggaggaactcacgcatccgtggttttaccccgtcctctacccggacttgagcggcttgcctccggcgtacatcgcgacggcgcagtacgatccgctgcgcgacgtcggcaagctttacgcggaagcgctgaacaaggcgggcgtcaaggtcgagatcgagaacttcgaagatctgatcctcggattcgcacagttttacagcctttcgcctggcgcgacgaaggcgctcgtccgcattgcggagaaacttcgagacgcgctggcctgagagctcccggggggggttctcatcatcatcatcatcattaataaaagggcgaattccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatac

SEQ ID NO: 11 - DNA, 5042 bp, Artificial Sequence
  Description of Artificial Sequence Synthetic polynucleotide
tatggaggcgacccttcccgttttggacgcgaagacggcggccctaaagaggcgttccatccggcgttaccggaaggaccccgtacccgaggggcttctccgggaaatcctcgaggccgccctccgggcgccctcggcctggaacctccagccctggcggatcgtggtggtgcgggaccccgccaccaaacgggccctgagggaggcggccttcggccaggcccacgtggaggaggcccccgtggtcctggtcctctacgccgacctcgaggacgctctcgcccacctggacgaggtcatccaccccggggtccagggggaaaggcgtgaggcgcagaagcaggccatccaacgggccttcgccgccatggggcaagaggcgcgaaaggcctgggcctccgggcagagctacatcctcttgggctacctccttctcctcctggaggcttatggcctcggaagcgtccccatgctggggtttgaccccgagagggtgagggcgatcctggggcttccttcccgcgccgccatccccgccctggtggccttgggctacccggcggaggagggctacccctcccaccgcctgcccctggagcgggtggtcctctggcgcgagctcggtaccattgagggtcgcggttccggcggtggtatggcgctcgatcccgtcattcagcaggtgctcgatcaactcaaccgcatgcctgccccggactacaaacatctctccgcccagcaatttcgttcccaacagtcgctgtttcctcctgtcaagaaggagcccgtggccgaggtccgagagtttgacatggatctgcctggccgcacgctcaaggtgcgcatgtaccgcccggagggcgtcgaaccgccctaccccgcgctcgtgtattatcacggcggcggttgggtcgtcggagacctcgagacgcacgatcccgtctgccgcgtcctcgcgaaagacggccgcgcggtcgtgttctccgtcgactaccgcctggcgccggagcacaagttccctgccgccgtggaagacgcctacgacgcgcttcagtggatcgcggagcgcgcagcggactttcatctcgatccagcccgcatcgcggtcggcggagacagcgccggagggaatcttgccgctgtgacgagcatccttgccaaagagcgcggcgggccggccatcgcgttccagctgctcatctacccttccacggggtacgatccggctcatcctcccgcatctatcgaagaaaatgcggaaggctatctcctgaccggcggcatgatgctctggttccgggatcaatacttgaacagcctggaggaactcacgcatccgtggttttcacccgtcctctacccggacttgagcggcttgcctccggcgtacatcgcgacggcgcagtacgatccgctgcgcgacgtcggcaagctttacgcggaagcgctgaacaaggcgggcgtcaaggtcgagatcgagaacttcgaagatctgatccacggattcgcacagttttacagcctttcgcctggcgcgacgaaggcgctcgtccgcattgcggagaaacttcgagacgcgctggcctgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatataccatggcaca

SEQ ID NO: 12 - DNA, 5633 bp, Artificial Sequence
  Description of Artificial Sequence Synthetic polynucleotide
catggcgaagggcgagtttgttcggacgaagcctcacgtgaacgtggggacgattgggcacgtggaccacgggaagacgacgctgacggcggcgttaacgtatgtggcggcggcggagaacccgaatgtagaggttaaggactacggggacattgacaaggcgccggaggagcgtgcgcgggggattacgatcaacacggcgcacgtggagtacgagacggcgaagcggcactattcccacgtggattgccctgggcacgcggactacatcaagaacatgatcacgggtgccgcgcagatggacggggcgatccttgtggtgtcggcggcggacgggccgatgccgcagacgcgggagcacattttgctggcgcggcaggtgggggtgccgtacattgtggtgttcatgaacaaggtggacatggtggacgaccccgagttgctggacctggtggagatggaggtgcgggaccttttgaaccagtacgagtttcctggggacgaggttccggtgattcgggggagtgctcttttggcgcttgagcagatgcacaggaacccgaagacgaggcgtggggagaacgagtgggtggacaagatttgggagctgttggacgcgattgacgagtacattcccacgccggtgcgggacgtggacaagccgttcttgatgccggtggaggacgtgtttacgatcacgggtcgtgggacggtggccacgggtcggattgagcggggcaaggtgaaggttggggacgaggtggagattgtgggccttgctccggagacgcggaggacggtggtgacgggtgtggagatgcaccggaagaccttgcaggaggggattgctggggacaatgtgggggtgctcctgcggggtgtgagccgggaggaggtggagcgggggcaggtgctggcgaagcctgggagcattacgccgcacacgaagtttgaggcctcggtgtatgtgttgaagaaggaggagggtggacggcacacggggtttttttcggggtaccgtccgcagttttactttcggacgacggacgtgacgggggtggtgcagttgcctccgggcgtggagatggtgatgcctggggacaacgtgacgtttacggtggagctgatcaagccggtgggcctggaggagggtttgcggtttgccatccgtgagggtgggcggaccgtgggcgccggcgtcgtcaccaagatcctggagctcggtaccattgagggtcgcggttccggcggtggtatggcgctcgatcccgtcattcagcaggtgctcgatcaactcaaccgcatgcctgccccggactacaaacatctctccgcccagcaatttcgttcccaacagtcgctgtttcctcctgtcaagaaggagcccgtggccgaggtccgagagtttgacatggatctgcctggccgcacgctcaaggtgcgcatgtaccgcccggagggcgtcgaaccgccctaccccgcgctcgtgtattatcacggcggcggttgggtcgtcggagacctcgagacgcacgatcccgtctgccgcgtcctcgcgaaagacggccgcgcggtcgtgttctccgtcgactaccgcctggcgccggagcacaagttccctgccgccgtggaagacgcctacgacgcgcttcagtggatcgcggagcgcgcagcggactttcatctcgatccagcccgcatcgcggtcggcggagacagcgccggagggaatcttgccgctgtgacgagcatccttgccaaagagcgcggcgggccggccatcgcgttccagctgctcatctacccttccacggggtacgatccggctcatcctcccgcatctatcgaagaaaatgcggaaggctatctcctgaccggcggcatgatgctctggttccgggatcaatacttgaacagcctggaggaactcacgcatccgtggttttcacccgtcctctacccggacttgagcggcttgcctccggcgtacatcgcgacggcgcagtacgatccgctgcgcgacgtcggcaagctttacgcggaagcgctgaacaaggcgggcgtcaaggtcgagatcgagaacttcgaagatctgatccacggattcgcacagttttacagcctttcgcctggcgcgacgaaggcgctcgtccgcattgcggagaaacttcgagacgcgctggcctgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatac

SEQ ID NO: 13 - DNA, 5009 bp, Artificial Sequence
  Description of Artificial Sequence Synthetic polynucleotide
tatgagccaaatggaactcatcaagaagctacgcgaagccacgggggccgggatgatggacgtgaagcgggccctcgaggacgccggctgggacgaggagaaggcggtccagctcctccgggagcggggggcgatgaaggccgccaagaaggcggaccgggaagcccgcgagggcatcatcggccactacatccaccacaaccagcgggtgggggtcctggtggagctcaactgcgaaacggacttcgtggcccggaacgagctcttccagaacctggccaaggacctcgccatgcacatcgccatgatgaacccccgctacgtctccgccgaggagatccccgccgaggagctggaaaaagagcggcagatctacattcaggccgccctgaacgaggggaagccccagcagatcgccgagaagatcgccgaaggccgcctcaagaagtacctggaggaggtggtcctgctggagcagcccttcgtcaaggacgacaaggtcaaggtgaaggagctcatccagcaggccatcgccaagatcggcgagaacatcgtggtccgacgcttctgccgctttgagctgggggcgggtaccattgagggtcgcggttccggcggtggtatggcgctcgatcccgtcattcagcaggtgctcgatcaactcaaccgcatgcctgccccggactacaaacatctctccgcccagcaatttcgttcccaacagtcgctgtttcctcctgtcaagaaggagcccgtggccgaggtccgagagtttgacatggatctgcctggccgcacgctcaaggtgcgcatgtaccgcccggagggcgtcgaaccgccctaccccgcgctcgtgtattatcacggcggcggttgggtcgtcggagacctcgagacgcacgatcccgtctgccgcgtcctcgcgaaagacggccgcgcggtcgtgttctccgtcgactaccgcctggcgccggagcacaagttccctgccgccgtggaagacgcctacgacgcgcttcagtggatcgcggagcgcgcagcggactttcatctcgatccagcccgcatcgcggtcggcggagacagcgccggagggaatcttgccgctgtgacgagcatccttgccaaagagcgcggcgggccggccatcgcgttccagctgctcatctacccttccacggggtacgatccggctcatcctcccgcatctatcgaagaaaatgcggaaggctatctcctgaccggcggcatgatgctctggttccgggatcaatacttgaacagcctggaggaactcacgcatccgtggttttcacccgtcctctacccggacttgagcggcttgcctccggcgtacatcgcgacggcgcagtacgatccgctgcgcgacgtcggcaagctttacgcggaagcgctgaacaaggcgggcgtcaaggtcgagatcgagaacttcgaagatctgatccacggattcgcacagttttacagcctttcgcctggcgcgacgaaggcgctcgtccgcattgcggagaaacttcgagacgcgctggcctgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatataccatggcaca

SEQ ID NO: 14 - DNA, 7304 bp, Artificial Sequence
  Description of Artificial Sequence Synthetic polynucleotide
catggatgaacaggctctattagggctaaatccaaatgctgattcagactttagacaaagggccctggcctattttgagcagttaaaaatttccccagatgcctggcaggtgtgtgcagaagctctagcccagaggacatacagtgatgatcatgtgaagtttttctgctttcaagtactggaacatcaagttaaatacaaatactcagaactaaccactgttcaacaacagctaattagggagacgctcatatcatggctgcaagctcagatgctgaatccccaaccagagaagacctttatacgaaataaagccgcccaagtcttcgccttgctttttgttacagagtatctcactaagtggcccaagtttttttttgacattctctcagtagtggacctaaatccaaggggagtagatctctacctgcgaatcctcatggctattgattcagagttggtggatcgtgatgtggtgcatacatcagaggaggctcgtaggaatactctcataaaagataccatgagggaacagtgcattccaaatctggtggaatcatggtaccaaatattacaaaattatcagtttactaattctgaagtgacgtgtcagtgccttgaagtagttggggcttatgtctcttggatagacttatcccttatagccaatgataggtttataaatatgctgctaggtcatatgtcaatagaagttctacgggaagaagcatgtgactgtttatttgaagttgtaaataaaggaatggaccctgttgataaaatgaaactagtggaatctttgtgtcaagtattacagtctgctgggtttttcagcattgaccaggaagaagatgttgacttcctggccagattttctaagttggtaaatggaatgggacagtcattgatagttagttggagtaaattaattaagaatggggatattaagaatgctcaagaggcactacaagctattgaaacaaaagtggcactgatgttgcagctactaattcatgaggatgatgatatttcttctaatattattggattttgttacgattatcttcatattttgaaacagcttacagtgctctcggatcagcaaaaagctaatgtagaggcaatcatgttggccgttatgaaaaaattgacttacgatgaagaatataactttgaaaatgagggtgaagatgaagccatgtttgtagaatatagaaaacaactgaagttactgttggacaggcttgctcaagtttcaccagagttactactggcctctgttcgcagagtttttagttctacactgcagaattggcagactacacggtttatggaagttgaagtagcaataagattgctgtatatgttggcagaagctcttccagtatctcatggtgctcacttctcaggtgatgtttcaaaagctagtgctttgcaggatatgatgcgaactctggtaacatcaggagtcagttcctatcagcatacatctgtgacattggagttcttcgaaactgttgttagatatgaaaagtttttcacagttgaacctcagcacattccatgtgtactaatggctttcttagatcacagaggtctgcggcattccagtgcaaaagttcggagcaggacggcttacctgttttctagatttgtcaaatctctcaataagcaaatgaatcctttcattgaggatattttgaatagaatacaagatttattagagctttctccacctgagaatggccaccagtccttactgagcagcgatgatcaactttttatttatgagacagctggagtgctgattgttaatagtgaatatccggcagaaaggaaacaagccttaatgaggaatctgttgactccactaatggagaagtttaaaattctgttagaaaagttgatgctggcacaagatgaagaaaggcaagcctctctagcagactgtcttaaccatgctgttggatttgcaagtcgaaccagtaaagctttcagcaacaaacagactgtgaaacaatgtggctgttccgaagtttatctggactgtttacagacattcttgccagccctcagttgtcccttacaaaaggatattctcagaagtggagtccgtactttccttcatcgaatgattatttgcctggaggaagaagttcttccgttcattccatctgcttcagaacatatgctcaaagattgtgaagcaaaagatctccaggagttcattcctcttatcaaccagattacggccaaattcaagatacaggtatccccgtttttacaacagatgttcatgcccctgcttcatgcaatttttgaagtgctgctccggccagcagaagaaaatgaccagtctgctgctttagagaagcagatgttgcggaggagttactttgctttcctgcaaacagtcacaggcagtgggatgagcgaagttatagcaaatcaaggtgcagagaatgtagaaagagtgttggttactgttatccaaggagcagttgaatatccagatccaattgcacagaaaacatgttttatcatcctctcaaagttggtagaactctggggaggtaaagatggaccagtgggatttgctgattttgtttataagcacattgtccccgcatgtttcctagcacctttaaaacaaacctttgacctggcagatgcacaaacagtattggctttatctgagtgtgcagtgacactgaaaacaattcatctcaaacggggcccagaatgtgttcagtatcttcaacaagaatacctgccctccttgcaagtagctccagaaataattcaggagttttgtcaagcgcttcagcagcctgatgctaaagtttttaaaaattacttaaaggtgttcttccagagagcaaagcccgagctcggtaccattgagggtcgcggttccggcggtggtatggcgctcgatcccgtcattcagcaggtgctcgatcaactcaaccgcatgcctgccccggactacaaacatctctccgcccagcaatttcgttcccaacagtcgctgtttcctcctgtcaagaaggagcccgtggccgaggtccgagagtttgacatggatctgcctggccgcacgctcaaggtgcgcatgtaccgcccggagggcgtcgaaccgccctaccccgcgctcgtgtattatcacggcggcggttgggtcgtcggagacctcgagacgcacgatcccgtctgccgcgtcctcgcgaaagacggccgcgcggtcgtgttctccgtcgactaccgcctggcgccggagcacaagttccctgccgccgtggaagacgcctacgacgcgcttcagtggatcgcggagcgcgcagcggactttcatctcgatccagcccgcatcgcggtcggcggagacagcgccggagggaatcttgccgctgtgacgagcatccttgccaaagagcgcggcgggccggccatcgcgttccagctgctcatctacccttccacggggtacgatccggctcatcctcccgcatctatcgaagaaaatgcggaaggctatctcctgaccggcggcatgatgctctggttccgggatcaatacttgaacagcctggaggaactcacgcatccgtggttttcacccgtcctctacccggacttgagcggcttgcctccggcgtacatcgcgacggcgcagtacgatccgctgcgcgacgtcggcaagctttacgcggaagcgctgaacaaggcgggcgtcaaggtcgagatcgagaacttcgaagatctgatccacggattcgcacagttttacagcctttcgcctggcgcgacgaaggcgctcgtccgcattgcggagaaacttcgagacgcgctggcctgaggatccggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatac

SEQ ID NO: 15 - DNA, 7025 bp, Artificial Sequence
  Description of Artificial Sequence Synthetic polynucleotide
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatcctcaggccagcgcgtctcgaagtttctccgcaatgcggacgagcgccttcgtcgcgccaggcgaaaggctgtaaaactgtgcgaatccgtggatcagatcttcgaagttctcgatctcgaccttgacgcccgccttgttcagcgcttccgcgtaaagcttgccgacgtcgcgcagcggatcgtactgcgccgtcgcgatgtacgccggaggcaagccgctcaagtccgggtagaggacgggtgaaaaccacggatgcgtgagttcctccaggctgttcaagtattgatcccggaaccagagcatcatgccgccggtcaggagatagccttccgcattttcttcgatagatgcgggaggatgagccggatcgtaccccgtggaagggtagatgagcagctggaacgcgatggccggcccgccgcgctctttggcaaggatgctcgtcacagcggcaagattccctccggcgctgtctccgccgaccgcgatgcgggctggatcgagatgaaagtccgctgcgcgctccgcgatccactgaagcgcgtcgtaggcgtcttccacggcggcagggaacttgtgctccggcgccaggcggtagtcgacggagaacacgaccgcgcggccgtctttcgcgaggacgcggcagacgggatcgtgcgtctcgaggtctccgacgacccaaccgccgccgtgataatacacgagcgcggggtagggcggttcgacgccctccgggcggtacatgcgcaccttgagcgtgcggccaggcagatccatgtcaaactctcggacctcggccacgggctccttcttgacaggaggaaacagcgactgttgggaacgaaattgctgggcggagagatgtttgtagtccggggcaggcatgcggttgagttgatcgagcacctgctgaatgacgggatcgagcgccataccaccgccggaaccgcgaccctcaatggtaccgagctctagagtggaacctccatttaactcaccctccaatatacgaacaaatccggttttacttccattatttcgaactaaactactatctaaattatcccccaacattattggggctaaaaaaagattatacaactttaaatatttatcactcttcttagaaaacgcatacagcatatcatggtagtcatcaaaagtgtaatccctaacttttaagctcttggaaatcacacctcttaggttttgcaacataatactcccgtacttattaagctttatcatcctttcatcaaaattgtctggaaacttattcactagataatcagatatcaccctaattgcgttcaagtgatttagaacagctaaagtcaacaaagacttaacctcctcagctttctcgacgtcaataggttcataatcataatagaaaaatagagccgaaccgagttcatgatatatgaaagagaagttactctttagaggattacaattatcgtcgaactgattctggtaatactcgccggcttttcccatatcgtgaaggacaacgacgtccttaaccatttccttaacaccgtttagatcaagaactattccatatctctccaatcttctcgagataatcttataataagactcacttattttaccatctaaaaccctataagaaccaatagcgtgatcaattaaaccttgtttctcataagcgcaaggcttgatcaacatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 16 - DNA, 27 bp, Artificial Sequence
  Description of Artificial Sequence Synthetic oligonucleotide
ccatggcgctcgatcccgtcattcagc

SEQ ID NO: 17 - DNA, 24 bp, Artificial Sequence
  Description of Artificial Sequence Synthetic oligonucleotide
gagctcctaggccagcgcgtctcg

SEQ ID NO: 18 - DNA, 18 bp, Artificial Sequence
  Description of Artificial Sequence Synthetic primer
ccatggtgagcaagggcg

SEQ ID NO: 19 - DNA, 27 bp, Artificial Sequence
  Description of Artificial Sequence Synthetic primer
gcggccgcctttgtacagctcgtccat

SEQ ID NO: 20 - DNA, 54 bp, Artificial Sequence
  Description of Artificial Sequence Synthetic primer
gagctcggtaccattgagggtcgcggttccggcggtggtatggcgctcgatccc

SEQ ID NO: 21 - DNA, 18 bp, Artificial Sequence
  Description of Artificial Sequence Synthetic primer
ggatcctcaggccagcgc

SEQ ID NO: 22 - DNA, 20 bp, Artificial Sequence
  Description of Artificial Sequence Synthetic primer
taatacgactcactataggg

SEQ ID NO: 23 - DNA, 35 bp, Artificial Sequence
  Description of Artificial Sequence Synthetic primer
attccctccggcctagtctccgccgaccgcgatgc

SEQ ID NO: 24 - DNA, 35 bp, Artificial Sequence
  Description of Artificial Sequence Synthetic primer
cggtcggcggagactaggccggagggaatcttgcc

SEQ ID NO: 25 - DNA, 20 bp, Artificial Sequence
  Description of Artificial Sequence Synthetic primer
ctagttattgctcagcggtg

SEQ ID NO: 26 - DNA, 35 bp, Artificial Sequence
  Description of Artificial Sequence Synthetic primer
ggaattctaatacgactcactataggagagatgcc

SEQ ID NO: 27 - DNA, 35 bp, Artificial Sequence
  Description of Artificial Sequence Synthetic primer
gtccgttcagccgctccggcatctctcctatagtg

SEQ ID NO: 28 - DNA, 35 bp, Artificial Sequence
  Description of Artificial Sequence Synthetic primer
ctccggttttagagaccggtccgttcagccgctcc

SEQ ID NO: 29 - DNA, 35 bp, Artificial Sequence
  Description of Artificial Sequence Synthetic primer
ccggtagagttgcccctactccggttttagagacc

SEQ ID NO: 30 - DNA, 35 bp, Artificial Sequence
  Description of Artificial Sequence Synthetic primer
gagagggggatttgaacccccggtagagttgcccc

SEQ ID NO: 31 - DNA, 42 bp, Artificial Sequence
  Description of Artificial Sequence Synthetic primer
aagcttggatggatcacctggcggagagagggggatttgaac

SEQ ID NO: 32 - DNA, 18 bp, Artificial Sequence
  Description of Artificial Sequence Synthetic primer
caggaaacagctatgacc

SEQ ID NO: 33 - DNA, 27 bp, Artificial Sequence
  Description of Artificial Sequence Synthetic primer
catatggaggcgacccttcccgttttg

SEQ ID NO: 34 - DNA, 32 bp, Artificial Sequence
  Description of Artificial Sequence Synthetic primer
gagctcgcgccagaggaccacccgctccaggg

SEQ ID NO: 35 - DNA, 29 bp, Artificial Sequence
  Description of Artificial Sequence Synthetic primer
ccatggcgaagggcgagtttgttcggacg

SEQ ID NO: 36 - DNA, 32 bp, Artificial Sequence
  Description of Artificial Sequence Synthetic primer
gagctccaggatcttggtgacgacgccggcgc

SEQ ID NO: 37 - DNA, 31 bp, Artificial Sequence
  Description of Artificial Sequence Synthetic primer
catatgagccaaatggaactcatcaagaagc

SEQ ID NO: 38 - DNA, 26 bp, Artificial Sequence
  Description of Artificial Sequence Synthetic primer
ggtacccgcccccagctcaaagcggc

SEQ ID NO: 39 - DNA, 27 bp, Artificial Sequence
  Description of Artificial Sequence Synthetic primer
ccatggatgaacaggctctattagggc

SEQ ID NO: 40 - DNA, 30 bp, Artificial Sequence
  Description of Artificial Sequence Synthetic primer
gagctcgggctttgctctctggaagaacac

SEQ ID NO: 41 - DNA, 58 bp, Artificial Sequence
  Description of Artificial Sequence Synthetic primer
ccatgggcagcagccatcatcatcatcatcacagcagcggcctggtgccgcgcggcag

SEQ ID NO: 42 - DNA, 27 bp, Artificial Sequence
  Description of Artificial Sequence Synthetic primer
ggtaccgagctctagagtggaacctcc

SEQ ID NO: 43 - DNA, 1098 bp, Escherichia coli
atgtttgaaattaatccggtaaataatcgcattcaggacctcacggaacgctccgacgttcttagggggtatcttgactacgacgccaagaaagagcgtctggaagaagtaaacgccgagctggaacagccggatgtctggaacgaacccgaacgcgcacaggcgctgggtaaagagcgttcctccctcgaagccgttgtcgacaccctcgaccaaatgaaacaggggctggaagatgtttctggtctgctggaactggctgtagaagctgacgacgaagaaacctttaacgaagccgttgctgaactcgacgccctggaagaaaaactggcgcagcttgagttccgccgtatgttctctggcgaatatgacagcgccgactgctacctcgatattcaggcggggtctggcggtacggaagcacaggactgggcgagcatgcttgagcgtatgtatctgcgctgggcagaatcgcgtggtttcaaaactgaaatcatcgaagagtcggaaggtgaagtggcgggtattaaatccgtgacgatcaaaatctccggcgattacgcttacggctggctgcgtacagaaaccggcgttcaccgcgtggtgcgtaaaagcccgtttgactccggcggtcgtcgccacacgtcgttcagctccgcgtttgtttatccggaagttgatgatgatattgatatcgaaatcaacccggcggatctgcgcattgacgtttatcgcacgtccggcgcgggcggtcagcacgttaaccgtaccgaatctgcggtgcgtattacccacatcccgaccgggatcgtgacccagtgccagaacgaccgttcccagcacaagaacaaagatcaggccatgaagcagatgaaagcgaagctttatgaactggagatgcagaagaaaaatgccgagaaacaggcgatggaagataacaaatccgacatcggctggggcagccagattcgttcttatgtccttgatgactcccgcattaaagatctgcgcaccggggtagaaacccgcaacacgcaggccgtgctggacggcagcctggatcaatttatcgaagcaagtttgaaagcagggttatga

SEQ ID NO: 44 - protein, 365 aa, Escherichia coli
MFEINPVNNRIQDLTERSDVLRGYLDYDAKKERLEEVNAELEQPDVWNEPERAQALGKERSSLEAVVDTLDQMKQGLEDVSGLLELAVEADDEETFNEAVAELDALEEKLAQLEFRRMFSGEYDSADCYLDIQAGSGGTEAQDWASMLERMYLRWAESRGFKTEIIEESEGEVAGIKSVTIKISGDYAYGWLRTETGVHRLVRKSPFDSGGRRHTSFSSAFVYPEVDDDIDIEINPADLRIDVYRTSGAGGQHVNRTESAVRITHIPTGIVTQCQNDRSQHKNKDQAMKQMKAKLYELEMQKKNAEKQAMEDNKSDIGWGSQIRSYVLDDSRIKDLRTGVETRNTQAVLDGSLDQFIEASLKAGL

SEQ ID NO: 45 - DNA, 426 bp, Escherichia coli
atgggcattaagcgaaagtggtgtggcactattcagtggatttgtccgagtccgtatcggcctcatcatgggcgcaaaaactggtttctgggcattgggcgttttaccgctgatgagcaggaacaatcggatgcaatccgttatgagacgctgcgttcgtcggggccgggcggtcaacatgtcaataaaaccgactcggcggtacgcgccacgcatttggcatccggtattagcgtgaaggttcagtcagagcgtagtcagcatgctaacaagcggctggcacgattgctgattgcctggaagctggagcaacagcaacaggaaaatagcgcggcgctgaaatcgcagcggcgaatgttccatcaccagattgaacgtggcaacccgcgacggacatttacagggatggcttttatcgaaggataa

SEQ ID NO: 46 - protein, 141 aa, Escherichia coli
MGIKRKWCGTIQWICPSPYRPHHGRKNWFLGIGRFTADEQEQSDAIRYETLRSSGPGGQHVNKTDSAVRATHLASGISVKVQSERSQHANKRLARLLIAWKLEQQQQENSAALKSQRRMFHHQIERGNPRRTFTGMAFIEG

SEQ ID NO: 47 - DNA, 615 bp, Escherichia coli
atgatcttgctacaactctcctctgctcaggggccggaagaatgttgtctcgcagtgagaaaagcactggacaggctgattaaagaagctacccgacaggacgtcgcggtaacggtgctggaaacagaaacgggtcgctactctgacacactgcgttcggcgctgatttctctggatggcgataacgcatgggctctaagcgaaagctggtgcggcactattcagtggatttgtccgagtccgtatcggcctcatcatgggcgcaaaaactggtttctgggcattgggcgttttaccgctgatgagcaggaacaatcggatgcaatccgttatgagacgctgcgttcgtcggggccgggcggtcaacatgtcaataaaaccgactcggcggtacgcgccacgcatctggcatccggtattagcgtgaaggttcagtcagagcgcagtcagcatgctaacaaacggctggcgcgattactgattgcctggaagctggaacaacagcaacaggaaaatagcgcggcgctgaaatcgcagcggcgaatgttccatcaccagattgaacgtggcaacccgcgacgaacgtttacagggatggcctttatcgaagggtaa

SEQ ID NO: 48 - protein, 204 aa, Escherichia coli
MILLQLSSAQGPEECCLAVRKALDRLIKEATRQDVAVTVLETETGRYSDTLRSALISLDGDNAWALSESWCGTIQWICPSPYRPHHGRKNWFLGIGRFTADEQEQSDAIRYETLRSSGPGGQHVNKTDSAVRATHLASGISVKVQSERSQHANKRLARLLIAWKLEQQQQENSAALKSQRRMFHHQIERGNPRRTFTGMAFIEG

SEQ ID NO: 49 - DNA, 501 bp, Escherichia coli
gtgctggaaacagaaacgggccgctactctgacacgctgcgttcggcgctgatttctctggatggcgacaacgcatgggcgttaagcgaaagttggtgcggcactattcagtggatttgtctgagtccgtatcggcctcatcatgggcgcaaaaactggtttctgggcattgggcgttttaccgctgatgagcaggaacaatcggatgcaatccgttatgagacgctgcgtccgtcggggccgggcggtcaacatgtcaataaaaccgactcggcggtacgcgccacgcatctggcaaccgggattagcgtgaaggttcagtcagaacgcagccagcatgctaacaaacggctggcgcgattgctgattgcctggaagctggagcagcagcaacaggaaaatagcgcagtgctgaaatcgcagcggcgaatgttccatcaccagattgaacgtggcaacccgcgacgaacgttcacagggatggcttttatcgaaggataa

SEQ ID NO: 50 - protein, 166 aa, Escherichia coli
MLETETGRYSDTLRSALISLDGDNAWALSESWCGTIQWICLSPYRPHHGRKNWFLGIGRFTADEQEQSDAIRYETLRPSGPGGQHVNKTDSAVRATHLATGISVKVQSERSQHANKRLARLLIAWKLEQQQQENSAVLKSQRRMFHHQIERGNPRRTFTGMAFIEG

SEQ ID NO: 51 - protein, 166 aa, Escherichia coli
MLETETGRYSDTLRSALVSLDGDNAWALSESWCGTIQWICPSPYRPHHGRKNWFLGIGRFTADEQEQSDAIRYETLRSSGPGGQHVNKTDSAVRATHLASGISVKVQSERSQHANKRLARLLIAWKLEQQQQENSAALKSQRRMFHHQIERGNPRRTFTGMAFIEG

SEQ ID NO: 52 - DNA, 1314 bp, Oryctolagus cuniculus
atggcggacgaccccagtgctgccgacaggaacgtggaaatctggaagatcaagaagctcattaagagcttggaggcggcccgcggcaatggcaccagcatgatatcattgatcattcctcccaaagaccagatttcccgagtggcaaaaatgttagcagatgaatttggaactgcatccaacattaagtcacgagtaaaccgcctttcagtcctgggagccattacatctgtacaacaaagactcaaactttataacaaagtacctccaaatggtctggttgtttactgtggaacaattgtaacagaagaaggaaaggaaaagaaagtcaacattgactttgaacctttcaaaccaattaatacgtcattgtatttgtgtgacaacaaattccatacagaggctcttacagcactactttcagatgatagcaagtttggcttcattgtaatagatggtagtggtgcactttttggcacactgcagggaaatacaagagaagtcctgcacaaattcactgtggatctcccaaagaaacacggtagaggaggtcagtcagccttgcgttttgcccgtttaagaatggaaaagcgacacaactatgttcggaaagtagcagagactgctgtacagctgtttatttctggggacaaagtgaatgtggctggtctcgttttagctggatcagctgactttaaaactgaactaagtcaatctgatatgtttgaccagaggttgcaatcaaaagttttaaaattagttgatatatcctatggcggtgaaaatggattcaaccaagctattgagttatctactgaggtcctctccaacgtgaaattcattcaagagaagaaattaataggacgatactttgatgaaatcagtcaagacacgggcaagtactgttttggagttgaagatacgctaaaagctttggaaatgggagccgtagaaattctaatagtctatgaaaatttggatataatgagatacgttcttcattgccaaggcacagaagaggagaaaattctttacctaactccagaacaagagaaggataaatctcatttcacagacaaagagacaggacaggaacatgagctgattgagagcatgcccctgttggaatggtttgctaacaactataaaaaatttggagctacattggaaattgtcacagataagtcacaagaaggatcccagtttgtgaaaggatttggtggaattggaggtatcttgcggtaccgagtagatttccagggaatggaatatcaaggaggagacgatgaattttttgaccttgatgactactag

SEQ ID NO: 53 - protein, 437 aa, Oryctolagus cuniculus
MADDPSAADRNVEIWKIKKLIKSLEAARGNGTSMISLIIPPKDQISRVAKMLADEFGTASNIKSRVNRLSVLGAITSVQQRLKLYNKVPPNGLVVYCGTIVTEEGKEKKVNIDFEPFKPINTSLYLCDNKFHTEALTALLSDDSKFGFIVIDGSGALFGTLQGNTREVLHKFTVDLPKKHGRGGQSALRFARLRMEKRHNYVRKVAETAVQLFISGDKVNVAGLVLAGSADFKTELSQSDMFDQRLQSKVLKLVDISYGGENGFNQAIELSTEVLSNVKFIQEKKLIGRYFDEISQDTGKYCFGVEDTLKALEMGAVEILIVYENLDIMRYVLHCQGTEEEKILYLTPEQEKDKSHFTDKETGQEHELIESMPLLEWFANNYKKFGATLEIVTDKSQEGSQFVKGFGGIGGILRYRVDFQGMEYQGGDDEFFDLDDY

SEQ ID NO: 54 - DNA, 1767 bp, Oryctolagus cuniculus
ctggcggcggcggccgaggcccagcgtgaccacctcagcgcggccttcagccggcagctcaacgtcaacgccaaacctttcgtgcccaacgtccacgccgcggagttcgtaccgtctttcctgcggggcccggccccgcctccagccccggctggcgccgccggcaacaaccacggagcgggcagcgtcgcgggaggcccttcggcacctgtggaatcctctcaagaggaacagtcattgtgtgaaggctccatttcagctgttagcatggaactttcagaacctgttgtagagaacggagagacagaaatgtccccagaagaatcatgggagcacaaagaagaaataagtgaggcagagccagggggtggctccctgggagatggaaggccaccggaggaaggtgcccaagaaatgatggaggaggaagaggaaatgccaaagcccaaatctgtagctgcgcctcctggtgcccctaaaaaagaacatgtaaatgtagtgtttattgggcatgtagatgctggcaagtcaaccattggaggccaaataatgtatttgactggaatggttgataaaaggacacttgagaaatatgaaagagaagctaaagaaaaaaacagagaaacttggtacttgtcttgggccctagatacaaatcaggaagaacgagacaaaggtaaaacagtcgaagtgggtcgtgcctactttgaaacagaaaagaagcatttcacaattctagacgcccctggccacaagagttttgtcccaaatatgattggtggcgcctctcaagctgatttggctgtgctggtcatctctgccaggaaaggagagtttgaaactggatttgaaaaaggaggacagacaagagaacacgcaatgttggcaaagacagcaggtgtaaagcacttaattgtgcttattaataagatggatgacccaacagtgaattggagcaacgagagatatgaagaatgtaaagagaaactagtgccatttttgaaaaaagttggcttcaatcccaaaaaggacattcactttatgccctgctcaggactgactggagcaaatctcaaagagcaatcagatttctgtccttggtacattggattaccatttattccatatctggataatttgccaaacttcaatagatcagttgatggaccaatcagactgccgattgtggataagtacaaggatatgggcactgtggtcctgggaaagctggaatcgggatctatttgtaaaggccagcagctagtgatgatgccgaacaagcacaacgtggaagttcttggaatactttctgatgatgtagaaactgattctgtagccccaggtgagaacctgaaaatcagactcaaaggaattgaggaagaagagattcttccaggattcatcctttgtgatcttaataatctttgccattctggacgcacatttgatgcccagatagtgattatagagcacaaatccatcatctgcccagggtacaatgcggtgctgcatattcatacctgtattgaggaagtcgagataacagccttaatctgcttggtagacaaaaagtcaggagagaaaagcaagactcggccccgttttgtgaaacaagatcaagtgtgcattgcccgtcttcggacagcaggaaccatctgccttgagacctttaaggacttccctcagatgggtcgttttaccttaagagatgagggtaagaccattgcaattggaaaagttctgaaactggttccagaaaaagactaa

SEQ ID NO: 55 - protein, 588 aa, Oryctolagus cuniculus
LAAAAEAQRDHLSAAFSRQLNVNAKPFVPNVHAAEFVPSFLRGPAPPPAPAGAAGNNHGAGSVAGGPSAPVESSQEEQSLCEGSISAVSMELSEPVVENGETEMSPEESWEHKEEISEAEPGGGSLGDGRPPEEGAQEMMEEEEEMPKPKSVAAPPGAPKKEHVNVVFIGHVDAGKSTIGGQIMYLTGMVDKRTLEKYEREAKEKNRETWYLSWALDTNQEERDKGKTVEVGRAYFETEKKHFTILDAPGHKSFVPNMIGGASQADLAVLVISARKGEFETGFEKGGQTREHAMLAKTAGVKHLIVLINKMDDPTVNWSNERYEECKEKLVPFLKKVGFNPKKDIHFMPCSGLTGANLKEQSDFCPWYIGLPFIPYLDNLPNFNRSVDGPIRLPIVDKYKDMGTVVLGKLESGSICKGQQLVMMPNKHNVEVLGILSDDVETDSVAPGENLKIRLKGIEEEEILPGFILCDLNNLCHSGRTFDAQIVIIEHKSIICPGYNAVLHIHTCIEEVEITALICLVDKKSGEKSKTRPRFVKQDQVCIARLRTAGTICLETFKDFPQMGRFTLRDEGKTIAIGKVLKLVPEKD

SEQ ID NO: 56 - DNA, 1317 bp, Drosophila melanogaster
atgtctggcgaggaaacgtctgccgatcgcaatgtcgagatctggaaaatcaagaagctcatcaagagcctggaaatggcccgcggcaatggaaccagcatgatttctttgattattccgccaaaggatcaaatctcgcgcgtcagcaagatgttggccgatgagtttggaacggcgtcgaacatcaagtcgcgtgtaaatcggttgtccgtcctcggtgccattacgtcggtacagcacagactcaaattatacaccaaagtgcctcccaacggtttggtcatctactgcggcacaatagtcacagaggagggcaaggagaagaaggtgaacatagactttgagccattcaagcccataaacacgtcgctctacctctgcgacaacaagttccacacggaggccctcactgccctgctcgccgacgacaacaaatttggattcatcgtgatggatggtaacggagcgctattcggtacccttcagggcaacacgcgcgaggtgctccacaagttcaccgtcgatctgccgaagaagcacggtcgtggtggtcagtccgcccttcgtttcgcccgtctgcgtatggagaagcgccacaactacgtgcggaaggtcgccgaggtggccacccagctcttcatcacgaacgacaagcccaacattgccggactcatcctggctggtagtgcggatttcaagactgagcttagtcagtctgatatgttcgatcctcgtttgcaatcaaaagtcatcaagctggtggacgtgtcgtatggtggggaaaacggttttaaccaggccattgaactggcggccgaatcattgcagaacgttaaattcatacaggagaagaaactcattggtcgctactttgatgaaatttctcaggatactggcaaatactgttttggagtggaggacactttgcgggcactggaacttggctctgtagagactctcatttgttgggagaacctggatattcaacgttatgttctcaagaatcatgccaactcgacgtcaacgacagtattacatttgacgcccgagcaggaaaaggacaagtcgcacttcactgacaaggagagcggggtagaaatggagctgattgagtctcagccgctgctggaatggctggcaaacaactacaaaatgttcggcgccacactggagattatcacggataagtcccaggaaggaagtcagttcgtgcgaggtttcggtggaatcggcggtatcttacgctacaaggtggatttccagagtatgcagctcgatgaattggacaatgatggcttcgatctagatgattactag

SEQ ID NO: 57 - protein, 438 aa, Drosophila melanogaster
MSGEETSADRNVEIWKIKKLIKSLEMARGNGTSMISLIIPPKDQISRVSKMLADEFGTASNIKSRVNRLSVLGAITSVQHRLKLYTKVPPNGLVIYCGTIVTEEGKEKKVNIDFEPFKPINTSLYLCDNKFHTEALTALLADDNKFGFIVMDGNGALFGTLQGNTREVLHKFTVDLPKKHGRGGQSALRFARLRMEKRHNYVRKVAEVATQLFITNDKPNIAGLILAGSADFKTELSQSDMFDPRLQSKVIKLVDVSYGGENGFNQAIELAAESLQNVKFIQEKKLIGRYFDEISQDTGKYCFGVEDTLRALELGSVETLICWENLDIQRYVLKNHANSTSTTVLHLTPEQEKDKSHFTDKESGVEMELIESQPLLEWLANNYKMFGATLEIITDKSQEGSQFVRGFGGIGGILRYKVDFQSMQLDELDNDGFDLDDY

SEQ ID NO: 58 - DNA, 1314 bp, Saccharomyces cerevisiae
atggataacgaggttgaaaaaaatattgagatctggaaggtcaagaagttggtccaatctttagaaaaagctagaggtaatggtacttctatgatttccttagttattcctcctaagggtctaattccactgtaccaaaaaatgttaacagatgaatatggtactgcctcgaatattaaatctagggttaatcgtctttccgttttatctgctatcacttccacccaacaaaagttgaagctatataatactttgcccaagaacggtttagttttatattgtggtgatatcatcactgaagatggtaaagaaaaaaaggtcacttttgacatcgaaccttacaaacctatcaacacatccttatatttgtgtgataacaaatttcatacagaagttctttcggaattgcttcaagctgacgacaagttcggttttatagtcatggacggtcaaggtactttgtttggttctgtgtccggtaatacgagaactgttttacataaatttactgtcgatctgccaaaaaagcatggtagaggtggtcaatctgcgcttcgttttgctcgtttaagagaagaaaaaagacataattatgtgagaaaggtcgccgaagttgctgttcaaaattttattactaatgacaaagtcaatgttaagggtttaattttagctggttctgctgactttaagaccgatttggctaaatctgaattattcgatccaagactagcatgtaaggttatttccatcgtggatgtttcttatggtggtgaaaacggtttcaaccaggctatcgaactttctgccgaagcgttggccaatgtcaagtatgttcaagaaaagaaattattggaggcatattttgacgaaatttcccaggacactggtaaattctgttatggtatagatgatactttaaaggcattggatttaggtgcagtcgaaaaattaattgttttcgaaaatttggaaactatcagatatacatttaaagatgccgaggataatgaggttataaaattcgctgaaccagaagccaaggacaagtcgtttgctattgacaaagctaccggccaagaaatggacgttgtctccgaagaacctttaattgaatggctagcagctaactacaaaaacttcggtgctaccttggaattcatcacagacaaatcttcagaaggtgcccaatttgtcacaggttttggtggtattggtgccatgctgcgttacaaagttaattttgaacaactagttgatgaatctgaggatgaatattatgacgaagatgaaggatccgactatgatttcatttaa

SEQ ID NO: 59 - protein, 437 aa, Saccharomyces cerevisiae
MDNEVEKNIEIWKVKKLVQSLEKARGNGTSMISLVIPPKGLIPLYQKMLTDEYGTASNIKSRVNRLSVLSAITSTQQKLKLYNTLPKNGLVLYCGDIITEDGKEKKVTFDIEPYKPINTSLYLCDNKFHTEVLSELLQADDKFGFIVMDGQGTLFGSVSGNTRTVLHKFTVDLPKKHGRGGQSALRFARLREEKRHNYVRKVAEVAVQNFITNDKVNVKGLILAGSADFKTDLAKSELFDPRLACKVISIVDVSYGGENGFNQAIELSAEALANVKYVQEKKLLEAYFDEISQDTGKFCYGIDDTLKALDLGAVEKLIVFENLETIRYTFKDAEDNEVIKFAEPEAKDKSFAIDKATGQEMDVVSEEPLIEWLAANYKNFGATLEFITDKSSEGAQFVTGFGGIGAMLRYKVNFEQLVDESEDEYYDEDEGSDYDFI

SEQ ID NO: 60 - DNA, 1137 bp, Thermus thermophilus
ttgcgcctcgcttcgcaatctgctatcctggtaaaggtatggacctggaacgcctcgcgcaacgcctggaaggcctcagggggtatctttgacatcccccaaaaggaaacccgtctaaaagagctggagcggcgcctcgaggacccctccctctggaacgatcccgaggccgcccgcaaggtgagccaggaggccgcccgcctccggcgcaccgtggacaccttccgctccctggaaagcgacctccagggccttttggagctcatggaggagcttcccgccgaggaacgggaggccctcaagcccgagctggaggaggccgcgaagaagctggacgagctctaccaccagaccctcctcaacttcccccacgcggagaagaacgccatcctcaccatccagcccggggccgggggcacggaggcctgcgactgggcggagatgctcctaaggatgtacacccgcttcgccgagcgccagggcttccaggtggaggtggtggacctcacccctgggcccgaggcgggcattgactacgcccagatcctggtcaagggggagaacgcctacggcctcctttcccccgaggccggggtgcaccgcctggtgcgcccttccccctttgacgcctcgggccgccgccacacctccttcgccggggtggaggtgatccccgaggtggacgaggaggtggaggtggtgctcaagcccgaggagctccgcattgacgtgatgcgggcctcggggcccgggggccagggggtgaacaccacggactcggcggtgcgggtggtccacctgcccacggggatcaccgtgacctgccagaccacgcggagccagatcaagaacaaggaactcgccctcaagatcctcaaggcccgcctctacgagctggagcggaagaagcgggaggaagagctcaaggccctgaggggcgaggtgcggcccatagagtggggaagccagatccggagctacgtcctggacaagaactacgtcaaggaccaccgcaccgggctcatgcgccacgacccggaaaacgtcctggacggggacctcatggacctgatctgggcgggcctggagtggaaggcgggccgccgccaggggacggaggaggtggaggcggagtag

SEQ ID NO: 61 - protein, 378 aa, Thermus thermophilus
MRLASQSAILVKVWTWNASRNAWKASGGIFDIPQKETRLKELERRLEDPSLWNDPEAARKVSQEAARLRRTVDTFRSLESDLQGLLELMEELPAEEREALKPELEEAAKKLDELYHQTLLNFPHAEKNAILTIQPGAGGTEACDWAEMLLRMYTRFAERQGFQVEVVDLTPGPEAGIDYAQILVKGENAYGLLSPEAGVHRLVRPSPFDASGRRHTSFAGVEVIPEVDEEVEVVLKPEELRIDVMRASGPGGQGVNTTDSAVRVVHLPTGITVTCQTTRSQIKNKELALKILKARLYELERKKREEELKALRGEVRPIEWGSQIRSYVLDKNYVKDHRTGLMRHDPENVLDGDLMDLIWAGLEWKAGRRQGTEEVEAE

SEQ ID NO: 62 - protein, 310 aa, Alicyclobacillus acidocaldarius
MPLDPVIQQVLDQLNRMPAPDYKHLSAQQFRSQQSLFPPVKKEPVAEVREFDMDLPGRTLKVRMYRPEGVEPPYPALVYYHGGGWVVGDLETHDPVCRVLAKDGRAVVFSVDYRLAPEHKFPAAVEDAYDALQWIAERAADFHLDPARIAVGGDSAGGNLAAVTSILAKERGGPALAFQLLIYPSTGYDPAHPPASIEENAEGYLLTGGMMLWFRDQYLNSLEELTHPWFSPVLYPDLSGLPPAYIATAQYDPLRDVGKLYAEALNKAGVKVEIENFEDLIHGFAQFYSLSPGATKALVRIAEKLRDALA

SEQ ID NO: 63 - protein, 4 aa, Artificial Sequence
  Description of Artificial Sequence Synthetic peptide
HGGG

SEQ ID NO: 64 - DNA, 24 bp, Artificial Sequence
  Description of Artificial Sequence Synthetic oligonucleotide
tcctgtgtgaaattgttatccgct

SEQ ID NO: 65 - protein, 5 aa, Artificial Sequence
  Description of Artificial Sequence Synthetic peptide
DDDDK

(19) United States
(12) Patent Application Publication
(10) Pub. No.: US 2009/0258348 A1
(43) Pub. Date: Oct. 15, 2009

(54) ESTERASES FOR MONITORING PROTEIN BIOSYNTHESIS IN VITRO

(75) Inventors: Mathias Sprinzl, Bayreuth (DE); Dmitry Agafonov, Göttingen (DE); Kersten Rabe, Kirchhatten (DE)

Correspondence Address: FOLEY AND LARDNER LLP;SUITE 500, 3000 K STREET NW, WASHINGTON, DC, 20007, US

(73) Assignee: UNIVERSITÄT BAYREUTH, Bayreuth (DE)

(21) Appl. No.: 11/815,416

(22) PCT Filed: Feb. 2, 2006
(86) PCT No.: PCT/EP06/00927

§ 371 Date: Aug. 2, 2007

(30) Foreign Application Priority Data
Feb. 2, 2005 (EP) 05002200.3

(51) Int. Cl. C12Q 1/68; C12N 15/74; C07K 2/00; G01N 33/53

(52) U.S. Cl. 435/6; 435/320.1; 530/300; 530/350; 435/7.9

(57) ABSTRACT

The present invention relates to the use of an esterase for monitoring and/or tracking the synthesis of a protein, polypeptide or peptide in a cell-free translation system or in an in vivo expression system in which the synthesis of a protein, polypeptide or peptide can occur, wherein said monitoring and/or tracking comprises the detection of the function of said esterase. The present invention further relates to a vector comprising a nucleic acid molecule coding for an esterase and expressing an esterase fusionprotein. Moreover, the present invention relates to a vector comprising a nucleic acid molecule coding for an esterase and comprising in frame at least one multiple cloning site for a further protein/polypeptide/peptide, to be expressed in form of a fusion protein comprising said esterase (esterase activity) and said further proteinaceous peptide structure. The present invention also provides for a protein, polypeptide or peptide encoded by the vectors of the present invention. Additionally, the present invention relates to a kit comprising a vector of the present invention or a nucleic acid molecule as comprised by the vectors of the present invention. Also disclosed is a method for monitoring and/or tracking the synthesis of a protein, polypeptide or peptide in a cell-free translation system or in an in vivo expression system, comprising the step of detecting the function of an esterase. The present invention also teaches a method for immobilising a protein, polypeptide or peptide comprising the steps of (a) tagging said protein, polypeptide or peptide with an esterase and (b) binding said esterase to an esterase inhibitor, wherein said esterase inhibitor is immobilized on a solid substrate. Moreover, the present invention relates to uses of the vectors of the present invention or the nucleic acid molecules comprised therein for the preparation of a kit or for monitoring and/or tracking the synthesis of a protein, polypeptide or peptide in a cell-free translation system, whereby the monitoring and/or tracking comprises the detection of the function of said esterase.

The present invention relates to the use of an esterase for monitoring and/or tracking the synthesis of a protein, polypeptide or peptide in a cell-free translation system or in an in vivo expression system in which the synthesis of a protein, polypeptide or peptide can occur, wherein said monitoring and/or tracking comprises the detection of the function or activity of said esterase; preferably said function or activity is the enzymatic function of said esterase. The present invention further relates to a vector comprising a nucleic acid molecule coding for an esterase and expressing an esterase fusion protein. Moreover, the present invention relates to a vector comprising a nucleic acid molecule coding for an esterase and comprising in frame at least one multiple cloning site for a further protein/polypeptide/peptide, to be expressed in form of a fusion protein comprising said esterase (esterase activity) and said further proteinaceous peptide structure. The present invention also provides for a protein, polypeptide or peptide encoded by the vectors of the present invention. Additionally, the present invention relates to a kit comprising a vector of the present invention or a nucleic acid molecule as comprised by the vectors of the present invention. Also disclosed is a method for monitoring and/or tracking the synthesis of a protein, polypeptide or peptide in a cell-free translation system or in an in vivo expression system, comprising the step of detecting the function of an esterase. The present invention also teaches a method for immobilising a protein, polypeptide or peptide comprising the steps of (a) tagging said protein, polypeptide or peptide with an esterase and (b) binding said esterase to an esterase inhibitor, wherein said esterase inhibitor is immobilized on a solid substrate. Moreover, the present invention relates to uses of the vectors of the present invention or the nucleic acid molecules comprised therein for the preparation of a kit or for monitoring and/or tracking the synthesis of a protein, polypeptide or peptide in a cell-free translation system, whereby the monitoring and/or tracking comprises the detection of the function of said esterase.

Cell-free (coupled transcription/)translation systems for the in vitro synthesis of proteins are used either for the production of (functionally active) proteins or for studying of protein biosynthesis (in vitro) (Spirin (2002), Cell-Free Translation Systems. Springer Verlag, Berlin.). Normally, the cell-free (coupled transcription/)translation systems are derived from prokaryotic cells such as E. coli cells (e.g. Zubay (1973), Imm. Rev. Genet. Vol. 7, page 267) or from eukaryotic cells such as rabbit reticulocytes (e.g. Pelham (1976), Eur. J. Biochem. Vol. 131, page 289) and wheat germ cells (e.g. Spirin (1990), American Society for Microbiology, 56-70; Stiege (1995), J. Biotechnol. 41:81-90). The use of this systems became a standard technology in laboratory praxis (e.g., Baranov (1989), Gene, 84, 463-436; Endo (1992), J. Biotechnol., 25, 221-230) and the proteins produced by this systems are widely employed in biochemical, biostructural and pharmaceutical uses, not only in fundamental research but also in the biochemical, chemical and pharmaceutical industry.

To achieve high yields of the desired proteins (to be expressed) and in order to facilitate their isolation, optimization of the employed (coupled transcription/)translation systems is required. In particular, in often used (coupled transcription/)translation systems the monitoring of the synthesis of the proteins is necessary.

Accordingly, there is a need for useful reporter proteins/molecules/groups and protein tags. Up to now, several representatives of this reporters have been tried, however, with limitations and down sides.

One example of a marker/monitor reporter to detect protein expression, in particular in bacterial or eukaryotic in vitro translation systems/cell-free translation systems, is the green fluorescent protein (GFP) (Kolb (1996), Biotechnology Letters, 18, 1447-1452.). Others comprise, firefly luciferase (Kolb (1994), EMBO J., 13, 3631-3637.), dihydrofolate reductase (DHFR) (Endo (1992), J. Biotechnol., 25, 221-230; Kudlicki (1992), Biochem., 206, 389-393.), chloramphenicol acetyl transferase (CAT) (Kigawa (1991), J. Biochem. (Tokyo), 110, 166-168) and β-galactosidase (NotI (1980), J. Bacteriol., 144, 291-299.). Several modifications of these markers, which are often calorimetrically and/or fluorimetrically assessed, are employed in the art.

Especially green fluorescent protein (GFP), in particular in form of enhanced green fluorescent protein (eGFP), is one of the most commonly used reporter group. This protein requires time (several hours) for maturation (Coxon (1995), Chem. Biol., 2, 119-121.) and the fluorophor is formed post-translationally by oxidation with molecular oxygen. Therefore, the direct in-situ monitoring of protein expression (in cell-free systems, like (coupled transcription/)translation systems) is not possible. Moreover, the sensitivity of the detection of (e)GFP-labelled proteins is quite low and has also quantitative limits: About 100 mg/ml can be visualized directly with the naked eye, 0.1 mg/ml can be detected by fluorometer, and 1 μg of GFP is visible as a band in electrophoresis gel (Chekulayeva (2001), Biochem Biophys Res Commun., 280, 914-917).

Similarly, even though the activity of firefly luciferase can be measured directly in cell-free translation systems (Kolb (2000), J. Bio.l Chem., 275, 16597-16601) it dramatically looses enzymatic activity at temperatures above 30° C. However, often higher temperatures are required (e.g. 37° C. in E. coli cell free extracts (see also in the appended examples)) and therefore, temperature sensitivity of reporter proteins is of great disadvantage.

For the detection of chloramphenicol acetyl transferase activity, a radioactive substrate and expensive equipments are required (Young (1985), DNA, 4, 469-475) and therefore said detection is complicated.

Further to other difficulties in the usage of the above listed reporters, their main disadvantage (for their practical use) is their lack of thermostability as they are derived from organisms living at normal temperature ranges (10-40° C.; mesophilic organisms). Along with temperature limitation, direct monitoring of the other above listed enzymatic activities in complex (coupled transcription/)translation mixtures is not possible.

Thus, the technical problem underlying the present invention is the provision of reliable means and methods for the assessment of the capabilities of a given cell-free translation system and/or cell free transcription/translation systems in vitro protein/peptide synthesis.

The solution to the above technical problem is achieved by providing the embodiments characterized in the claims.

Accordingly, the present invention relates to the use of an esterase for monitoring and/or tracking the synthesis of a protein, polypeptide or peptide in a cell-free translation system or in an in vivo expression system in which the synthesis of a protein, polypeptide or peptide can occur, wherein said monitoring and/or tracking comprises the detection of the function of said esterase.

The present invention solves the above identified technical problem since, as documented herein below and in the appended examples, it was surprisingly found that esterases (or enzymatic esterase activities of polypeptides or functional fragments of esterases comprising esterase activity) may be employed as markers for the determination of the efficacy and/or function of cell-free translation systems or in in vivo expression systems. Accordingly, and in particular embodiments, the present invention allows for the expression of a fusion construct comprising a desired moiety “X” and an esterase moiety. In context of the present invention, the term “esterase moiety” refers to a full length esterase, as well as to a fragment thereof displaying esterase activity/esterase function.

It was found that the synthesis of a protein, polypeptide or peptide in a cell-free translation system in an in vivo expression system can be easily and efficiently be detected when said protein, polypeptide or peptide is synthesized with, preferably, a covalently attached/bound esterase (or an esterase activity). Accordingly, the present invention provides, in one embodiment, for fusionproteins/fusionpolypeptides to be expressed in cell-free translation systems, whereby said fusionproteins/fusionpolypeptides comprise an esterase activity as one part of said fusionproteins/fusionpolypeptides and the protein/polypeptide/peptide to be expressed or desired to be expressed in the translation system as at least one further part. As will be detailed below, the fusionproteins/fusionpolypeptides are, accordingly, expressed in said translation system in the format “esterase-X” or “X-esterase”, whereby “esterase” denotes the esterase (or esterase-activity) as defined herein and “X” denotes the a protein, polypeptide or peptide desired to be expressed in the translation system. Accordingly, the desired protein, polypeptide or peptide (to be expressed from a desired “target gene”) may be covalently bound to the N- or C-terminus of the herein defined esterase (or a functional fragment of said esterase, displaying esterase activity/esterase function) In the appended, not limiting example, as esterase/esterase activity esterase 2 of A. acidocaldarius (Est2) is employed and “X” is exemplified by green fluorescent protein (GFP). The person skilled in the art is readily in the position to replace said GFP by any desired protein/polypeptide or peptide “X” without deviating from the gist of the present invention.

The terms “esterase-X” and “X-esterase” are not limited to fusion proteins which comprise merely the esterase (or esterase activity) and the protein, polypeptide or peptide to be expressed. Said terms also comprise, inter alia, the possibility that also “linker” structures are comprised in said fusionproteins/fusionpolypeptides. Also comprised in context of this invention is the possibility that the esterase (or esterase activity) be expressed in context of fusionpolypeptides whereby not only one protein, polypeptide or peptide is covalently expressed with said esterase. Therefore, the invention also provides for the use of an esterase for monitoring/tracking the syntheses of multiple proteins or polypeptides or peptides. Accordingly, also polypeptide structures, in form of fusionproteins/fusionpolypeptides, may be expressed in the cell-free translation system in the format “esterase-X-X′ ”, “X-X′-esterase” or “X′-esterase-X”. In this respect, “X” denotes one particular protein/polypeptide/peptide to expressed and “X′” denotes a further protein/polypeptide/peptide. Also, the esterase (or esterase activity), X and X′ may be separated by “linkers/linker structures”, preferably by cleavable linker structures. As will be detailed below, such linkers/linker structures are known in the art and consist preferably of chemically and/or enzymatically cleavable structures. In context of this invention, it is of note that “X” does not only relate to full-length proteins desired to be synthesized in the cell-free translation systems but may also denote fragments of full-length proteins/polypeptides, preferably said fragments are “functional fragments”, i.e. fragments comprising, when expressed a certain activity. Said activity may be, but is not limited to, an enzymatic activity of said fragment. Yet, the present invention is also useful in the monitoring/tracking of the in vitro synthesis of “peptides”. Such peptides may comprise a minimal amount of amino acid residues, but comprise, preferably at least 10 amino acid residues, more preferably at least 12 amino acid residues, more preferably at least 15 amino acid residues, more preferably at least 20 amino acid residues, more preferably at least 30 amino acid residues, more preferably at least 40 amino acid residues and most preferably at least 50 amino acid residues. Such peptides to be expressed may, inter alia, be useful in immunization approaches.

In context of the present invention, the meaning of the term “protein(s)” may also include “peptide(s)” or “polypeptide(s)”. The meaning of the terms “protein(s)”, “peptide(s)” or “polypeptide(s)” are well known in the art (see,e.g., Stryer (1995), Biochemistry, 4^{th} edition). As known in the art, the term “peptide” comprises joined amino acid residues, whereby the alpha-carboxyl group of one amino acid is joined to the alpha-group of another amino acid by a peptide bond (amide bond); see also Stryer ((1995), loc. cit.). In accordance with the invention, the term “peptide(s)” comprises any such joined amino acid residues, whereby at least three, preferably at least five, most preferably at least seven amino acids (amino acid residues) are linked via said peptide bond (amide bond). The term polypeptide comprises, in accordance with this invention, at least 15 joined amino acid residues, more preferably at least 20 amino acid residues. Accordingly, joined amino acid residues comprising 3 to 14 amino acid residues are to be considered in accordance with this invention as “peptide” whereas joined amino acid residues comprising 15 or more amino acid residues are considered as polypeptides. The term “protein” is used as synonym with the term “polypeptide”, whereas the term “protein” also may comprise a specific biological, biochemical or pharmaceutical function exerted by said protein. However, the person skilled in the art is aware that a protein is a polypeptide. The terms “protein”, “peptide” and “polypeptide” also comprise molecules comprising at least one unnaturally occurring amino acid residue or at least one unusual amino acid residue and is not limited to proteinaceous structures comprising the twenty normally occurring amino acid residues; see also Stryer ((1995), loc. cit.).

The proteins, polypeptides and peptides as mentioned herein are the desired gene products to be produced by the target genes employed in the in vitro translation or in vivo expression systems as discussed and/or described herein. The term “target gene”, accordingly, means a gene to be expressed, in particular in the in vitro systems, preferably in the in vitro translation systems or the in vivo expression systems as discussed and described herein and as also known in the art. An exemplified in vivo expression system may be, e.g. a system based on prokaryotic hosts, like E. coli.

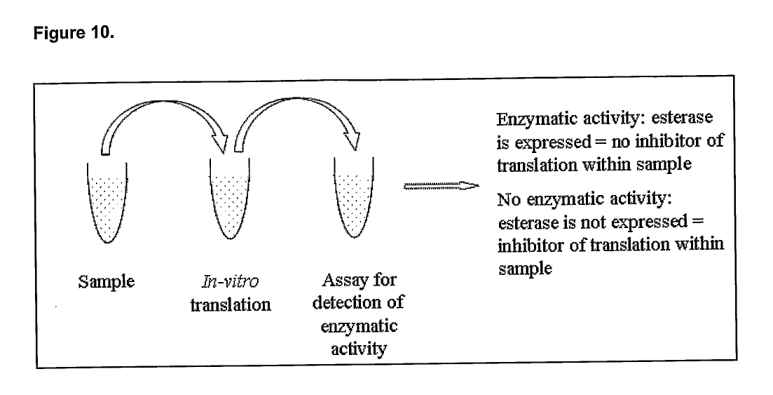

In context of the present invention, it was also found that the synthesis of a protein, polypeptide or peptide in a cell-free translation system can be easily and efficiently be detected when said protein, polypeptide or peptide to be synthesized is an esterase (or an esterase activity) or when said protein structure/polypeptide/peptide to be synthesized is covalently linked to said esterase/esterase activity. Therefore, in context of the present invention, it is envisaged that the esterase (or esterase activity) is either solely expressed in the employed cell-free translation system or that said esterase (esterase activity) is expressed in form of a fusion construct as described herein. In case the esterase is solely expressed, the use of an esterase for monitoring and/or tracking the synthesis of a protein, polypeptide or peptide in a cell-free translation system may be employed for determining the efficacy of said cell-free translation system per se. Accordingly, in one embodiment, the present invention provides for a method for screening substances that influence the function of protein biosynthesis in cell-free (coupled transcription/) translation systems. Said influence may be either the inhibiting or enhancing of the transcription step of in vitro biosynthesis of proteins and/or the inhibiting or enhancing the translation step of in vitro biosynthesis of proteins. An example of a method for screening the inhibitory effect of a substance on the translation step within a cell-free (coupled transcription/) translation system is shown in FIG. 10.

The appended examples document that the present invention provides for a unique monitoring/tracking system for the expression of proteins, polypeptides or peptides in in vitro translation system. Furthermore, also a unique monitoring/tracking system for the expression of proteins, polypeptides or peptides in cellular expression systems is provided by the present invention. However, the present invention is particularly useful for the monitoring and/or tracking of the expression of proteins, polypeptides or peptides in in vitro translation systems/cell free translation systems. Cellular expression systems to be employed with respect to the present invention are known in the art and comprise, inter alia, bacterial cells (e.g. cells form E. coli) or eukaryotic cells (e.g. Yeast cells, CHO cells, HELA cells). A person skilled in the art is immediately able to replace in vitro translation systems as employed herein above by said cellular expression systems known in the art. The synthesis of the desired protein, polypeptide or peptide may be performed by heterologous or homologous expression in said systems. Again, the esterase/esterase activity as provided herein is used in this context as marker system for the monitoring and/or tracking of protein-bio synthesis or peptide-biosynthesis.

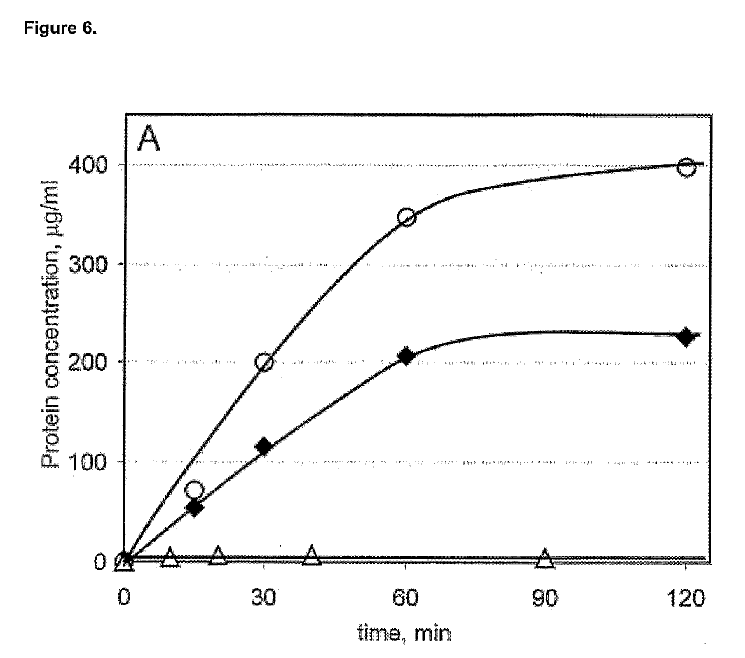

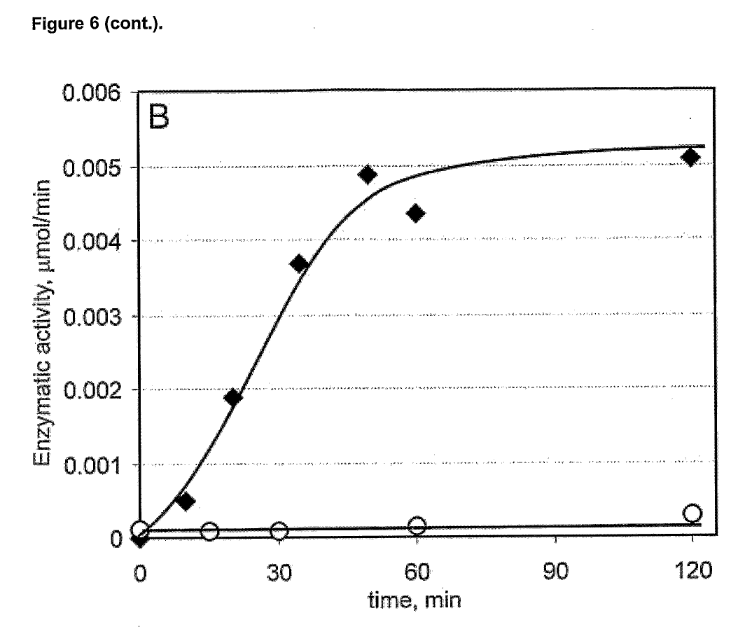

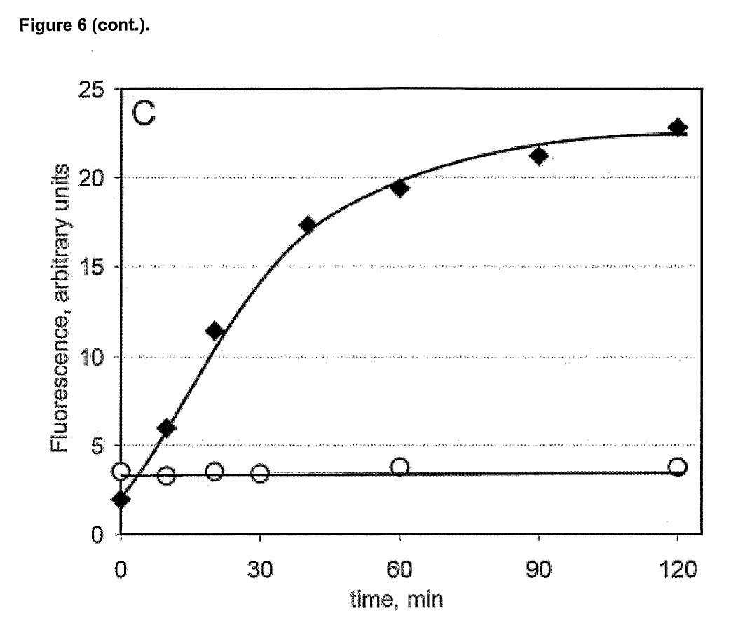

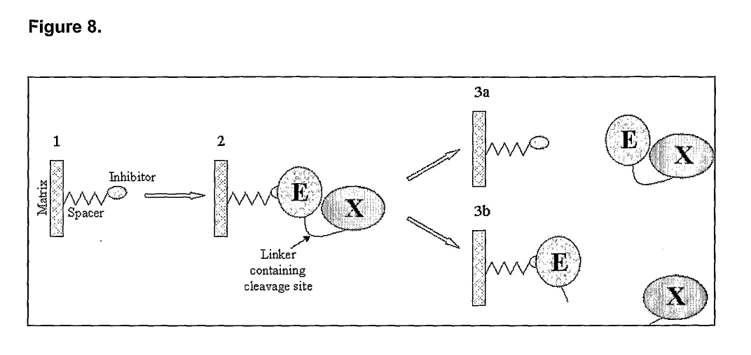

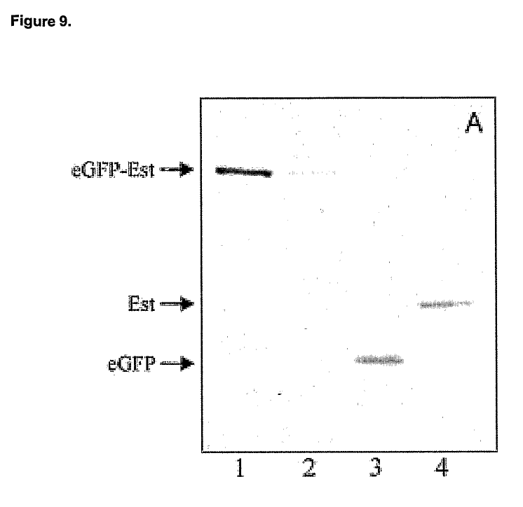

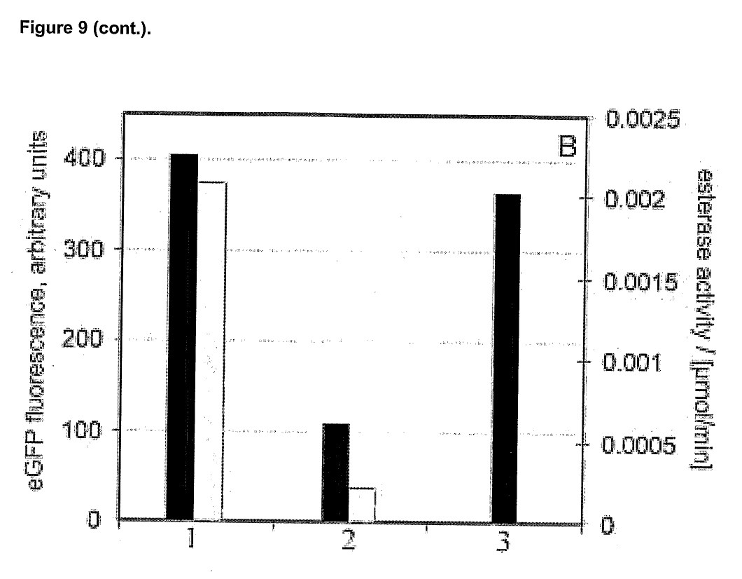

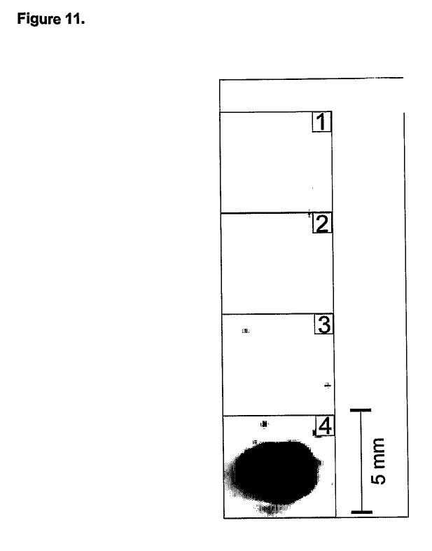

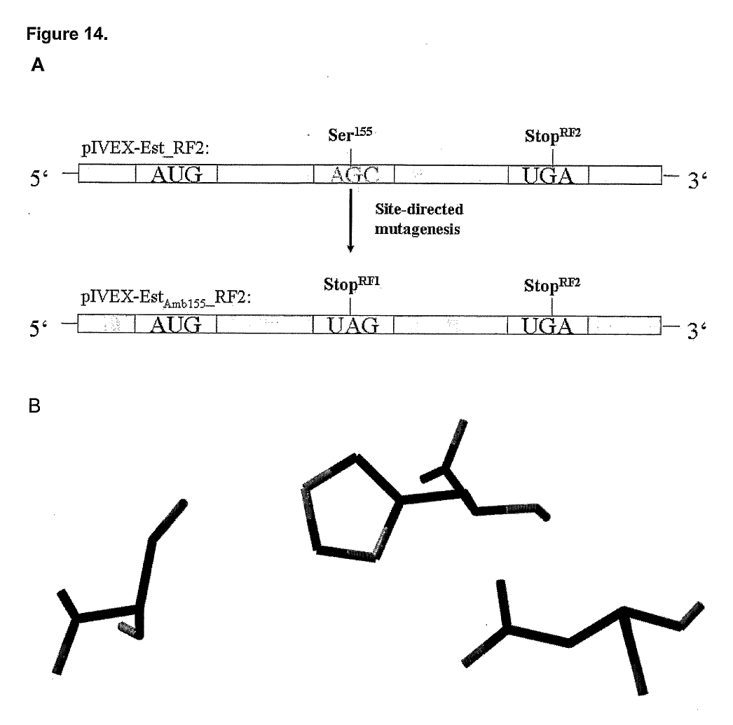

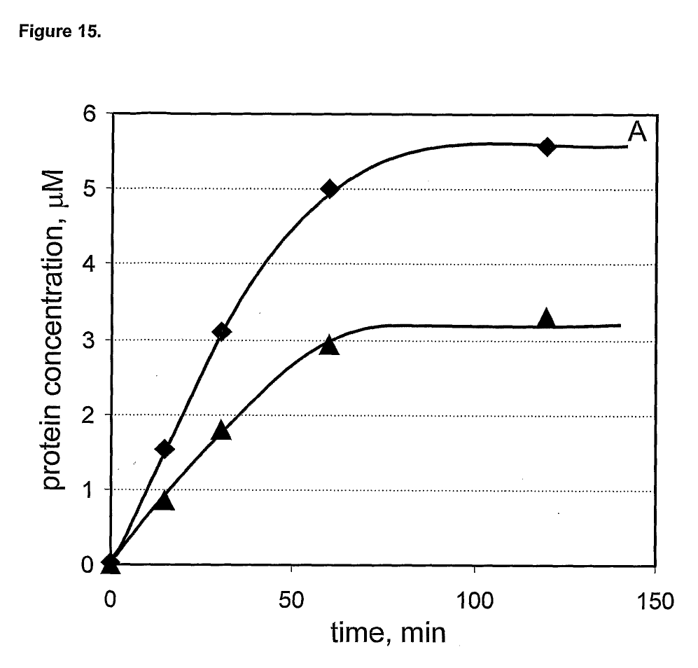

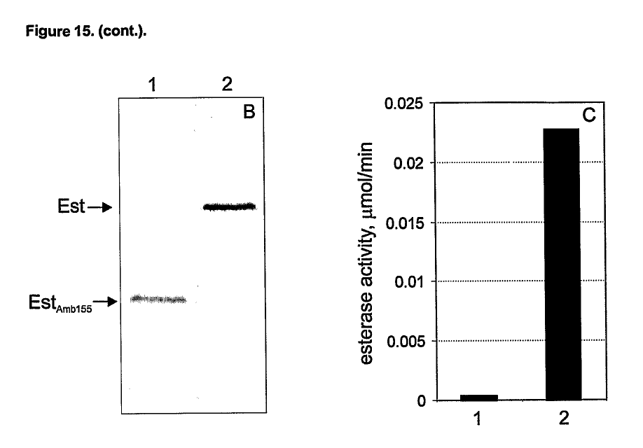

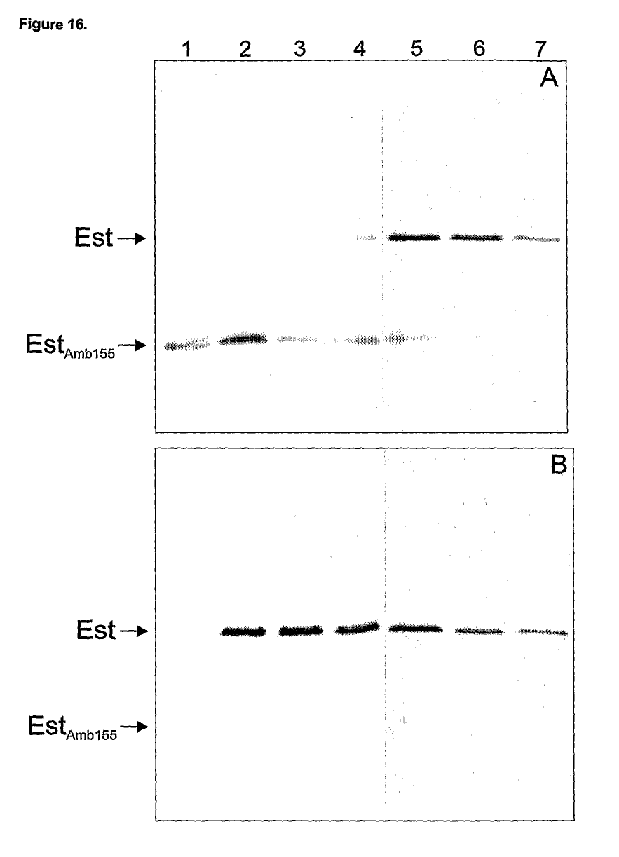

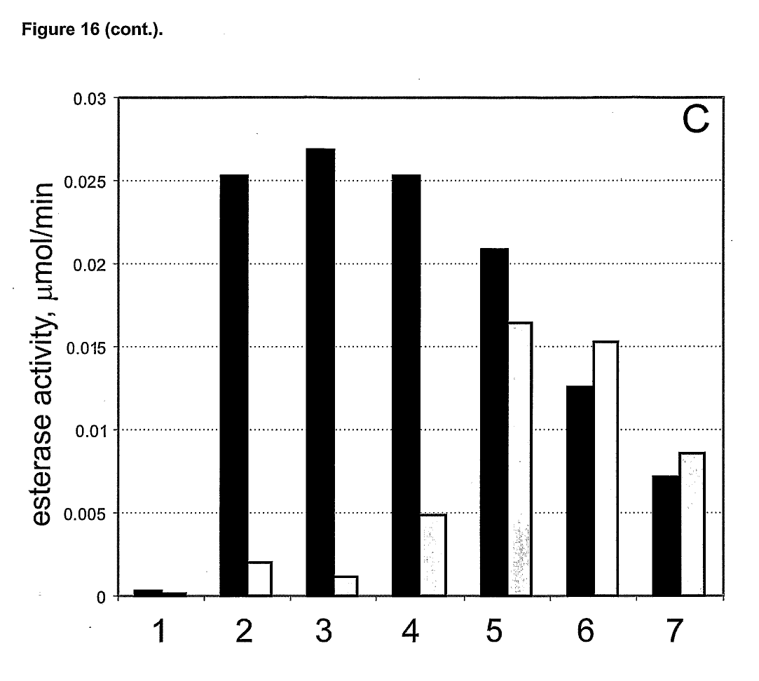

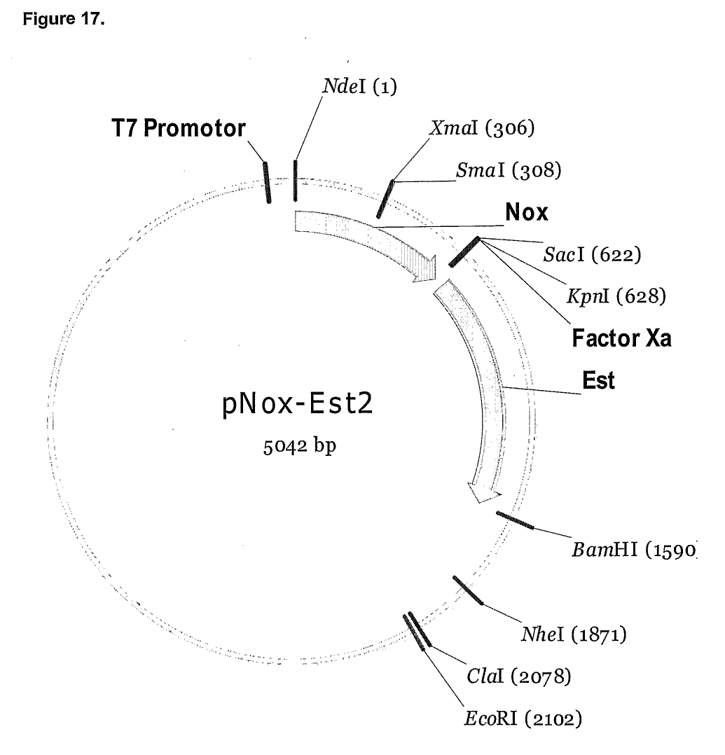

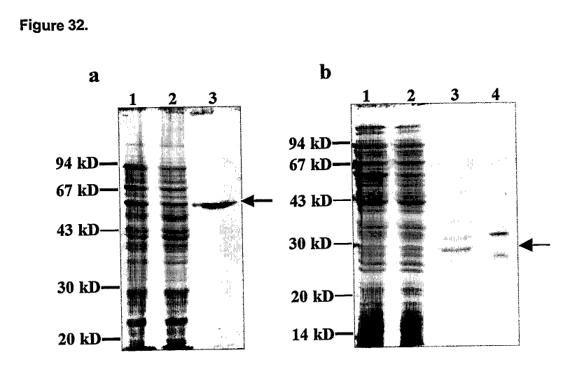

The examples further show that the esterase 2 from Alicyclobacillus acidocaldarius (Est2) can be synthesized with similar efficiency in a heterologous cell-free transcription/translation system (derived from E. coli) as an abundant homologous protein (elongation factor Ts from E. coli; FIG. 6A), even the codon usage of the esterase gene was not adjusted to the codon usage of E. coli (FIG. 6A). The synthesized esterase has high enzymatic activity (FIG. 6B) and even a 1000 fold dilution of the translation mixture which results in 10⁻⁸ M final esterase concentration provides detectable esterase-activity. The examples also document that beside standard photometric action the Est2-activity is also fluorimetrically detectable (Example 6, FIG. 6C). Further, the examples show that Est2 can be used for monitoring and/or tracking a synthesized protein (in the particular case the Est2 itself is monitored and/or tracked), in a gel after polyacrylamide gel electrophoresis (Example 6, FIG. 6C). It is further demonstrated herein that esterases, like Est2, can be used for monitoring and/or tracking a protein to be synthesized, even if the protein to be monitored and/or tracked is not the esterase itself. Said protein to be monitored and/or tracked may be (heterologously) expressed (in vitro or in vivo), and/or (affinity) purified. Examples of these applications are provided herein, e.g. by Examples 17 to 22, FIGS. 17 to 32. These non-limiting examples show the monitoring and/or tracking of the proteins NADH oxidase (Nox) from Thermus thermophilus, elongation factor Tu from Thermus thermophilus, elongation factor Ts from Thermus thermophilus, human exportin-t and putative nuclease S2001 from Sulfolobus solfataricus by Est2 during their in vitro and/or in vivo expression (Examples 17 to 21, FIGS. 17 to 31) and/or their subsequent affinity purification (Examples 22, FIG. 32). It is of particular note that a person skilled in the art is able to replace the exemplified proteins (to be monitored and/or tracked by the Est2 employed within the present application) by any other protein, polypeptide or peptide, which is desired to be monitored and/or tracked, e.g. during its (heterologous) expression (in vitro or in vivo), and/or (affinity) purification. Further, it is exemplified herein that the Est2 can be reversibly immobilized on a solid surface by binding to a specific esterase-inhibitor (trifluoromethyl-ketone (TFK)) and therefore can act as an affinity tag for affinity purification of proteins, polypeptides or peptides. After mobilizing the protein, polypeptide or peptide (X) used to Est2 (E), said X can be released from the solid surface either by releasing the fused esterase from its inhibitor or by cleaving a linker that connects Est and X (FIG. 8). In particular, it was demonstrated herein that Est2 can be used for monitoring and/or tracking enhanced fluorescent protein (eGFP) during affinity purification (Example 7, FIG. 9). Thereby, an eGFP-Est2 fusion protein was synthesized in an in vitro transcription/translation system derived from E. coli (FIG. 9, lane 1) and bound to a matrix/solid substrate (Sepharose) carrying immobilized TFK (FIG. 9, line 2). Afterwards, the linker (e.g. the linker as encoded by the nucleotide sequence of SEQ ID NO: 7) connecting eGFP and Est2 was cleaved by a factor Xa protease and the eGFP was released from the matrix (FIG. 9, line 3). Successively, the esterase 2 was also released from the immobilized TFK (FIG. 9, line 4). During all steps of synthesis and purification of the fusion protein and its components a monitoring and/or tracking of these proteins was performed by action of the esterase function (FIG. 9B). The monitoring and/or tracking by detection of the function of Est2 was validated by additional detection of an incorporated radioactively labelled amino acid ([¹⁴C]leucin) and by detection of the fluorescence of eGFP. Furthermore, the Examples show that the monitoring and/or tracking of the synthesis of an esterase (esterase-function) or a fragment thereof in a cell-free translation system can be used for detection of inhibitory or enhancing effects of substances on the function of protein biosynthesis in cell-free (coupled transcription/) translation systems. Thereby, esterase (Est2) can be used from screening of substances that influence the function of protein biosynthesis. Therefore, an esterase (Est2) is synthesized in cell-free (coupled transcription/) translation systems and the esterase activity during synthesis is detected. By adding substances to the screened to the cell-free (coupled transcription/) translation system in which the synthesis of the esterase (Est2) occurs, the effect of the added substance to protein biosynthesis can be investigated by detecting changes of the esterase activity (FIG. 10). The examples further demonstrate the usage of Est2 to monitor and/or track the synthesis of alloproteins. (Example 12, FIG. 11; Example 16, FIGS. 15 and 16). In particular it was shown that Est2 incorporates biotinylated puromycin at high yield in the presence of antibodies directed against release factor 1 of Thermus thermophilus (SEQ ID NO: 4) in a cell-free coupled transcription translation system (Example 12, FIG. 11). In the particular case, the synthesis of the Est2-puromycin-biotin conjugate was performed on strepavidin-coded glass plates and the Est2-activity was detected directly on said strepavidin-coded glass plates having immobilized the synthesized esterase-puromycin-biotin conjugate (FIG. 11, Spot 4). The examples further show the use of an esterase for monitoring and/or tracking the synthesis of a protein in a cell-free translation system from E. coli, having the release factor 1 contained in that cell-free translation system inactivated. This inactivation was achieved by addition of antibodies directed against release factor 1 from Thermus thermophilus which are capable to deplete the release factor 1 from E. coli (SEQ ID NO: 6) from the cell-free translation system by precipitation. It was demonstrated that in presence of suppressor tRNA^{SerCUA} and in the absence of release factor 1 an artificially introduced nonsense codon (replacing serine 155 of the Est2 which is essential for the function of Est2) was suppressed. Example 16, FIG. 14 to 16. In this particular case, esterase activity was only detectable when the (functional) full length Est2 was synthesized. The synthesis of the functional Est2 only takes place, when the release factor 1 from E. coli was depleted from the cell-free translation system and thereby the suppressor seryl-tRNA^{SerCUA} was able to bind to the introduced nonsense codon to deliver the essential serine 155 (FIG. 15B/C (2); FIG. 16). The esterase activity was not detectable when the active release factor 1 obviates the binding of the suppressor seryl-tRNA^{SerCUA} to the artificially introduced nonsense codon and forces the synthesis of a non-functional Est2-fragment, due to termination (FIG. 15B/C (1); FIG. 16A (1)).

As detailed above and exemplified herein, the present invention provides for the use of an enzyme, i.e. an esterase, preferably a thermostable esterase isolated or obtained from thermophilic bacteria, more preferentially from Alicyclobacillus acidocaldarius, most preferably the esterase 2 from Alicyclobacillus acidocaldarius (Est2; Manco (1998), Biochem. J., 332, 203-212) as a reporter enzyme for monitoring and/or tracking of protein synthesis in, preferably, in vitro (coupled transcription/)translation systems. However, the use as a reporter enzyme in in vivo expression systems, e.g. eukaryotic or prokaryotic cells, preferably in prokaryotic cells, is also envisaged and exemplified herein. In vivo expression systems may be prokaryotic systems, like the E. coli expression system employed in the examples appended for the expression of heterologous fusion proteins like “X-esterase” or “esterase-X” as defined herein. However, similar heterologous expression of fusion proteins (“X-esterase” or “esterase-X”) as defined herein is also envisaged in eukaryotic cells, like yeast cells, plant cells, or animal cells. These animal cells may e.g. be insect cells or mammalian cells. Corresponding expression systems (in vivo expression systems) are known in the art and comprise the expression in CHO cells, COS cells, HELA cells and the like.

The sequences coding for Est2 are known in the art and, e.g. obtainable from Hemilä (1994), Biochim. Biophys Acta 1210, 249-253. Furthermore, said sequences are documented herein under SEQ ID. No. 1 (coding sequence) and by the expressed amino acid sequence shown in SEQ ID NO. 2 or SEQ ID NO. 62. It is evident that the person skilled in the art may modify said sequences for specific purposes. For example, as also done herein, specific further/additional restriction sites may be introduced. Corresponding examples are given in the Est2 sequences comprised in the plasmids provided herein and shown in SEQ ID. NO 8, 9 or 10.

The term “isolated from” is not limited to direct isolation of said esterase from the corresponding species but relates, in particular, to its recombinant expression in the prokaryotic cell-free translation systems. As exemplified herein and shown in the appended examples, particular preferred is a cell-free translation system derived from E. coli.

Whenever employed herein, the term “cell-free translation system” is not limited to systems, wherein solely translation occurs. The term also, and in a preferred embodiment, comprises all cell-free coupled transcription/translation systems in which not only translation from a given RNA/mRNA occurs, but also the transcription step, e.g. the transcription from a given vector and/or DNA, like cDNA (peGFP-Est2 (SEQ ID NO:8)) takes place. The term “transcription” refers to the synthesis of RNA/mRNA, capable to code for the protein to be synthesized, by the cellular transcription machinery using a DNA, for example a cDNA as a template. The mode of operation and the composition of the cellular transcription machinery is known to a person skilled in the art. The term “translation” refers to the synthesis of a protein, polypeptide or peptide, whereby the transcription product (RNA/mRNA) acts as a template for the cellular translation machinery. Again, the mode of operation and the composition of the cellular translation machinery is known to a person skilled in the art.

In accordance with this invention, the term “esterase” relates to an enzyme with the enzymatic function or activity of a polypeptide (or of a fragment of such a polypeptide) which is capable of the cleavage of an ester into an alcohol and an carboxylic acid. In context of the present invention, the term “alcohol” refers to a compound carrying at least one hydroxyl group and the term “carboxylic acid” refers to a compound carrying at least one carboxyl group. Said “esterase” preferably refers to a protein comprising the consensus sequence HGGG and GXSXG as described in Hemilä ((1994), Biochemica et Biophysica kcta 1210, 249-253). Esterases are known in the art and comprise but are not limited to i.e. esterases as disclosed in Hemilä (1994), loc. cit. As detailed below and as shown in the appended examples, a particular preferred esterase to be used in context of this invention is a thermostable esterase, most preferably an esterase of prokaryotic origin. A preferred example of such an esterase is the esterase 2 from Alicyclobacillus acidocaldarius, as described herein below and as shown in (Manco, G. (1998), Biochem. J 332 (Pt 1), 203-212). The coding sequence of said esterase 2 is shown in SEQ ID NO: 1, the corresponding amino acid sequence is shown in SEQ ID NO: 2 or SEQ ID NO. 62. It is preferred that the amino acid sequence of the esterase 2 from Alicyclobacillus acidocaldarius to be employed within the present invention is that of SEQ ID NO. 62.

The term “esterase” as employed herein does not only comprise full-length esterases, but also functional fragments of esterases which are capable of the cleavage of an ester into an alcohol and an carboxylic acid. Such a “functional fragment” may be of any length, however, preferably such functional fragments comprise at least 50, more preferably at least 60, more preferably at least 80 and more preferably at least 100 amino acid residues.

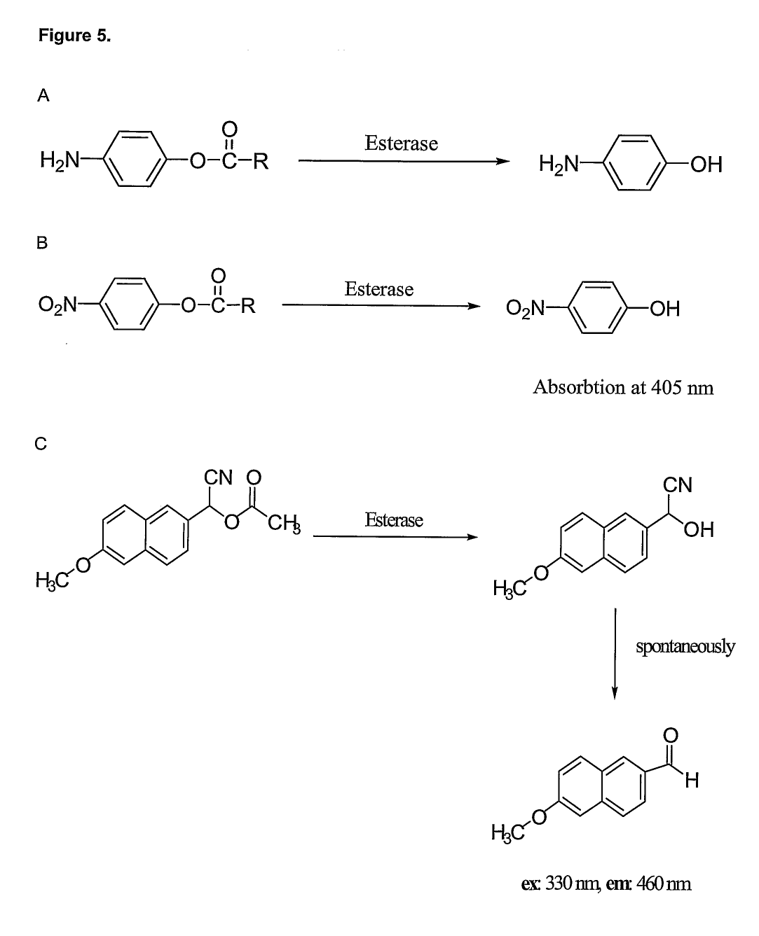

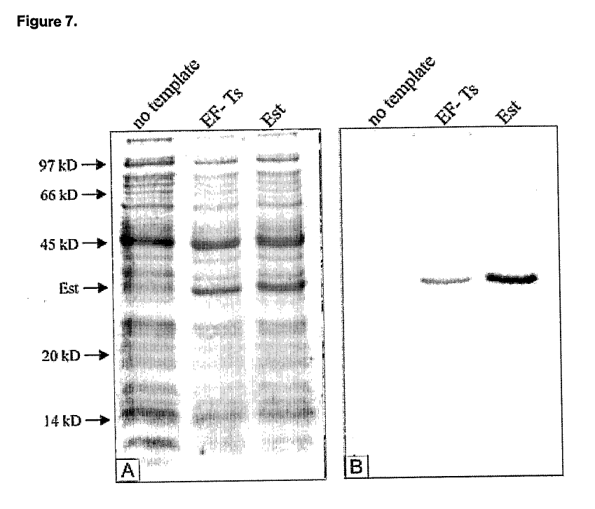

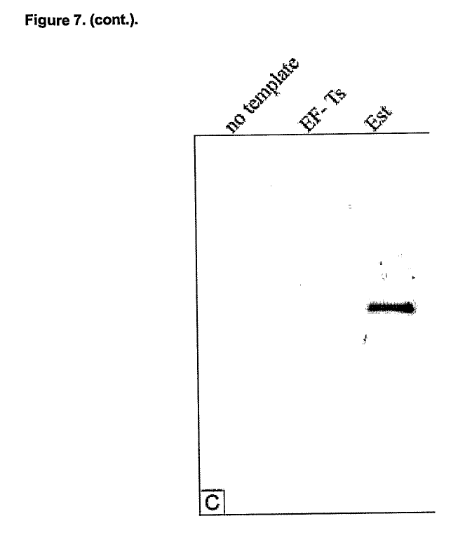

In context of the invention, particular preferred esterases are single chain esterases. However, it is also envisaged that individual, single chained polypeptides are employed in context of this invention which are derived from bi- or multichained esterases or from (esterase) complexes. These single chained polypeptides to be employed in context of this invention comprise the esterase activity or at least a part of said activity. Accordingly, as used herein, the term “esterase” relates to any polypeptide which can be expressed in cell-free translation systems and which comprise an esterase activity which may be measured. The measurement of “esterase activity” is performed by methods known in the art and as detailed herein, in particular in the appended examples. Such methods for the detection of “esterase activity” comprise photometric detection, wherein e.g. the esterase-catalysed hydrolysis of p-nitrophenyl acetate to the corresponding alcohol is performed (see, inter alia, FIG. 5B, FIG. 6B, example 6), electrochemical detection, wherein e.g. the esterase-catalysed hydrolysis of p-aminophenyl acetate to the corresponding alcohol is performed (see, inter alia, FIG. 5A) and fluorescent detection (see, inter alia, FIG. 5C; example 6, FIG. 6C), wherein e.g. the esterase hydrolyses 5-(and -6)-carboxy-2′,7′-dichlorofluorescein diacetate which leads to the appearance of a fluorescent product. Furthermore it is exemplified herein that the detection of an esterase is also possible in the gels after sodium dodecyl sulfate polyacrylamide gel electrophoresis; see, inter alia, FIG. 7C.

As pointed out above, a particular preferred esterase of the present invention is the esterase 2 of Alicyclobacillus acidocaldarius. However, besides prokaryotic also eukaryotic esterases (or functional fragments thereof) may be employed in the uses and methods described herein.

The preferred esterase (Est2) from Alicyclobacillus acidocaldarius to be employed in the inventive uses and methods is a thermostable enzyme that consists of one polypeptide chain and possesses a broad substrate specificity (Manco, G. (1998) loc. cit.). Due to high thermostability, practically instant folding and refolding and easily detectable activity, this esterase has a potential application as a reporter for in vitro and in vivo protein expression systems. The tertiary structure of the esterase was determined by X-ray crystallography (De Simone, G. (2000) J Mol. Biol 303, 761-771). Serine 155, located in the Ser-His-Asp catalytic triad (FIG. 14B), is essential for hydrolytic activity (De Simone, G. (2000) J Mol. Biol 303, 761-771) It is encoded by the ACG triplet at the corresponding position of the est2 mRNA (Hemila (1994), Biochim. Biophys. Acta 1210, 249-253). As already mentioned before and exemplified herein below, the coding sequence for serine 155 was substituted to a RF1-dependent stop codon (UAG) and the resulting construct was used to test the conditions for efficient termination and/or suppression at UAG stop codon.

Many investigators dealing with mechanism of termination and suppression of termination codons use SDS-PAGE as a criterion for monitoring of suppression events. The possibility of increased translation error rates due to high concentrations of unnatural suppressor tRNAs were usually disregarded. The construction of Est2 mRNA (amber 155) from the template pEst2_amber 155 (see herein below) allows to monitor in parallel the efficiency of the UAG suppression by a band shift in SDS-PAGE and the accumulation of esterase activity in the in vitro translation mixture. This assay is suitable for estimation of optimal conditions to achieve highly efficient suppression in different in vitro translation mixtures. Such assessment seems to be very important since translation systems may individually differ from each other due to different source and preparation method.

The 34 kD esterase described herein is a thermostable, single chain protein that folds into a one domain structure with one active center that possess a lipase-like Ser-His-Asp catalytic triad (De Simone, (2000) J. Mol. Biol, 303, 761-771.). The overall fold, typical for α/β hydrolases, shows a central eight-stranded mixed β-sheet surrounded by five helices with a helical cap on a top of the C-terminal end of the central β-sheet. The N and C-terminal ends of the protein are not involved in catalytic center of the enzyme and are exposed on the esterase surface (De Simone (2000) loc. cit.) providing a possibility for the protein to be fused with other polypeptides without altering the esterase native fold.

Esterase 2 from thermophilic bacteria Alicyclobacillus acidocaldarius can easily be produced up to 200 μg/ml by coupled in vitro transcription/translation system derived from E. coli, without any codon usage adjustment and keeping its activity. The activity of the produced esterase can be monitored directly in the translation mixture. Accordingly, this is an example how an esterase can successfully be employed for monitoring/tracking of biosynthesis. The examples provided herein in context of esterase 2 apply, mutatis mutandis, for other esterases. The photometric assay presented in FIG. 5B allows the detection of 10-12 moles of esterase/esterase activity in 100 μl assay volume. Use of micro plates allows to increase this detection limit by a factor of 10 to 100, reaching the sensitivity comparable with radioisotope labeling. The utilization of carboxyfluorescein diacetates as the esterase substrates allows fluorescent detection (FIG. 6C) that can be used for various applications in cell biology, biochemistry as well as pharmaceutical research. For example a fusion of the esterase with polypeptides allows the cellular localization by confocal microscopy. A remarkable feature of the esterase 2 from Alicyclobacillus acidocaldarius is its fast folding into a stable, active, single domain structure allowing refolding and detection of the esterase activity in polyacrylamide gels after SDS electrophoresis and removal of the SDS. The sensitivity of this activity detection is well-comparable with the sensitivity of the detection of 14C-labelled proteins by autoradiography (FIG. 7). Therefore, within the scope of the present invention is also the monitoring/tracking of protein biosynthesis with gel-technology, i.e. 2D-gels or even 3D-gels as well as gel-transfer technologies. However, as detailed in the appended examples, and described above, also a simple detection system on the gel per se is provided. Stability of esterase enzymes, like esterase 2 at wide temperature range (10-75° C.) and activity over a broad pH (5-8) allows the use of heat or acidic precipitation as a simple and rapid purification step for successive isolation of the esterase fused proteins.

A preferred esterase to be used, in context of this invention is the esterase 2 described above and esterase/esterase activity which are homologous to said esterase. Accordingly, the esterase/esterase activity to be employed in the uses and methods provided herein may be selected form the group consisting of:
(a) an esterase encoded by a nucleotide sequence comprising a nucleotide sequence as shown in SEQ ID NO: 1;
(b) an esterase encoded by a nucleotide sequence coding for a polypeptide comprising an amino acid sequence as shown in SEQ ID NO: 2 or 62;
(c) an esterase encoded by a nucleotide sequence of a nucleic acid molecule that hybridizes to the complement strand of a nucleic acid molecule comprising an nucleotide sequence as defined in (a) or (b) and which catalyses the cleavage of an ester into an alcohol and an carboxylic acid;
(d) an esterase which comprises an amino acid sequence as shown in SEQ ID NO: 2 or 62;
(e) an esterase which comprises an amino acid sequence which is at least 60% identical to the full length amino acid sequence as shown in SEQ ID NOS: 2 or 62; and
(f) an esterase encoded by a nucleotide sequence which is degenerated to a nucleotide sequence as defined in any one of (a) to (c).

SEQ ID NO: 1 refers to the coding nucleotide sequence of esterase 2 from Alicyclobacillus acidocaldarius as described in Manco ((1998) loc. cit.). SEQ ID NOs: 2 or 62 refer to the amino acid sequence of the esterase 2 from Alicyclobacillus acidocaldarius. Within SEQ ID NO: 2, the internal methionine residues (Met (M)), encoded by their corresponding nucleotide residues of SEQ ID NO: 1, are indicated as X.

In context of the present invention, the term “nucleic acid(s)” and/or “nucleic acid molecule(s)” encompasses all forms of naturally occurring types of nucleic acid(s) and/or nucleic acid molecules as well chemically synthesized nucleic acids and also encompasses nucleic acid analogs and nucleic acid derivatives such as e.g. locked DNA, PNA, oligonucleotide thiophosphates and substituted ribo-oligonucleotides. Furthermore, the term “nucleic acid” and/or “nucleic acid molecules(s)” also refers to any molecule that comprises nucleotides or nucleotide analogs.

Preferably, the term “nucleic acid(s)” and/or “nucleic acid molecule(s)” refers to oligonucleotides or polynucleotides, including deoxyribonucleic acid (DNA) and ribonucleic acid (RNA). The “nucleic acids” and/or “nucleic acid molecule(s)” may be made by synthetic chemical methodology known to one of ordinary skill in the art, or by the use of recombinant technology, or may be isolated from natural sources, or by a combination thereof. The DNA and RNA may optionally comprise unnatural nucleotides and may be single or double stranded. “Nucleic acid(s)” and/or “nucleic acid molecule(s)” also refers to sense and anti-sense DNA and RNA, that is, a nucleotide sequence which is complementary to a specific sequence of nucleotides in DNA and/or RNA.

Furthermore, the term “nucleic acid(s)” and/or “nucleic acid molecule(s)” may refer to DNA or RNA or hybrids thereof or any modification thereof that is known in the state of the art (see, e.g., U.S. Pat. No. 5,525,711, U.S. Pat. No. 4,711,955, U.S. Pat. No. 5,792,608 or EP 302175 for examples of modifications). Such nucleic acid molecule(s) are single- or double-stranded, linear or circular, natural or synthetic, and without any size limitation. For instance, the nucleic acid molecule(s) may be genomic DNA, cDNA, mRNA, antisense RNA, ribozyme or a DNA encoding such RNAs or chimeroplasts. Preferably, said nucleic acid molecule(s) is/are in the form of a plasmid or of viral DNA or RNA. Nucleic acid molecule(s) may also be oligonucleotide(s), wherein any of the state of the art modifications such as phosphothioates or peptide nucleic acids (PNA) are included.

In the context of the present invention the term “hybridizes” refers to hybridization under conventional hybridization conditions, preferably under stringent conditions, as for instance described in Sambrook and Russell (2001), Molecular Cloning: A Laboratory Manual, CSH Press, Cold Spring Harbor, N.Y., USA. In an especially preferred embodiment, the term “hybridizes” refers to hybridization that occurs under the following conditions: Hybridization buffer: 2×SSC; 10×Denhardt solution (Fikoll 400+PEG+BSA; ratio 1:1:1); 0.1% SDS; 5 mM EDTA; 50 mM Na2HP04; 250, ug/ml of herring sperm DNA; 50 ug/ml of tRNA; or 0.25 M of sodium phosphate buffer, pH 7.2; 1 mM EDTA 7% SDS Hybridization temperature T=60° C. Washing buffer: 2×SSC; 0.1% SDS Washing temperature T=60° C. Polynucleotides which hybridize to the complement strand of a nucleic acid molecule, comprising a nucleotide sequence as defined herein, can, in principle, encode a polypeptide having esterase activity from any organism expressing such polypeptides or can encode modified versions thereof. Polynucleotides which hybridize with the polynucleotides as defined in connection with the invention can for instance be isolated from genomic libraries or cDNA libraries of bacteria, fungi, plants or animals. Preferably, such polynucleotides are of procaryotic origin, particularly preferred from Alicyclobacillus acidocaldarius. Furthermore, the esterase contained in said cell-free translation system may also be at least 50%, more preferably at least 60%, more preferably at least 70%, more preferably at least 80%, even more preferably at least 90% and most preferably at least 95% identical to the full length amino acid sequences as shown in SEQ ID NO: 2 or 62.

The esterase (esterase activity) is normally, in context of this invention, used in form of one part of a fusionprotein, fusionpolypeptide or fusionpeptide, as detailed herein. Accordingly, in a most preferred embodiment of the invention, a use of an esterase in monitoring/tracking the synthesis of a desired protein/polypeptide/peptide is described, whereby said esterase is covalently attached/bound/fused to said protein/polypeptide/peptide. Said covalent attachment/binding/fusion may, on the protein level, be at the N- as well as at the C-terminus of the protein/polypeptide/peptide the expression/synthesis or which is to be monitored. Accordingly, and as further described below and illustrated in the appended examples, the use of the esterase as described herein is in particular envisaged in the provision of nucleic acid molecules (e.g. DNA, RNA, vectors and the like) wherein nucleic acid molecule is provided which comprises a coding sequence for an esterase (or an esterase activity) or a functional fragment thereof and a (further) coding sequence for the protein/polypeptide/peptide who's expression/synthesis in the in vitro system is to be monitored/tracked in accordance with this invention. Said nucleic acid molecule, coding for an esterase (esterase activity) and the desired protein/polypeptide/peptide (also denoted herein as “X” or “X′”) is than expressed in the cell-free system as described herein. Accordingly, the coding sequence of X/X′ is in frame with the coding sequence of said esterase/esterase activity or said functional fragment of the esterase/esterase activity. The nucleic acid sequence, therefore, codes for an esterase-X/X-esterase fusion construct as detailed herein. Further embodiments on corresponding nucleic acid molecules as well as vectors are provided herein below.

The term “monitoring and/or tracking the synthesis of a protein, polypeptide or peptide” means that, inter alia, changes of the amount of a protein, polypeptide or peptide, in particular the increase of the amount of protein, polypeptide or peptide can be detected and quantitatively and/or qualitatively determined, during, before and/or after the synthesis of said protein, polypeptide or peptide. The corresponding detection of changes is carried out by measuring the esterase/esterase activity as described herein. Further, the term “monitoring and/or tracking the synthesis of a protein, polypeptide or peptide” means that the efficacy of the synthesis of a protein, polypeptide or peptide can be determined. The term also means that the synthesis rate of the protein, polypeptide or peptide can be determined, in particular in the absence or presence of inhibitors or enhancers of protein biosynthesis. Accordingly, with the systems and methods provided herein, also inhibitors or activators of translation systems may be determined. The term “monitoring and/or tracking the synthesis of a protein, polypeptide or peptide” also relates to the determination of the functionality of the protein, polypeptide or peptide can be determined during, before and/or after its synthesis. “Functionality” refers to the ability of the protein, polypeptide or peptide to exert its function and/or activity. In particular, the term “determining the functionality” of a protein, polypeptide or peptide means that it is determined (quantitatively or qualitatively) to what extent, the protein, polypeptide or peptide exerts its function and/or activity. The term “function” also relates to a physiological function within an organism. The term “monitoring and/or tracking the synthesis of a protein, polypeptide or peptide” also means that a protein, polypeptide or peptide can be localized during, before and/or after the synthesis of said protein, polypeptide or peptide. “Localization” of a protein, polypeptide or peptide means that a particular place, where a certain amount of said protein, polypeptide or peptide exists, is identified. This particular place may be, but is not limited to, in form of a vial, a gel, a blot, a column, a membrane, a slide (e.g. out of glass, polystyrene etc.) a liquid, a droplet, a cell, a tissue, beads and the like. However, said “localization”, as described above may also comprise the localization of a protein in a cellular context, for example whereby said esterase is detected within the context of a synthesized esterase-fusion protein/-fusion construct in a cell. The term “localization” in this context also comprises, inter alia, the detection of the presence of the esterase moiety of the fusion construct described herein. Corresponding examples relate, inter alia, to the transfection of a cell with a vector described herein and the detection of a synthesized esterase-fusionprotein/-fusion construct, for example by microscopical means.

All the above recited “monitoring/tracking steps” are, in accordance with the present invention, based on the detection of a specific activity of an esterase, i.e. the esterase activity, more preferably an Est2 activity. Corresponding embodiments are clearly evident from this specification as well as from the appended examples.

Yet, it is envisaged that not only the function of esterase or esterase activity, in particular in context of the herein described fusion constructs “X-esterase” or “esterase-X” (and the like) be detected in order to carry out the monitoring step and/or tracking step provided herein. Said monitoring and/or tracking step may also comprise the detection of the presence of said esterase, e.g. by immunological means, like microscopical techniques or immunolocalisation methods, like, inter alia, Western blots. Furthermore, the detection via radioactive labels (and the like) of the presence of the esterase moiety in the or on the fusion constructs (comprising at least one of the proteins, polypeptides or peptides desired to be synthesized (or synthesized) in the herein described cell-free systems and the moiety comprising the esterase and/or esterase activity) is envisaged. Examples for the detection of the function and/or the presence of esterase or esterase activity are provided herein and in the appended examples.

The use of an esterase as described herein, has several advantages compared to the use of the reporters in the prior art:

First, esterases, in particular the esterase 2 from Alicyclobacillus acidocaldarius, are stable and active over a wide range of temperature (10-75° C.) and pH values (pH 5-8).

It was surprisingly found and exemplified herein that especially the detection of products of esterases-catalysed reactions are very sensitive. The examples show that even at concentrations of, for example, ˜10⁻⁸ M, a detection of this product is still possible (example 6, FIG. 6). Therefore, the use of esterases, in particular the esterase 2 from Alicyclobacillus acidocaldarius provides for a highly sensitive monitoring and/or tracking of protein synthesis, in particular in cell-free translation systems. For example, this detection might be performed by photometric, electrochemical or fluorescent methods, as exemplified herein. In an example for photometric detection, the esterase-catalysed hydrolysis of p-nitrophenyl acetate to the corresponding alcohol is performed (see, inter alia, FIG. 5B, FIG. 6B, example 6). In an example for electrochemical detection, the esterase-catalysed hydrolysis of p-aminophenyl acetate to the corresponding alcohol is performed (see, inter alia, FIG. 5A). In an example for fluorescent the esterase hydrolyses 5-(and -6)-carboxy-2′,7′-dichlorofluorescein diacetate which leads to the appearance of a fluorescent product (FIG. 6C, example 6).

Furthermore it is exemplified herein that the detection of an esterase might also be possible in gels after sodium dodecyl sulfate polyacrylamide gel electrophoresis (PAGE; see, inter alia, FIG. 7C, example 6). Furthermore, the use of esterases, in particular of the esterase 2 from Alicyclobacillus acidocaldarius (˜34.4 kD), benefits from the fact, that esterases have normally low molecular masses and a fast folding, globular, single domain structure having a very high enzymatic activity, in particular in cell-free translation systems as shown herein.

In addition, a maturation of the esterases, in particular of the esterase 2 from Alicyclobacillus acidocaldarius, is not required. This is in stark contrast to other reporter proteins, like, inter alia, GFP.

Moreover, it was also surprisingly found that immobilization of inhibitors of esterases allow affinity purification of the synthesized esterase either alone or of a protein, polypeptide or peptide fused to an esterase (see, e.g. example 7). This purification can occur in particular in the format of fusion constructs in the herein described form at “X-esterase” or “esterase-X”, wherein said esterase interacts with an immobilized binding partner of the esterase (for example an inhibitor, like trifluoromethyl-alkylketones; FIG. 8(1), example 7, FIG. 9). After binding of the esterase part of the fusionprotein with its binding partner (FIG. 8(2)), as described herein (example 7, FIG. 9), the esterase fusionprotein can be either released from its binding partner as a whole (FIG. 8(3a)) or the desired protein, polypeptide or peptide may be cleaved of from the esterase part. This may be carried out for example, and as described herein, by cleaving an introduced linker between the esterase and the desired protein (FIG. 8(3b), example 7, FIG. 9). Therefore, present invention provides for an vector comprising the coding sequence of an esterase, preferably esterase 2 from Alicyclobacillus acidocaldarius, and a part X, wherein said part X is a protein, polypeptide or peptide to be purified. Preferably, the esterase and the part X that are comprised in the vector of the invention (as described below), are connected (in frame) via a linker, preferably cleavable by proteases, more preferably cleavable by a factor XA protease. As shown in the appended examples, the vector of the present invention may be used as a template for in vitro synthesis of the encoded esterase fusionprotein. The resulting translation mixture, containing the synthesized esterase fusionprotein may be incubated in presence of a matrix (i.e, Sepharose CL-6B) carrying an immobilized esterase-binding partner, preferably an esterase inhibitor, (i.e, trifluoromethyl-alkylketones (TFK)). After binding of the esterase fusionprotein to the binding partner, the components of the esterase fusionprotein may be released as described above and exemplified herein (example 7, FIG. 9). The matrix, the binding partner of the esterase part is immobilised on, may be in form of beads, columns, flat surfaces (i.e. glass slides), coated vials and the like. A skilled person can easily substitute the matrix as exemplified herein (example 7, FIG. 9) with any desired matrix to be used for immobilising the esterase binding partner. Thus, the present invention also provides for the use of an esterase as a reporter enzyme to monitor and/or track the synthesis of proteins, polypeptides or peptides fused to said esterase, preferably to the N-terminus of said esterase, and also as a cleavable tag for the purification of said proteins, polypeptides or peptides. Further illustrative details are given in the appended examples.

In summary, it is demonstrated herein and in particular in the appended examples that the presence of an esterase at the N- or C-terminus of a protein, polypeptide or peptide allows the use of said esterase for concomitant purification and detection of proteins, polypeptides or peptides, especially in the corresponding generation of these proteins, polypeptides or peptides in in vitro translation systems. As demonstrated herein, the esterase can be fused with a target protein, polypeptide or peptide resulting in a product that possesses activities and/or features of esterase as well as the additional, conjugated proteins, polypeptides or peptides. The additional activity may be, but is not limited to, an enzymatic, hormonal, signal activity and the like. However, said additional activity and/or feature may also comprise the marker and/or structural function of the (additional) conjugated protein, polypeptide or peptide. Therefore the corresponding (additional) function of the herein described fusion construct/fusion protein corresponds to the function or activity of the gene product (or a fragment thereof) as encoded by the target gene to be expressed. The presence of the esterase on the N- or C-terminus of the target protein, polypeptide or peptide permits quantitative determination of expression levels by esterase activity measurement, since esterase translation is possible after completion of a target gene. This could be used for a rapid optimization of in vitro or in vivo expression conditions. Furthermore, the (recombinant) proteins, polypeptides or peptides can be one step isolated by esterase inhibitor based affinity chromatography. The esterase to be employed herein, e.g. the esterase from Alicyclobacillus acidocaldarius, combines the properties of a robust and sensitive reporter protein along with a high affinity binding tag in one molecule.

The esterases to be employed in context of this invention, in particular Est2, can be in vitro synthesized by (coupled transcription/)translation using a corresponding plasmid. An adjustment of the codon usage is not always required for in vivo as well as in vitro synthesis. Accordingly, the esterases, in particular Est2, as described herein, can be employed preferably in a prokaryotic system, more preferably in a cell free prokaryotic system and most preferably in a cell free system from E. coli. However, also the use of esterases, in accordance with this invention, in in vivo expression systems, like E. coli systems, is envisaged.

As pointed out above, the esterase part can also be immobilized, inter alia, on solid surfaces by linking the esterase with affinity tags such as oligonucleotides, biotin, chemically and photochemically reactive groups and/or chemical structures that do not occur in natural polypeptides, for example, using puromycin-termination technology (Nemoto (1999), FEBS Lett. 462, 43-6; see, inter alia, example 12, FIG. 11).

Moreover, the use of esterases as provided herein also provides for a reporter group for screening protein biosynthesis-inhibitory activity in combinatorial libraries and physiological extracts. Again, details are also provided in the examples and, inter alia, FIG. 10. It is obviously evident for a person skilled in the art that not only the distinct disclosed esterases, like the Est2, can be used in context of the invention, but also other esterases, in particular members of the class of carboxylesterases. Known carboxylesterases are and comprise i.e. the carboxylesterases as disclosed in Hemilä ((1994), loc. cit.

As pointed out above, the esterase/esterase activity is preferably used in the monitoring/tracking of protein-, polypeptide-, or peptide synthesis in vitro, in particular in cell-free systems for translation. However, as detailed above, the inventive use of esterase/esterase activity is also envisaged in the context of cellular expression. Said cellular expression may take place in eukaryotic as well as prokaryotic host cells transfected with recombinant constructs capable of expressing fusion constructs/fusion proteins (“X-esterase”/“esterase-X”) as defined and exemplified herein.

Cell-free translation systems are well known in the art and the uses and methods provided herein can readily be employed in such systems. Known “in vitro” translation systems comprise, but are not limited to “in vitro” translation systems from prokaryotic cells such as E. coli cells (e.g. Zubay (1973), Imm. Rev. Genet. Vol. 7, page 267) and from eukaryotic cells such as rabbit reticulocytes (e.g. Pelham (1976), Eur. J. Biochem. Vol. 131, page 289) and wheat germ cells (e.g. Spirin (1990), American Society for Microbiology, 56-70; Endo (1992), J. Biotechnol., 25, 221-230; Stiege (1995), J. Biotechnol. 41:81-90). More details on preferred “cell-free translation systems are provided herein below and in the appended examples. Preferred in vitro translation systems in context of the inventive uses and methods provided herein are systems in which transcription and translation can occur, i.e. cell-free coupled transcription/translation systems.

Yet, such systems may be of prokaryotic or eukaryotic origin or may even be a mixture of cell-free translation systems. In context of this invention the esterase/esterase activity is preferably used in monitoring/tracking of protein expression in prokaryotic systems. As shown in the appended examples, the system and methods provided herein work particularly well in cell-free translation systems of E. coli origin. However, also translation systems like wheat germ extract cell-free translation systems or rabbit reticulocyte lysates may readily be employed in context of this invention.

The cell-free translation system, to be employed within the present invention may comprise:
a cell-free extract;
ribonucleotide triphosphates, like ATP, CTP, GTP, UTP, etc.;
a RNA polymerase;
magnesium ions; and/or
a template plasmid.

The cell-free system also may comprise additional amino acids, for example labelled amino acids or unnatural amino acids. Also comprised may be (additional) tRNA, like suppressor seryl-tRNA^{SER(CUA)}. Also, as shown below, (additional) leucine may be comprised, preferably labelled leucine. Preferably, the magnesium ions comprised in the cell-free translation system to be employed in context of this invention are at a concentration at which RNA is transcribed from DNA and RNA translates into protein. More preferably, the magnesium ions are in form of MgCl₂, e.g. at a concentration of 9-12 mM.

Preferably, the cell-free coupled transcription/translation systems, as employed in context of this invention, may comprise the ingredients as listed below:
30 S cell-free extract from E. coli (enzyme- and und ribosomal fraction);
MgCl₂ 9-12 mM;
DTT 10 mM;
Amino acids, 200 μM each (For labelling, each amino acid can be applied as a 14C amino acid with a concentration of 100 μM (e.g. 14C-leucine))
Rifampicin 0.02 mg/ml reaction mixture,
Bulk-tRNA 600 μg/ml reaction mixture,
ATP, CTP, GTP, UTP, 1 mM each,
Phosphoenolpyruvate 10 mM;
Acetylphosphate 10 mM;
Pyruvatekinase 8 μg/ml reaction mixture;
Plasmid 2 pmol/ml reaction mixture;
T7 Polymerase 500 Units/ml reaction mixture;
HEPES pH 7.6, 50 mM;
Potassium acetate 70 mM;
Ammonium chloride 30 mM;
EDTA pH 8.0, 0.1 mM;
Sodium azide 0.02%;
Polyethyleneglycol 4000 2%;
Protease inhibitors: aprotinin 10 μg/ml reaction mixture, leupeptin 5 μg/ml reaction mixture, pepstatin 5 μg/ml reaction mixture; and
Folic acid 50 μg/ml reaction mixture.

The above recited cell-free coupled transcription/translation system is merely an illustrative example of a cell-free system to be employed in context of this invention. Corresponding examples are also given in the experimental part.

Generally, the composition of cell-free translation systems, in particular cell-free coupled transcription/translation systems is well known in the art. Said systems are also commercially available, e.g., from Promega GmbH. Most preferably, and also shown in the experimental part, said cell-free coupled transcription/translation systems may be comprised in evaluation size transcription/translation kits purchased from RiNA GmbH (Berlin, Germany).

The cell-free translation systems to be employed in context of the present invention may (further) comprise a labelled amino acid. By incorporation of said labelled amino acid, it is possible to monitor and/or track the synthesis of a protein or to identify the location (e.g. in a polyacrylamide gel) of said protein. Preferably, the labelled amino acid is a radioactively labelled amino acid, more preferably the labelled amino acid is [¹⁴C]leucine, [¹⁴C]valine and/or [¹⁴C]isoleucine, most preferably the labelled amino acid is [¹⁴C]leucine.

The cell-free translation system as employed in the present invention, may also be of eukaryotic origin. In this case, a wheat germ extract cell-free translation system or a rabbit reticulocyte lysate cell-free translation system would be preferred, but a cell-free translation system based on lysates from oocytes or eggs (e.g. oocytes from Xenopus) may be also applicable. These eukaryotic systems may preferably be used for the expression of eukaryotic genes or mRNA and are also well known in the art.

Another embodiment of the cell-free translation system as employed in the present invention refers to a cell-free translation system that comprises a nonsense codon suppressing agent. Also comprised may be an inhibitor of release factors, like an anti-release factor antibody which precipitates and/or crosslinks a release factor in said cell-free translation system. Other inhibitors of release factors are known in the art and comprise, inter alia, aptamers directed against release factors; Szkaradkiewicz (2002) FEBS ltrs 514, 90-95. Also thermo-sensitive release factors have been employed in this context and are also envisaged in context of this invention; see, inter alia, Short (1999) Biochem. 38, 8808-8819.

The term “nonsense-codon suppressing agent” as used herein relates to an agent that is capable to bind to the A-site of a ribosome programmed by a stop codon. Said stop codons are known in the art and may be UAA, UAG or UGA, preferably, UAG. The nonsense-codon suppressing agent itself may be covalently bound to the elongating peptide-chain or may be delivering a substance that is bound to the elongating peptide chain. Said nonsense-codon suppressing agent may prevent normal termination accomplished by release factors or termination factors or said nonsense-codon suppressing agents may replace normal termination accomplished by release factors or termination factors. Preferably, said nonsense-codon suppressing agent that delivers a substance to be bound to the elongating peptide-chain prevents normal termination. Said nonsense-codon suppressing agent, being itself covalently bound to the elongating peptide-chain, replaces normal termination.

The nonsense-codon suppressing agent, delivering the substance to be bound to the polypeptide-chain, may be a aminoacyl-tRNA, preferably a suppressor aminoacyl-tRNA, more preferably a suppressor aminoacyl-tRNA^{(CUA)}. The nonsense-codon suppressing agent to be covalently bound to the polypeptide chain may be, inter alia and preferably, puromycine or a derivative thereof as defined herein below.

As discussed above, the cell-free system may also comprise, if desired, a component which is capable of inhibiting and/or negatively interfering with a release factor comprised in said cell-free translation system. Such a cell-free translation system is particularly useful when alloproteins (as detailed below) are desired to be synthesized in said cell-free system

The term “anti-RF antibody”, in particular “anti-RF1 antibody” as employed herein refers to an antibody, a plurality of antibodies and/or a serum comprising such antibodies which is/are able to specifically bind to, interact with and/or detect RFs, preferably RF1, more preferably RF1 from E. coli or a fragment thereof. In context of the present invention, said “anti-RF antibody” must be capable of precipitating (in the in vitro system) the RF and/or must be capable of crosslinking said RF. The “precipitation” and/or crosslinking” leads to an inactivation of the RF, inter alia, due to the formation of larger RF-antibody complexes. The term “precipitates and/or crosslinks”, accordingly, refers to the capability of an anti-release factor antibody to bind and to inactivate a release factor. Therefore, said binding leads to an inactivation of said release factors which is equivalent of a depletion of said release factor (from cell-free translation systems). The term “inactivation” refers to making said release factors incapable to bind to the A-site of the ribosome and thereby incapable to cause termination of the peptide-chain and its release from the ribosomal complex. The precipitating and/or deactivating activity of anti-RF polyclonal antibodies can be measured by the residual RF activity in the in vitro translation system, by testing the hydrolysis of a peptide from peptidyl-tRNA located in the P-site (Freistroffer (2000), Proc Natl Acad Sci USA. 97, 2046-51). or by a gel electrophoresis followed by Western blotting, which is being a common laboratory praxis.

Corresponding antibodies directed against an release factor may easily be prepared as demonstrated in the appended examples and as known in the art. Said antibodies and/or sera may, inter alia, be prepared by immunization of a non-human vertebrate with purified and/or recombinantly produced “release factors”. In the appended examples, it is documented how, for example a polyclonal serum against release factor 1 (RF1) of Thermus thermophilus (T. th.; SEQ ID NO: 4) can be prepared. In the corresponding example, a heterologuesly expressed, recombinantly produced RF1 was used in the immunization protocol. The preparation of antibodies, either monoclonal or polyclonal, is well known in the art; see, inter alia Harlow/Lane (“Antibodies: A laboratory manual” (1988), CSHL, New York).

The person skilled in the art readily in the position to deduce whether an antibody and/or antibody molecule or a serum directed against a given release factor is capable of precipitating and/or crosslinking said release factor.

The term “anti-RF antibody” also relates to a serum, in particular a purified serum, i.e. a purified polyclonal serum. The antibody molecule is preferably a full immunoglobulin, like an IgG, IgA, IgM, IgD, IgE, IgY (for example in yolk derived antibodies). The term “antibody” as used in this context of this invention also relates to a mixture of individual immunoglobulins. Furthermore, it is envisaged that the antibody/antibody molecule is a fragment of an antibody, like an F(ab), F(abc), Fv Fab′ or F(ab)₂. Furthermore, the term “antibody” as employed in the invention also relates to derivatives of the antibodies which display the same specificity as the described antibodies. Such derivatives may, inter alia, comprise chimeric antibodies or single-chain constructs. Yet, most preferably, and as shown in the examples, said “anti-RF antibody” relates to a serum. Also a purified (polyclonal) serum and, preferably, to a non-purified crude polyclonal serum. The antibody/serum is obtainable, and preferably obtained, by the method described herein and illustrated in the appended examples or by other methods known in the art.

As exemplified in the experimental part, said anti-RF antibody, in particular said anti-RF1 antibody, may specifically deplete one particular RF (e.g. RF1 (e.g. having the amino acid sequence of SEQ ID NO: 6)) keeping (an-)other RF(s) (e.g. RF2 (e.g. having the amino acid sequence of SEQ ID NO: 44)) active. In this case, a nonsense-codon suppressing agent (e.g. suppressor tRNA) can bind to the corresponding STOP-codon (e.g. UAG) of the first RF (e.g. RF1) and the second RF (e.g. RF2) is still capable to accomplish normal termination at the corresponding second STOP-codon (e.g. UGA). Said first STOP-codon may be an artificial STOP-codon lying inside of the open reading frame of a mRNA to be translated. Said second STOP-codon may lie at the end of said open reading frame.

The term “release factor” as used herein relates to any factor(s) that is/are capable to bind to the A-site of a ribosome programmed by a stop codon, whereby the stop codon is defined as mentioned herein above. By binding to said A-site, said release factor causes termination of the elongation of a peptide-chain during translation process, and thereby leads to a release of the nascent peptide-chain from the ribosomal complex. Preferably, the term “release factor” refers to release factors that are contained in cell-free translation systems. In context of the present invention, the term “release factor” also relates to a fragment of a release factor as defined herein. The term “fragment” (of a release factor) as used herein relates to fragments of a length of at least 30, at least 40, at least 50, more preferably at least 60, ever more preferably at least 65 amino acid residues of a (native) RF as defined herein. The amino acid sequence of RFs are known in the art and also specified herein below. Preferably, said fragment comprises at least such stretch of amino acids that (polyclonal) antibodies may be raised against this fragments and that these obtained antibodies are capable to precipitate and/or crosslink a release factor in a cell-free translation system.

The proteins, polypeptides or peptide to be synthesized in the cell-free translation system as employed herein or in the in vivo expression system as defined herein, may, inter alia, be selected from the group consisting of enzymes, hormones, lectins, metabolic proteins, pheromones, proteins of signal transduction pathways, signal proteins, transporter molecules, proteins involved in translation and/or transcription processes, structural proteins, antibodies, antibody fragments, antibody parts, single-chain antibodies (scFvs), diabodies, markers (marker proteins), reporters (reporter proteins) and the like. Also envisaged are proteinaceous compounds, like toxins, e.g. ricin and the like. Also growth factors and cytokines are envisaged to be expressed, monitored and tracked by the methods provided herein. Also fragments of these proteins may be expressed and “monitored and/or tracked” by the uses and methods provided herein. In context of further embodiments of the invention, in particular the herein disclosed methods for immobilization of proteins/polypeptides/peptides and their corresponding (potential) recovery, other examples of proteins/polypeptides/peptides are provided are provided which may also be monitored/tracked by the method/uses provided herein. The person skilled in the art will readily understand that the uses and methods of the present invention can be widely employed and that the proteins/polypeptides/peptides to be synthesized in the cell-free system or in vivo expression system may be of or may be derived from any organism or may be of complete synthetic or recombinant origin. Accordingly, the present invention is not limited to a specific “X”/“X′” in the fusion construct/fusion protein as defined herein (“X-esterase”/“esterase-X”). Also synthetic and/or non-naturally occurring proteinaceous structures may be expressed, monitored and/or tracked by the means and methods provided herein.

Said protein, polypeptide/polypeptides/peptides to be synthesized is (during its synthesis) covalently bound to an esterase. The construct of the covalently bound protein, polypeptide/polypeptides/peptides and the esterase may be in the form of a fusionprotein, fusionpolypeptide or fusionpeptide. The embodiments described below for the fusion constructs encoded by the inventive vectors apply for this fusionprotein, fusionpolypeptide or fusionpeptide, mutatis mutandis.

Besides the above recited naturally occurring, yet recombinantly produced proteins to be synthesized in the cell-free system or in vivo expression systems in combination with esterase/esterase activity, it is also envisaged that the present uses and methods be employed in the production of alloproteins. Also the synthesis of such alloproteins may be monitored and/or tracked by the methods provided herein.

In context of the present invention, the term “alloproteins” refers to proteins that are achieved by applying the subject-matter of the present invention. Said term also refers to proteins having covalently bound a non-proteinaceous molecule which usually is not part of (the) naturally occurring protein(s). Said alloprotein may, for example, comprise a puromycin and/or derivative thereof as defined herein. Furthermore, said proteinaceous molecule may comprise an unnatural amino acid, e.g. as described in Gilmore (1999), Topics in Current Chemistry, 202, 77-99. Furthermore, said molecule being covalently bound to and comprised in the alloprotein, might be a functional substituent. Various functional substituents of proteins are well-known in the art. For instance, these functional substituents may be oligosaccharides, lipids, fatty acids, phosphates, acetates or other functional groups naturally occurring to modify polypeptide chains of functional proteins. (Eisele (1999), Bioorganic and Medicinal Chemistry 7, 193-224). Furthermore, said molecule might be a residue of a puromycin (-derivative) as defined herein and/or a puromycin (derivative) as defined herein itself and/or the puromycin derivative of the present invention itself.

The alloproteins produced by the method of the present invention, may be used in a wide variety of applications, for example the preparation of synthetic enzymes (Corey (1987), Science, 238, 1401-1403), gene therapy (Zanta (1999), Proc. Natl. Acad. Sci. U.S.A., 96, 91-96), construction of protein microarray (Niemeyer (1994), Nucleic Acid Res., 22, 5530-5539), creation of molecular scale devices (Keren (2002), Science, 297, 72-75), and development of immunological assays (Niemeyer (2003), Nucleic Acids Res., 31, e90).

The method for the production of alloproteins, as described herein, offers the possibility that any desired chemical structure including different dyes, affinity tags, spin labels etc. may be covalently conjugated with proteins at a high yield. This opens the way for variety of applications. For example, puromycin modified with an azide group may be used to covalently attach to the C-terminus of proteins for subsequent one site addressed Staudinger reaction (Kohn (2004), Angew. Chem. Int. Ed Engl., 43, 3106-3116) and utilization of puromycin carrying α-thio-ester group may allow to use the protein ligation technology (Lovrinovic (2003), Chem. Commun. (Camb.), 822-823). This conjugation technology can further be used to develop concepts for preparation of protein arrays and novel tools to study protein interactions (Ramachandran (2004), Science, 305, 86-90).

The alloproteins described herein may as proteinaceous part comprise proteins. These proteins may, inter alia, be selected from the group consisting of enzymes, hormones, pheromones, structural proteins and the like. It is also envisaged that said alloproteins only comprise fragments, like functional, active fragments of said enzymes, hormones, pheromones, structural proteins and the like. Also proteinaceous toxins are envisaged. The person skilled in the art is readily in the position to understand that the embodiments provided herein are easily transferable to other proteins, polypeptides or peptides. The person skilled in the art can, e.g. replace the “GFP”, “eGFP”, “esterase-GFP”, or “esterase-eGFP”, as employed as “detectable marker” in the appended examples by any desired protein, polypeptide or peptide, without deferring from the gist of the present invention. Said proteins may act as core-proteins and/or starting proteins for the alloproteins to be produced in the cell-free translation system or in vivo expression system as employed herein and corresponding additional chemical structures may be added to said proteinaceous part. Accordingly, for example a hormone may be produced which comprises at least, e.g. one additional unnatural amino acid or (e.g.) a puromycin-derivative as defined herein.

Said alloproteins may also be conjugates of proteins and nucleic acids having specific sequences. Said conjugates allow to link the properties of these two distinct groups of biopolymers within one molecule. Therefore, said conjugates can be used in a wide variety of applications, where said linkage of said properties of these two distinct groups of biopolymers is advantageous. These applications are well known in the art and may, for instance, include the preparation of synthetic enzymes (Corey (1987), Science, 238, 1401-1403), gene therapy (Zanta (1999), Proc. Natl. Acad. Sci. U.S.A., 96, 91-96), construction of protein microarray (Niemeyer (1994), Nucleic Acid Res., 22, 5530-5539), creation of molecular scale devices (Keren (2002), Science, 297, 72-75), and development of immunological assays (Niemeyer (2003), Nucleic Acids Res., 31, e90).

As an example also provided in the experimental part of this invention, the alloproteins produced by the method of the present invention, e.g. by using the cell-free translation system or the in vivo systems disclosed herein, comprises the above described esterases/esterase activity as marker or tracking molecule.

The alloprotein may comprise a covalently-bound puromycine or a derivative thereof and/or wherein said protein, polypeptide or peptide to be synthesized comprises an amino acid, delivered by suppressor aminoacyl-tRNA. Preferred, said suppressor aminoacyl-tRNA may be suppressor aminoacyl-tRNA^{Ser(CUA)}.

Preferably, the alloprotein comprises a C-terminal, covalently-bound puromycine or a derivative thereof.

The puromycin derivative which are employed within the present invention may be particularly useful in the use of an esterase, the vectors, the methods and the kit. For example, the puromycin derivative of the present invention may be useful in mRNA display. The yields of the mRNA-protein coupling in mRNA display (Roberts, (1997) JW Proc Natl. Acad. Sci. U.S.A. 94, 122297-302) are usually low. The reason is the low tolerance of the ribosomal A-site for 5′-extended puromycin-nucleic acid conjugates and the high selectivity of this site for EF-Tu.GTP dependent delivery of the aminoacyl-tRNA (Starck (2002), RNA, 8 890-903) RNA molecules that are longer than 5-6 nucleotide residues can not enter the ribosomal A-site in EF-TuGTP independent manner. There is, however, a possibility to circumvent this problem by using the puromycin derivative of the present invention. By the provision of said puromycin derivative, instead of covalent attachment of RNA to the 5′-position of puromycin an alternative strategy by which the RNA (mRNA) or other functional groups are attached directly or via a linker to the nucleobases of puromycine-derived olignucleotides (e.g. CpCpPu or CpPu) can be used. Example for this type of conjugation is provided in the experimental part of this invention

The potential residues of said puromycin derivative have been described above and same applies here for the advantageous puromycin attachment sites. Likewise, the linkers between the puromycin (-derivative) and the residues which may, inter alia be employed have been described above in context of other embodiments.

In the puromycin derivative, the covalently attached residue may be selected from the group consisting of a Cy3-fluorophore, biotin or another affinity tag, a reactive group for affinity labelling or any other reporter group. Further, the residue may be a(n) (other) spectroscopic reporter.

It is evident for the skilled artesian that other residues may be employed in context of this invention.

In a further preferred embodiment of the herein provided use of a puromycin derivative, the linker is an aliphatic amine, in particular an aliphatic amine forming an amide with a fatty acid.

Another preferred cell-free translation system to be employed in the context of this invention is a cell-free translation system, wherein said nonsense-codon suppressing agent is puromycin or a derivative thereof and/or a suppressor tRNA. Said suppressor tRNA may be, e.g. suppressor tRNA^{Ser(CUA)}.

The nonsense-codon suppressing agent may also be e.g. selected from the group consisting of:
(a) Puromycin;
(b) 5′-OH-CpPuromycin;
(c) 5′-OH-CpCpPuromycin;
(d) a puromycin derivative as defined in (a) to (c) having a residue covalently attached directly or via a linker to its 5′-position;
(e) a puromycin derivative as defined in (a) to (d) having a residue covalently attached directly or via a linker to the element N⁴ of the cytosine-residue of an 5′ attached cytidine-residue; and
(f) a puromycin derivative as defined in (a) to (e) having a residue covalently attached directly or via a linker to the element C⁵ of the cytosine-residue of an 5′ attached cytidine-residue;
   whereby a nonsense-codon suppressing agent as defined in (e) is preferred.

For example, the residue to be covalently attached to the puromycin (or a derivate thereof), may be selected from the group consisting of nucleic acids like DNA, RNA, locked DNA, PNA, oligonucleotide-thiophosphates and substituted ribooligonucleotides and other nucleic acids. It is also envisaged that other residues, like peptides or “tags” can be attached to said puromycin to be integrated in a protein during its in vitro synthesis.

In further examples, the residue, covalently attached to said puromycin or said derivative thereof, may be selected from the group consisting of a Cy3-fluorosphore, biotin or an other affinity tag, a reactive group for affinity labelling or any other reporter group (for review see Gilmore (1999), Topics in Current Chemistry, 202, 77-99). The reactive group, for instance, can be an a-zide group to use for subsequent one site addressed Staudinger reaction (Kohn (2004), Angew. Chem. Int. Ed Engl., 43, 3106-3116) or an α-thio-ester group to use for the protein ligation technology (Lovrinovic (2003), Chem. Commun. (Camb.), 822-823). In further examples, the residue, covalently attached to said puromycin or said derivative thereof, may be also be a(n) (other) spectroscopic reporter.

In the context of the present invention, the term “linker” refers to a molecule capable to connect said puromycin (-derivative) and said residue covalently.

For example, the linker between the puromycin (-derivative) and said residue may be an aliphatic amine derivative, preferably forming an amide with a fatty acid attached to a polyoxyamine. Preferably, said linker may comprise the following molecule:

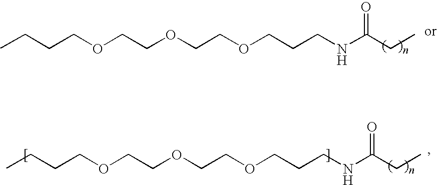

wherein the part of the molecule indicated in squared brackets may be of different length, e.g. may be elongated by additional or shortened by less carbon residues and/or oxygen residues.

Preferably, in said linker, (n) is at least 3, preferably at least 5 carbon residues. Yet, the amount of carbon residues (n) may most preferably be 5 or 9.

Further, the linker between the puromycin (-derivative) and said residue may act as a “place holder” that warrants the undisturbed entrance of the puromycin (-derivative) into the A-site of the ribosome.

“Nonsense-codon suppressing agent” are known in the art, as documented above. However, the “nonsense-codon suppressing agent” comprised in a cell-free translation system of the present invention or as described herein may also be selected from the group consisting of:
(a) 5′-OH-GpCpPuromycin;
(b) 5′-OH-GpCpCpPuromycin;
(c) 5′-OH-GpApCpCpPuromycin;
(d) 5′-OH-GpCpApCpCpPuromycin;
(e) 5′-OH-GpCpCpApCpCpPuromycin;

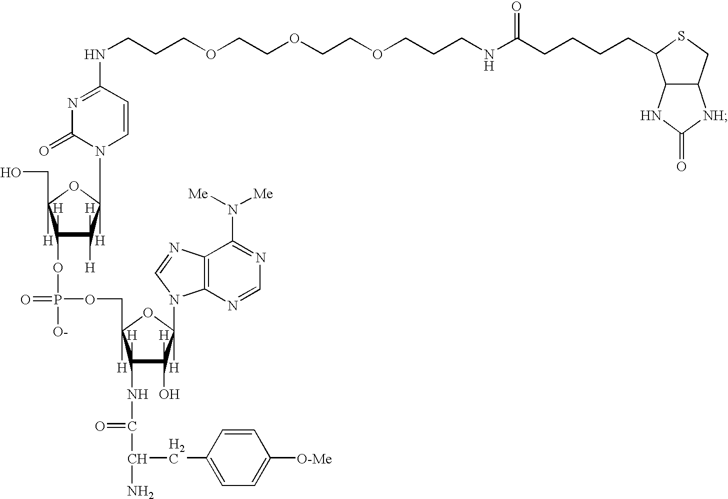

(g)

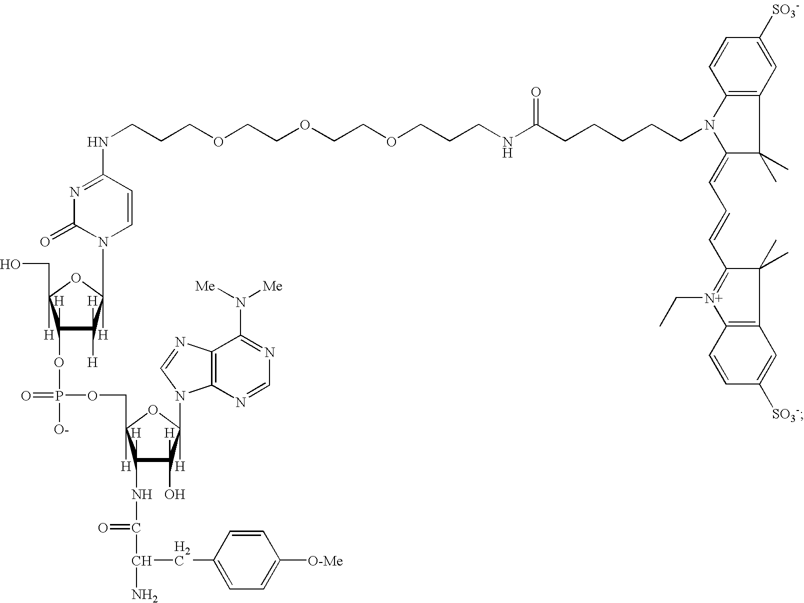

(h)

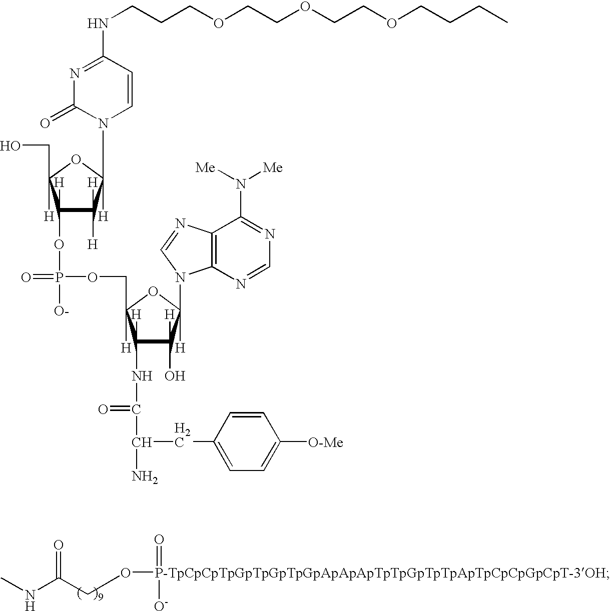

and
(i)

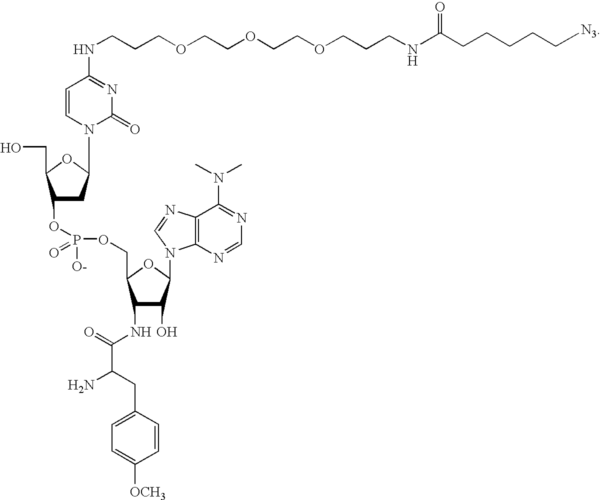

As already pointed out above, the cell-free translation system described herein and, inter alia, to be employed in the context of this invention may comprise a component which is capable of inhibiting and/or negatively interfering with a release factor comprised in said cell-free translation system. In context of this invention, it was found that an antibody directed against such (a) release factor(s) is particularly useful in inhibiting the function of such a factor. Such an anti-release factor antibody is to be used which is capable of specifically inactivating said release factor, e.g. by precipitation and/or crosslinking, whereas or other components remain intact, is of prokaryotic or eukaryotic origin, more preferable it is of prokaryotic origin, even more preferably it is from E. coli.

For example, a release factor to be inactivated from E. coli may be the release factor 1, the release factor 2, the release factor homolog 1, the release factor homolog 2, the release factor homolog 3 or the release factor homolog 4. Said release factors may be encoded by the nucleotide sequences as shown in SEQ ID NOs: 5, 43, 45, 47 or 49 and/or may have the amino acid sequences as shown in SEQ ID NOs: 6, 44, 46, 48, 50 or 51. Most preferred, and also shown in the experimental part, said release factor contained in said cell-free translation system and to be inactivated is the release factor 1 from E. coli. Said most preferred release factor may be encoded by the nucleotide sequence as shown in SEQ ID NO: 5 and/or may have the amino acid sequence as shown in SEQ ID NO: 6.

The release factor contained and to be inactivated in the cell-free translation system of the present invention may be different from the release factor, against which the antibody to be employed was directed and/or generated. For example, the release factor contained and to be inactivated in the cell-free translation system of the present invention may be from E. coli. Accordingly, sad translation system comprises RF1 from E. coli. Yet, as shown in the examples, the anti-release factor antibody, precipitating and/or crosslinking said release factor, was generated against a release factor from Thermus thermophilus, namely against RF1 from Thermus thermophilus. Said RF1 from Thermus thermophilus may be encoded by the nucleotide sequence as shown in SEQ ID NO: 3 and/or may have the amino acid sequence as shown in SEQ ID NO: 4.).

In an eukaryotic context, the release factor to be inactivated by a specific crosslinking and/or precipitating antibody may be from rabbit, fruit fly or yeast. Preferably, said release factor to be inactivated by antibodies is a rabbit RF. For instance, said release factor is the release factor 1 or the release factor 3 from rabbit, release factor 1 from fruit fly or the release factor 1 or the peptide chain release factor 1 from yeast. Said exemplified release factors may be encoded by the nucleotide sequences as shown in SEQ ID NOs: 52, 54, 56 or 58, respectively, and/or may have the corresponding amino acid sequences as shown in SEQ ID NOs: 53, 55, 57 or 59. Corresponding antibodies may be prepared by methods known in the art, for example by the generation of a polyclonal serum against said release factors. “inactivating antibodies to be employed in the cell-free translation system of the present invention are, as described herein, antibodies and/or antibody molecules which are capable of precipitating and or crosslinking the release factor(s) comprised in the cell-free translation system of the present invention. Said “inactivation” may be a complete or a partial inactivation. As pointed out above, said “inactivation” leads to an inactivation of the function of said release-factors of at least 60%, more preferably of at least 70%, more preferably of at least 80% and more preferably of at least 90%. The corresponding inactivation of the release-factors by the addition of the precipitating and/or crosslinking antibodies and/or antibody molecules can be measured by methods known in the art. For example, the precipitating and/or deactivating activity of anti-RF polyclonal antibodies can be measured by the residual RF activity in the in vitro translation system, by testing the hydrolysis of a peptide from peptidyl-tRNA located in the P-site (Freistroffer Proc Natl Acad Sci USA. (2000) 97, 2046-51. or by a gel electrophoresis followed by Western blotting, which is being a common laboratory praxis.

In particular, the release factor contained in said cell-free translation system and, potentially, to be inactivated may be selected from the group consisting of:
(a) a release factor encoded by a nucleotide sequence comprising a nucleotide sequence as shown in any one of SEQ ID NOS: 3, 5, 5, 43, 45, 47, 49, 52, 54, 56, 58 and 60;
(b) a release factor encoded by a nucleotide sequence coding for a polypeptide comprising an amino acid sequence as shown in any one of SEQ ID NOS: 4, 6, 44, 46, 48, 50, 51, 53, 55, 57, 59 and 61;
(c) a release factor which is encoded by a nucleotide sequence of a nucleic acid molecule that hybridizes to the complement strand of a nucleic acid molecule comprising a nucleotide sequence as defined in (a) or (b) and which releases a translation product from a ribosome in a cell-free translation system;
(d) a release factor which comprises an amino acid sequence as shown in any one of SEQ ID NOS: 4, 6, 44, 46, 48, 50, 51, 53, 55, 57, 59 and 61;
(e) a release factor which comprises an amino acid sequence which is at least 40% identical to the full length amino acid sequence as shown in any one of SEQ ID NOS: 4, 6, 44, 46, 48, 50, 51, 53, 55, 57, 59 and 61; and
(f) a release factor encoded by a nucleotide sequence which is degenerated to a nucleotide sequence as defined in any one of (a) to (c).

It is immediately evident form the above that the inhibition of the release factor is only and merely one embodiment of the uses and methods of the present invention. The use of esterase/esterase activity as disclosed herein is in no means limiting to the herein also described preparation and synthesis of alloproteins, synthesis of which is to be monitored and/or tracked in accordance with this invention. Yet, as also detailed below, the synthesis of alloproteins in combination with esterase is particularly useful in the preparation of alloprotein-esterase fusion constructs. As shown below, the use of esterase in this context also provides for means and methods to immobilize (allo-) proteins, inter alia on solid surfaces. Further embodiments are provided below.

As detailed above, the invention is based on the advantageous use of esterase/esterase activity in the (in vitro) synthesis of proteins, also alloproteins. In a most preferred embodiment, said proteins/alloproteins and the like are produced in the format of an esterase-fusion construct, preferably in the format “X-esterase” or “esterase-X”, i.e. the desired target protein, polypeptide or peptide being located N- or C-terminally of the esterase activity/esterase function bearing moiety. It is within the skills of the artesian to deliberate the expressed target protein, polypeptide or peptide from the esterase moiety after expression. For example, the fusion construct to be expressed, monitored and/or tracked in accordance with the means and methods of the invention may additionally comprise a (proteolytically) cleavable tag which may be used to separate the desired “X” or “X′” from the esterase moiety. Corresponding examples are provided herein and are illustrated in the appended examples. Accordingly, known cleavage methods may be employed, like chemical or enzymatic methods. It is also envisaged that the expression product “X-esterase”/“esterase-X” comprises known cleavage sites (cleavage tags), sites between the “X” and the esterase moiety. Such cleavage sites are, inter alia, disclosed in Stevens (2003, Drug Discovery World, 4, 35-48) and LaVallie (1994, Enzymatic and chemical cleavage of fusion proteins. In Current Protocols in Molecular Biology. pp. 16.4.5-16.4.17, John Wiley and Sons, Inc, New York, N.Y.); and comprise, but are not limited to, hydroxylamine cleavage (cleavage between Asn-Gly), enterokinase cleavage (cleavage after Asp-Asp-Asp-Asp-Lys), Factor Xa protease cleavage (cleavage after Ile-Glu/Asp-Gly-Arg) and the like. In context of the present invention, factor Xa protease cleavage is preferred, but also other cleavages are envisaged.

The invention also provides for a vectors, whereby said vectors are characterized in comprising a nucleic acid molecule coding for an esterase and expressing an esterase fusionprotein. Preferably, said vector comprises a nucleic acid molecule coding for an esterase and comprising in frame at least one multiple cloning site for a part X of an esterase-X fusionprotein, whereby the fusionprotein to be encoded may be of the format “X-esterase” or “esterase-X”. Corresponding examples are given below and in the append examples. Vectors useful in particular in cell-free systems are known in the art and comprise but are not limited to plasmids, cosmids as well as viral vectors and bacteriophages or another vector used e.g. conventionally in genetic engineering. Said vectors may, besides the esterase activity and the protein/polypeptide “X” desired to be expressed comprise further genes such as marker genes which allow for the further selection The vector of the invention is, accordingly, an expression vector, in which the nucleic acid molecule coding for an esterase/esterase activity is operatively linked to expression control sequence(s) allowing expression in prokaryotic or eukaryotic cell-free systems as described herein. The term “operatively linked”, as used in this context, refers to a linkage between one or more expression control sequences and the coding region in the polynucleotide to be expressed (preferably “X-esterase” and “esterase-X”) in such a way that expression is achieved under conditions compatible with the expression control sequence.

A polynucleotide as defined above, coding for the fusion protein (“X-esterase”/“esterase-X”) defined herein, whereby said polynucleotide is fused to a heterologous polynucleotide (“X”), preferably encoding a heterologous polypeptide (“X”) is to be employed in context of this invention. This heterologous polypeptide may, inter alia, be a marker, like a green fluorescent protein or HA, as shown in the appended examples. However, every desired protein to be expressed may be employed as “X” in the herein defined fusion construct “X-esterase”/“esterase-X”. Preferably, said nucleic acid molecule(s)/polynucleotide(s) of the present invention is part of a vector. Said vector is preferably a gene expression vector. Therefore, the present invention relates in another embodiment to a vector comprising the nucleic acid molecule coding for an esterase fusion construct as disclosed herein. Said vector is capable of expression. Such a vector may be, e.g., a plasmid, cosmid, virus, bacteriophage or another vector used e.g. conventionally in genetic engineering, and may comprise further genes such as marker genes which allow for the selection of said vector in a suitable host cell and under suitable conditions or in suitable expression/translation systems.

The nucleic acid molecules coding for such esterase-fusion constructs may be inserted into expression vectors, like several commercially available vectors. Nonlimiting examples include plasmid vectors compatible with mammalian cells, such as pGEM-T (Promega), pIVEX 2.3d (Roche Diagnostics), pUC, pBluescript (Stratagene), pET (Novagen), pREP (Invitrogen), pCRTopo (Invitrogen), pcDNA3 (Invitrogen), pCEP4 (Invitrogen), pMC1 neo (Stratagene), pXT1 (Stratagene), pSG5 (Stratagene), EBO-pSV2neo, pBPV-1, pdBPVMMTneo, pRSVgpt, pRSVneo, pSV2-dhfr, pUCTag, pIZD35, pLXIN and pSIR (Clontech) and pIRES-EGFP (Clontech). The present invention provides also for a specific vector, denoted pEst2, as also shown in SEQ ID No. 9. Further nonlimiting examples include baculovirus vectors (in particular useful in in vitro translation systems or in vivo expression systems based on insect cells) such as pBlueBac, BacPacz Baculovirus Expression System (CLONTECH), and MaxBac™ Baculovirus Expression System, insect cells and protocols (Invitrogen), which are available commercially and may also be used to produce high yields of biologically active protein. (see also, Miller (1993), Curr. Op. Genet. Dev., 3, 9; O'Reilly, Baculovirus Expression Vectors: A Laboratory Manual, p. 127). In addition, prokaryotic vectors such as pcDNA2 and yeast vectors such as pYes2 are nonlimiting examples of other vectors suitable for use with the present invention. For vector modification techniques, see Sambrook and Russel (2001), loc. cit. Vectors can contain one or more replication and inheritance systems for cloning or expression, one or more markers for selection in the host, e.g., antibiotic resistance, and one or more expression cassettes. The coding sequences inserted in the vector can be synthesized by standard methods, isolated from natural sources, or prepared as hybrids. Ligation of the coding sequences to transcriptional regulatory elements (e.g., promoters, enhancers, and/or insulators) and/or to other amino acid encoding sequences can be carried out using established methods.

The vectors of the present invention are characterised in that they carry a nucleic acid molecule encoding an esterase (esterase-activity) or a functional fragment thereof and that said vectors also comprise an additional cloning site, mostly an multiple cloning site, wherein a nucleic acid molecule coding for the desired protein, polypeptide or peptide (to be expressed in a given cell-free translation system) be introduced. Said introduction is desired to lead to a nucleic acid molecule, comprised in said vector, which is capable of expressing the protein, polypeptide or peptide desired in form of a fusionprotein, fusionpolypeptide or fusionpeptide, wherein the further part of said fusion molecule is the esterase (esterase activity) or a functional fragment thereof. Accordingly, the vector of the present invention, is a vector to be employed in the in vitro synthesis (in cell-free translation systems) of fusionproteins, fusionpolypeptides or fusionpeptides in the format “X-esterase” or “esterase-X”. The embodiments described above for such fusion constructs apply here, mutatis mutandis. Accordingly, said inventive vector is also designed to lead to the expression of a fusion-construct “X-esterase” or “esterase-X”, whereby said “X” being the desired protein, polypeptide or peptide to be expressed in the system and whereby “esterase” denotes the esterase/esterase-activity used for monitor and/or track the efficacy of the translation system, as detailed above.

The vectors of the present invention are characterised in that they carry a nucleic acid molecule encoding an esterase (esterase-activity) or a functional fragment thereof and that said vectors also comprise an additional cloning site, mostly an multiple cloning site, wherein a nucleic acid molecule coding for the desired protein, polypeptide or peptide (to be expressed in a given cell-free translation system) be introduced. Said introduction is desired to lead to a nucleic acid molecule, comprised in said vector, which is capable of expressing the protein, polypeptide or peptide desired in form of a fusionprotein, fusionpolypeptide or fusionpeptide, wherein the further part of said fusion molecule is the esterase (esterase activity) or a functional fragment thereof. Accordingly, the vector of the present invention, is a vector to be employed in the in vitro synthesis (in cell-free translation systems) of fusionproteins, fusionpolypeptides or fusionpeptide in the format “X-esterase” or “esterase-X”. The embodiments described above for such fusion constructs apply here, mutatis mutandis. Accordingly, said inventive vector is also designed to lead to the expression of a fusion-construct “X-esterase” or “esterase-X”, whereby said “X” being the desired protein, polypeptide or peptide to be expressed in the system and whereby “esterase” denotes the esterase/esterase-activity used for monitor and/or track the efficacy of the translation system, as detailed above.

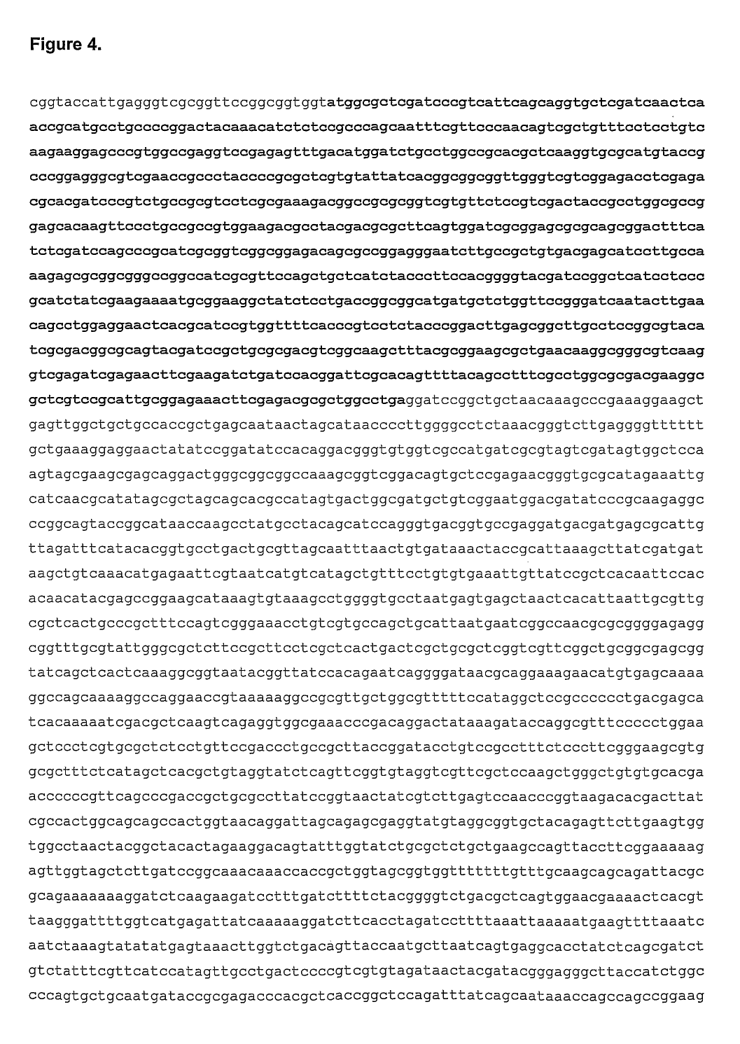

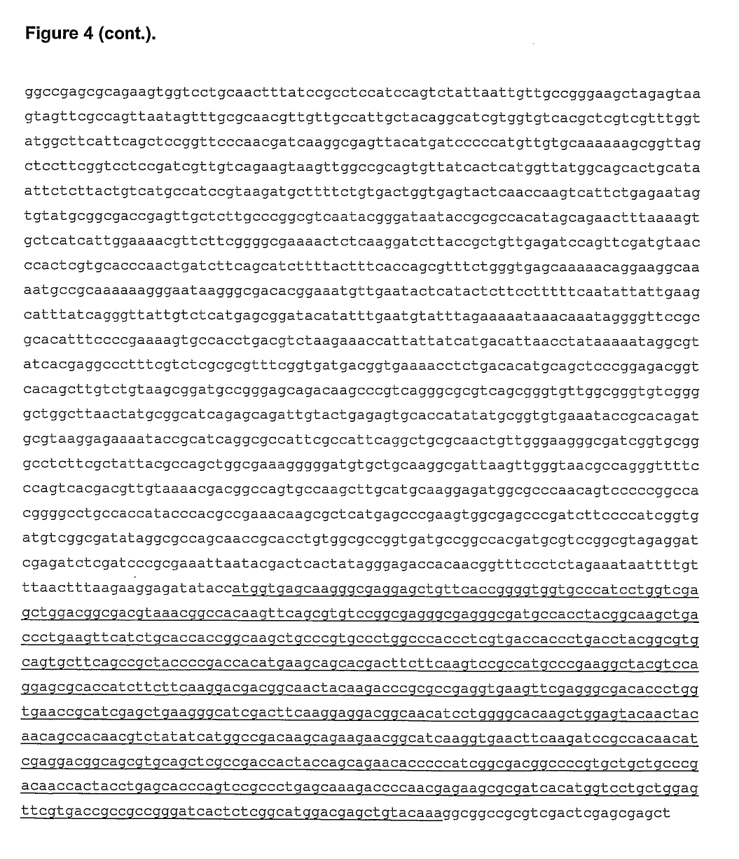

Accordingly, it is also desired and possible that the inventive vectors, comprising a nucleic acid molecule encoding said esterase and/or esterase-activity (or a functional fragment thereof) express molecules, whereby at least one further, additional peptide, polypeptide or peptide is expressed. Here it is referred to the embodiment above, relating to “X” and “X′”. The components “X”, “X′” etc. to be expressed (in frame) with at least one esterase and/or esterase activity may, like in the construct “X-esterase”/“esterase-X”, be separated by a linker/linker structure. Such a construct may, e.g. be characterized as “X′-X-esterase”, “X-X′-esterase”, “esterase-X′-X” or “esterase-X-X′”. Most preferably, said linker/linker structure is a cleavable linker, e.g. a linker which may be, e.g. chemically or enzymatically cleaved. Further embodiments described above for such linkers, apply here, mutatis mutandis. Vectors of the invention are also illustrated herein and in the appended examples. One example of such a vector is given in SEQ ID NO: 8, and FIG. 4. Namely, said vector comprises a nucleic acid molecule coding for Est2 and said “X” (or said “X′”) is eGFP. It is immediately evident for a person skilled in the art that the nucleic acid molecule coding eGFP can easily replaced (by recombinant means) with another nucleic acid molecule coding for the protein, polypeptide or peptide desired to be expressed in a given cell-free translation system.

Furthermore, the vectors may, in addition to the nucleic acid sequences of the invention, i.e. a a nucleic acid molecule coding for esterase-fusion constructs as provided herein, comprise expression control elements, allowing proper expression of the coding regions in suitable hosts or in in vitro translation systems. Such control elements are known to the artisan and may include a promoter, translation initiation codon, translation and insertion site or internal ribosomal entry sites (IRES) (Owens, Proc. Natl. Acad. Sci. USA 98 (2001), 1471-1476) for introducing an insert into the vector. Preferably, the nucleic acid molecule of the invention is operatively linked to said expression control sequences allowing expression in eukaryotic or prokaryotic cells or in in vitro translation systems.

Control elements ensuring expression in eukaryotic and prokaryotic cells are well known to those skilled in the art. As mentioned above, they usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in for example mammalian host cells comprise the CMV-HSV thymidine kinase promoter, SV40, RSV-promoter (Rous sarcome virus), human elongation factor 1α-promoter, CMV enhancer, CaM-kinase promoter or SV40-enhancer.

For the expression in prokaryotic cells, a multitude of promoters including, for example, the tac-lac-promoter, the lacUV5 or the trp promoter, has been described. Beside elements which are responsible for the initiation of transcription such regulatory elements may also comprise transcription termination signals, such as SV40-poly-A site or the tk-poly-A site, downstream of the polynucleotide. In this context, suitable expression vectors are known in the art such as Okayama-Berg cDNA expression vector pcDV1 (Pharmacia), pRc/CMV, pcDNA1, pcDNA3 (In-Vitrogene), pSPORT1 (GIBCO BRL) or pGEM-T (Promega), or prokaryotic expression vectors, such as lambda gt11. In the appended examples, pGEM-T (Promega) and pIVEX2.3d (Roche Diagnostics, Mannheim, Germany) were employed. It is in the routine working skills of the artesian to adapt and generate desired gene expression vectors commercially available vectors.

An expression vector according to this invention is at least capable of directing the expression of the fusion nucleic acids and fusion proteins described herein. Suitable origins of replication include, for example, the Col E1, the SV40 viral and the M 13 origins of replication. Suitable promoters include, for example, the T7 promoter (as employed in the appended examples), the cytomegalovirus (CMV) promoter, the lacZ promoter, the gal10 promoter and the Autographa californica multiple nuclear polyhedrosis virus (AcMNPV) polyhedral promoter. Suitable termination sequences include, for example, the T7 terminator, the bovine growth hormone, SV40 terminator, lacZ terminator and AcMNPV terminator polyhedral polyadenylation signals. Examples of selectable markers include neomycin, ampicillin, and hygromycin resistance and the like. Specifically-designed vectors allow the shuttling of DNA between different host cells, such as bacteria-yeast, or bacteria-animal cells, or bacteria-fungal cells, or bacteria-invertebrate cells.

The vectors provided herein are particular useful in the preparation of specific kits, preferably kits for cell-free translation systems. Such a kit may, inter alia, comprise the cell-free translation system and a further component, comprising a vector of the present invention in which (in frame) the nucleic acid molecule coding for the desired protein, polypeptide or peptide may be ligated. To ligate said nucleic acid molecule “in frame” means, that the nucleic acid molecule coding for least the esterase (esterase function) or a fragment thereof as well as the nucleic acid molecule coding for the desired protein, polypeptide or peptide is transcribed in a manner that the resulting (mRNA) represents the open reading frames for both, said esterase (esterase function) or a fragment thereof and said desired protein, polypeptide or peptide. Therefore, the “in frame” means, that the resulting product of the expression vector is a fusionprotein, fusionpolypeptide or fusionpeptide, comprising at least the esterase (esterase function) or a fragment thereof and the desired protein, polypeptide or peptide or a fragment thereof, wherein the esterase (esterase function) or a fragment thereof as well as the desired protein, polypeptide or peptide is functional. The embodiments provided herein for the inventive vector also apply to nucleic acid molecules comprised in said vectors.

The present invention in addition relates to a host transformed with a vector of the present invention or to a host comprising the nucleic acid molecule and coding for a fusion protein as defined herein (“X-esterase”/“esterase-X”) of the invention. Said host may be produced by introducing said vector or nucleotide sequence into a host cell which upon its presence in the cell mediates the expression of a protein encoded by the nucleotide sequence of the invention or comprising a nucleotide sequence or a vector according to the invention wherein the nucleotide sequence and/or the encoded polypeptide is foreign to the host cell.

By “foreign” it is meant that the nucleotide sequence and/or the encoded polypeptide is either heterologous with respect to the host, this means derived from a cell or organism with a different genomic background, or is homologous with respect to the host but located in a different genomic environment than the naturally occurring counterpart of said nucleotide sequence. This means that, if the nucleotide sequence is homologous with respect to the host, it is not located in its natural location in the genome of said host, in particular it is surrounded by different genes. In this case the nucleotide sequence may be either under the control of its own promoter or under the control of a heterologous promoter. The location of the introduced nucleic acid molecule or the vector can be determined by the skilled person by using methods well-known to the person skilled in the art, e.g., Southern Blotting. The vector or nucleotide sequence according to the invention which is present in the host may either be integrated into the genome of the host or it may be maintained in some form extrachromosomally. In this respect, it is also to be understood that the nucleotide sequence of the invention can be used to restore or create a mutant gene via homologous recombination.

Said host may be any prokaryotic or eukaryotic cell. Suitable prokaryotic/bacterial cells are those generally used for cloning like E. coli, Salmonella typhimurium, Serratia marcescens or Bacillus subtilis. Said eukaryotic host may be a mammalian cell, an amphibian cell, a fish cell, an insect cell, a fungal cell or a plant cell. Said prokaryotic cell may be bacterial cell (e.g., E coli strains BL21 (DE3), HB101, DH5a, XL1 Blue, Y1090 and JM101). Eukaryotic recombinant host cells are also useful. Examples of eukaryotic host cells include, but are not limited to, yeast, e.g., Saccharomyces cerevisiae, Schizosaccharomyces pombe, Kluyveromyces lactis or Pichia pastoris cells, cell lines of human, bovine, porcine, monkey, and rodent origin, as well as insect cells, including but not limited to, Spodoptera frugiperda insect cells and Drosophila-derived insect cells. Also fish and amphibian cells, like Xenopus cells or zebra fish cells (including eggs of amphibians and fishes), may be employed. Mammalian species-derived cell lines suitable for use and commercially available include, but are not limited to, L cells, CV-1 cells, COS-1 cells (like ATCC CRL 1650), COS-7 cells (like ATCC CRL 1651), HeLa cells (like ATCC CCL 2), C1271 (like ATCC CRL 1616), BS-C-1 (like ATCC CCL 26), CHO cells (like ATCC CRL 1859, ATCC CRL 1866) and MRC-5 (like ATCC CCL 171). Also HEK293 cells may be employed and are useful as host cells in accordance with this invention.

The invention also provides for the use of a vector as described herein or of a nucleic acid molecule comprised in said vector (and comprises a coding sequence for a fusion protein as defined herein and comprising an esterase/esterase activity (or a functional fragment thereof)) for monitoring and/or tracking the synthesis of said protein, polypeptide or peptide in a cell free translation system or in an in vivo expression system. As detailed herein and in the appended examples, said monitoring and/or tracking comprises the detection of the function of said esterase/esterase activity.

Furthermore, the invention also provides for a protein, polypeptide or peptide encoded by a vector of the invention. The embodiments described above for the fusion constructs encoded by the inventive vectors apply for the inventive protein, polypeptide or peptide, mutatis mutandis.

Additionally, the invention provides for a kit comprising a vector or a nucleic acid molecule defined herein above, said vector/nucleic acid molecule comprising a coding sequence for an esterase/esterase activity. Said kit is particularly useful in combination with cell-free translation systems and may be part of kit comprising ingredients of such cell-free translation systems. Examples, which are non-limiting, of such ingredients of a cell-free translation system have been given herein above and are also shown in the experimental part. Furthermore, the kit of the invention may also comprise additional components, like e.g. the puromycin-derivatives described above.

Advantageously, the kit of the present invention further comprises, optionally (a) reaction buffer(s), storage solutions and/or remaining reagents or materials required for the conduct of scientific, diagnostic assays and in particular (in vitro) protein-biosynthesis assays. Furthermore, parts of the kit of the invention can be packaged individually in vials or bottles or in combination in containers or multicontainer units.

The kit of the present invention may be advantageously used, inter alia, for carrying out the methods for monitoring and/or teaching protein-/peptide- or polypeptide biosynthesis as described herein and/or it could be employed in a variety of applications referred herein, e.g., as diagnostic kits or as research tools. The kit is also useful in pharmaceutical research. Additionally, the kit of the invention may contain means for detection suitable for scientific, medical and/or diagnostic purposes. Said suitable means for detection comprise, but are not limited to specific esterase substrates. Corresponding examples are given above and are also illustrated in the appended examples. The manufacture of the kits follows preferably standard procedures which are known to the person skilled in the art.

The kit of the invention is particularly useful for monitoring and/or tracking the synthesis of a protein, polypeptide or peptide in a cell-free translation system, wherein said monitoring and/or tracking comprises the detection of the function of said esterase. Said detection may comprise the use of the above recited substrates, which may also be comprised in the kit of the invention. Again, esterase/esterase activities to be detected have been described above and the corresponding embodiments apply here, mutatis mutantis.

As pointed out above, the kit of the invention may be used in combination with a cell-free-translation system or may be part of a kit comprising such a cell-free translation system. Corresponding cell-free systems have been described above.

In a further embodiment, the present invention provides for a method for monitoring and/or tracking the synthesis of a protein, polypeptide or peptide in a cell-free translation system, comprising the step of detecting the function of an esterase. Said function of an esterase/esterase activity can be measured as discussed herein above, e.g. with the use of specific substrates. Again, corresponding embodiments are provided herein and in the appended examples.

As discussed above, the present invention is also useful in tagging proteins with esterase/an esterase activity. Correspondingly, such a tagged protein/polypeptide/peptide, comprising an esterase/esterase activity may be isolated, e.g. from the cell-free translation system with the help of specific esterase-interaction partners. Such an interaction partner/binding partner may, inter alia, be an antibody/antibody molecule or fragment thereof, specifically interacting with/binding to said esterase. In general, as employed herein, the term “antibody” may comprise purified serum, i.e. a purified polyclonal serum. The antibody molecule may also be a full immunoglobulin, like an IgG, IgA, IgM, IgD, IgE, IgY (for example in yolk derived antibodies). The term “antibody” as used in this context of this invention also relates to a mixture of individual immunoglobulins. Furthermore, it is envisaged that the antibody/antibody molecule is a fragment of an antibody, like an F(ab), F(abc), Fv Fab′ or F(ab)₂. Furthermore, the term “antibody” as employed in the invention also relates to derivatives of the antibodies which display the same specificity as the described antibodies. Such derivatives may, inter alia, comprise chimeric antibodies or single-chain constructs.

However, as illustrated herein, also an inhibitor of esterase may function as a corresponding interaction partner.

Accordingly, the present invention also provides for a method for immobilising a protein, polypeptide or peptide comprising the steps of (a) tagging said protein, polypeptide or peptide with an esterase and (b) binding said esterase to an esterase binding molecule (like an antibody) or esterase inhibitor, wherein said esterase binding molecule/inhibitor is immobilized on a solid substrate.

The method for immobilising a protein, polypeptide or peptide may comprise further steps, like (c) cleaving said protein, polypeptide or peptide from said esterase and (d) recovering a purified fraction of said protein, polypeptide or peptide. By steps (c) and (d) the immobilized a protein, polypeptide or peptide may be obtained and/or purified from, inter alia, cell-free translation systems.

The invention also provides for a method for the purification of a protein, polypeptide or peptide, said basically combining the steps provided herein above, namely, comprising the steps of:
(a) expressing in vitro said protein, polypeptide or peptide in a format of an esterase fusion construct or tagging said protein, polypeptide or peptide with an esterase;
(b) immobilizing said esterase fusion construct or said esterase tag protein, polypeptide or peptide according method provided above, e.g. via an specific esterase binding molecule, for example a (specific) anti-esterase antibody or an esterase inhibitor specifically interacting with esterase;
(c) cleaving said protein, polypeptide or peptide from said esterase; and
(d) recovering a purified fraction of said protein, polypeptide or peptide.

As is evident from the embodiments provided herein said tagging of the protein, polypeptide or peptide to be immobilized, purified and/or obtained with said esterase is effected through the production of a fusionprotein as defined above as “X-esterase” or “esterase-X”. Said production/expression may take place in a system as defined above, and is, preferably in a cell-free system.

Whereas binding partners, like antibodies, to esterases may be employed to bind said esterase, also inhibitors may be used. A preferred esterase inhibitor in this context is a trifluoromethyl ketone.

The cleavage of the polypeptide (see step (c) of the herein described method for immobilising a protein, polypeptide or peptide) may be effected by, inter alia, enzymatic cleavage, in particular by cleavage of a linker structure comprised between the esterase and a protein/polypeptide/peptide immobilized by the method provided above. Such linker structure has been discussed herein above in context of the ester-fusionproteins/fusionconstructs to be synthesized in the context of the herein described in vitro biosynthesis.

Most preferably, said cleavage is affected by an enzyme like a protease. A particular preferred protease in this context is factor XA protease.

It is also envisaged that desired protein/polypeptide/peptide immobilized by the method provided herein are cleaved from the esterase, even if there is no linker present between esterase/esterase activity and the desired protein/polypeptide/peptide.

As pointed out above, the cleavage of the desired protein/polypeptide/peptide is merely one embodiment of the methods for immobilization as described above and the appended examples (illustratively example 7). It is, however, also envisaged that the immobilized protein/polypeptide/peptide is not cleaved from the esterase part. This embodiment is particularly useful in the generation of protein-/polypeptide-/peptide-coated matrices, like slides, chips and the like. Accordingly, the present invention also provides for a method of immobilization of protein/polypeptides/peptides (via esterase) to supports, preferably solid supports. Such solid supports may comprise, but are not limited to glass, cellulose, polyacrylamide, nylon, polycabonate, polystyrene, polyvinyl chloride or polypropylene or the like. Accordingly, these supports are well known in the art and also comprise, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc. The antibodies of the present invention may be bound to many different carriers. Examples of well-known carriers include glass, polystyrene, polyvinyl chloride, polypropylene, polyethylene, polycarbonate, dextran, nylon, amyloses, natural and modified celluloses, polyacrylamides, agaroses, and magnetite. The nature of the carrier can be either soluble or insoluble for the purposes of the invention.

It is, as already mentioned above, also envisaged that the present invention be useful in the preparation of “arrays”, in a particular of microarrays. Here not only naturally occurring (and recombinantly expressed) proteins/polypeptides/peptides may be employed, but also the use of the above described alloproteins is envisaged. The proteins/polypeptides/peptides as well as the alloproteins immobilized by the method provided herein, may accordingly be used in a wide variety of applications, for example the preparation of synthetic enzymes (Corey (1987) loc. cit), gene therapy (Zanta (1999) loc. cit), construction of protein microarrays (Niemeyer (1994), loc. cit), creation of molecular scale devices (Keren (2002), loc. cit), or the development of immunological assays (Niemeyer (2003), loc. Cit). However, these uses are in no means limiting.

Proteins/polypeptides/peptides immobilized to said (solid) support via esterase/esterase activity have been described above and comprise, inter alia, enzymes, hormones, pheromones, signal proteins, structural proteins, markers, reporters and the like. As is evident for the person skilled in the art said proteinaceous molecules to be synthesized in accordance with this invention (preferably in form of a fusionprotein with esterase/esterase activity) may also comprise toxins, for example toxins in pharmaceutical use. Also envisaged are proteins, like cytokines and/or growth factors. These proteins/polypeptides/peptides may, accordingly also be synthesized, monitored, tracked and/or immobilized in accordance with the uses and methods of this invention. Such proteins may include, for example, a toxin such as abrin, ricin A, pseudomonas exotoxin, or diphtheria toxin; a cytokine or growth factor such as tumor necrosis factor, a-interferon, β-interferon, nerve growth factor, platelet derived growth factor, tissue plasminogen activator.

These proteins, e.g. enzymes, toxins, hormones, growth factors and the like may be immobilized by the method provided herein. These immobilized proteins/polypeptides/peptides can, for example, be used in treatment of body fluids, like blood. It is, e.g., envisaged that the proteins are immobilized by the method provided herein and that the body fluids are than brought in contact with the (solid) support comprising the immobilized proteins/polypeptides/peptides. This embodiment is particularly useful in ex corpo-therapies, where, e.g., isolated blood is brought in contact with a biologically active sample. The isolated blood may than be reintroduced in a patient in need of such a treatment.

These and other embodiments are disclosed and encompassed by the description and examples of the present invention. Further literature concerning any one of the compounds, kits, methods and uses to be employed in accordance with the present invention may be retrieved from public libraries, using for example electronic devices. For example the public database “Medline” may be utilized which is available on the Internet, for example under http://www.ncbi.nlm.nih.gov/PubMed/medline.html. Further databases and addresses, such as http://www.ncbi.nim.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The present invention is further described by reference to the following non-limiting figures and examples.

The Figures show:

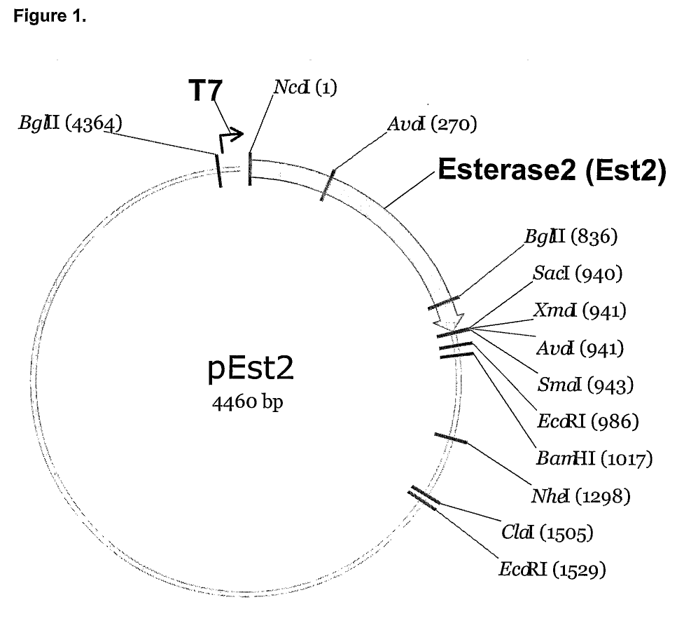

FIG. 1.

Plasmid map of pIVEX 2.3d-Est2 (pEst2).

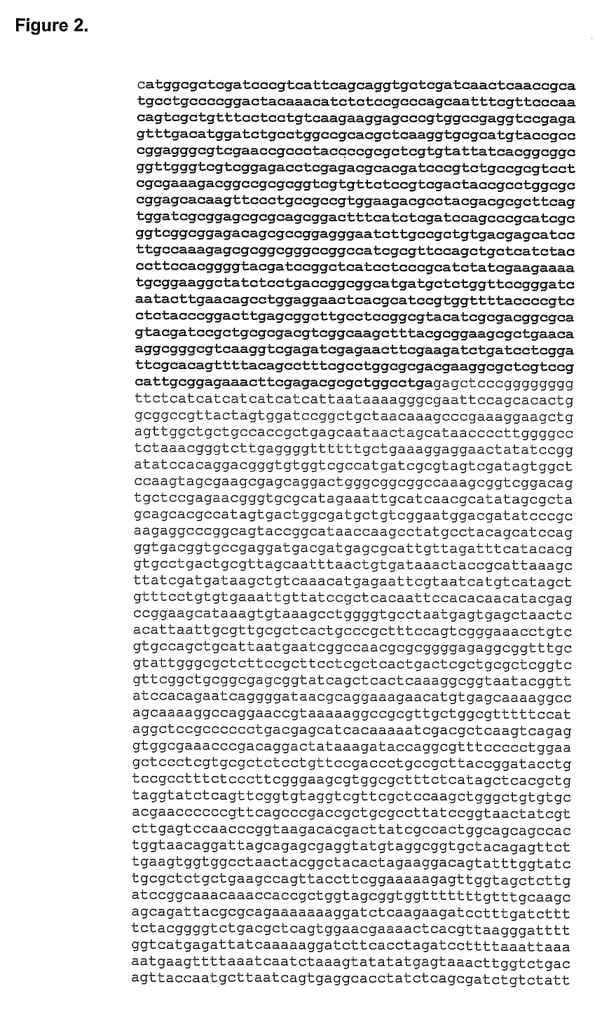

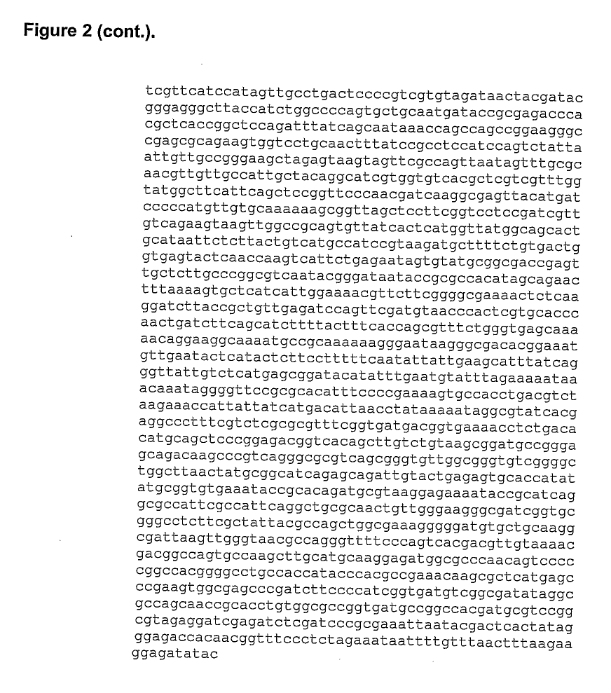

FIG. 2.

Nucleotide sequence of pIVEX 2.3d-Est2 (pEst2; SEQ ID NO: 9). Bolded letters are the coding sequence (SEQ ID NO: 1 of the esterase 2 (Est2; SEQ ID NO: 2 or 62) from Alicyclobacillus acidocaldarius

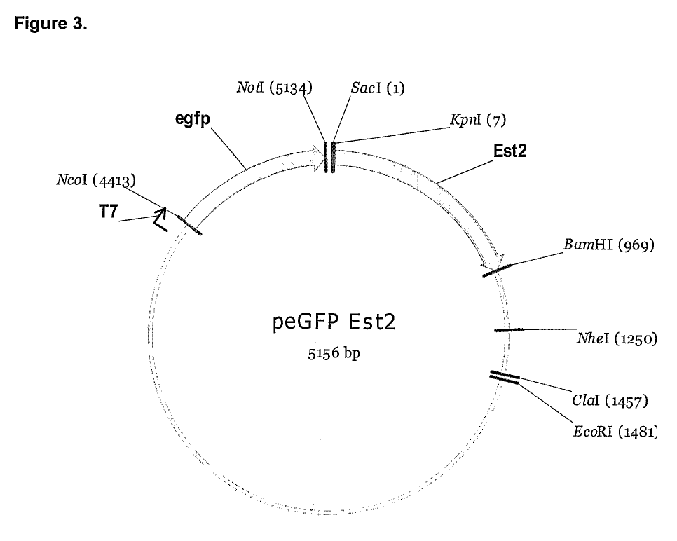

FIG. 3.

Plasmid map of pIVEX_eGFP-Est2 (peGFP-Est2).

FIG. 4.

Nucleotide sequence of pIVEX_eGFP-Est2 (peGFP-Est2; SEQ ID NO: 8). Bolded letters are the coding sequence (SEQ ID NO: 1 of the esterase 2 (SEQ ID NO: 2 or 62), underlined letters are the coding sequence of the enhanced green fluorescent protein.

FIG. 5.

Different substrates an their esterase-catalysed reactions for the (A) electrochemical, (B) optical and (C) fluorescence detection of the esterase activity.

FIG. 6.

In vitro synthesis of the esterase 2 from Alicyclobacillus acidocaldarius in E. coli translation system. The templates for the protein biosynthesis were: open circles, EF-Ts gene, filled squares pEst2; open triangles—control without template.

A: accumulation of newly synthesized protein measured by [¹⁴C]leucine incorporation.

B: activity of the esterase determined by hydrolysis of p-nitrophenyl acetate,

C: activity of the esterase determined by hydrolysis of 5-(and -6)-carboxy-2′,7′-dichlorofluorescein diacetate.

FIG. 7.

SDS-PAGE of the in vitro translated polypeptides. The templates are indicated on the top of corresponding lane. Aliquots of 2 μl translation reaction mixture were withdrawn after 90 min. incubation time. EF-Ts, synthesis of elongation factor Ts from E. coli; Est, synthesis of esterase 2 from Alicyclobacillus acidocaldarius.

A: Coomasie blue-stained polyacrylamide gel,

B: radioactive image of the gel,

C: staining for esterase activity.

Positions of marker proteins and of the esterase 2 are indicated by arrows.

FIG. 8.

Affinity purification of expressed esterase with covalently linked protein.

1: Inhibitor possessing high affinity to esterase is attached via a spacer to a matrix.

2: Esterase covalently linked to a protein is bound to the matrix, residual proteins are washed out.

3a: The esterase is eluted from the column and the amount of the co-purified protein X is determined by the linked esterase activity.

3b: linked peptide X is cleaved and washed of the column.

FIG. 9.

Esterase as an affinity tag for purification of the in vitro synthesized proteins. The in vitro transcription/translation was performed for 90 min. with the pEst2 as a template.

A: radioactive image of the SDS-PAGE of 4 μl samples. The mobilities of eGFP-esterase, esterase and eGFP are indicated by arrows.

B: esterase activity (grey bars) and eGFP fluorescence (black bars) in the samples.

The samples at subsequent purification steps were: 1—translation mixture after 90 min. incubation time; 2—translation mixture after 90 min. incubation followed by treatment with TFK-matrix SDS-PAGE, samples were then taken from the supernatant. 3—material released from the TFK-matrix by treatment with Factor Xa protease, 4—material which remained bound to the TFK-matrix after protease treatment and was released by treatment with 1% SDS at 95° C. After dialysis at 25° C. the activity of the esterase was determined.

FIG. 10.

Scheme for screening of protein biosynthesis inhibitory activity in a cell-free translation assay.

FIG. 11.

Immobilization of Esterase-Puromycin conjugates produced on streptavidin coated glass surface. Translation reaction was performed as described in Experimental section. Plate 1 is a control onto which 1 μL purified esterase, prepared by purification from E. coli cells overexpressing the enzyme (Arkov (2002), J Bacteriol. 184, 5052-5057.). On the plates 2, 3, and 4 one μL translation mixture containing no puromycin derivative, Biotin-CpPuromycin and Biotin-CpPuromycin together with anti-RF1 antibodies, respectively. After 90 min. at 37° C. the plates were rinsed by tap-water and the esterase activity was determined by 2-naphthyl acetate assay and developed with Fast Blue BB salt.

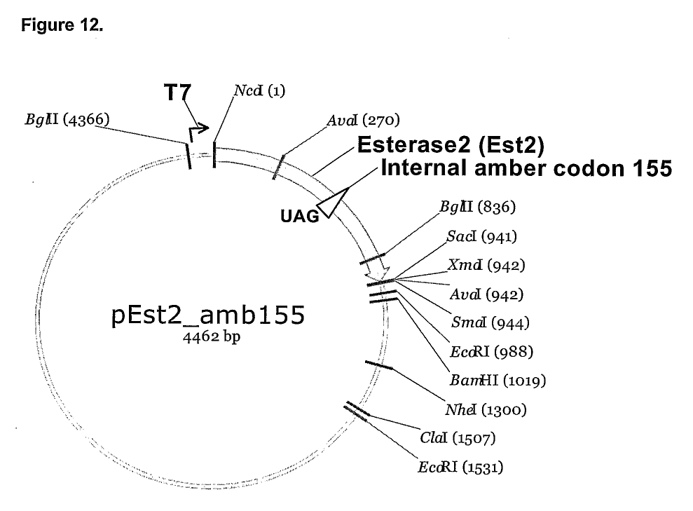

FIG. 12.

Plasmid map of pEst2_amb155.

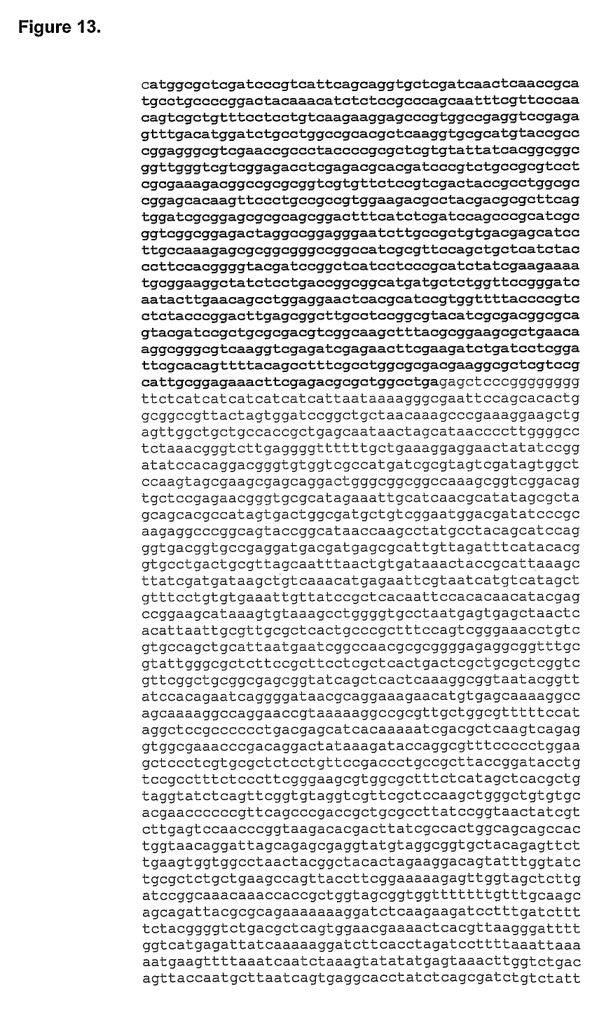

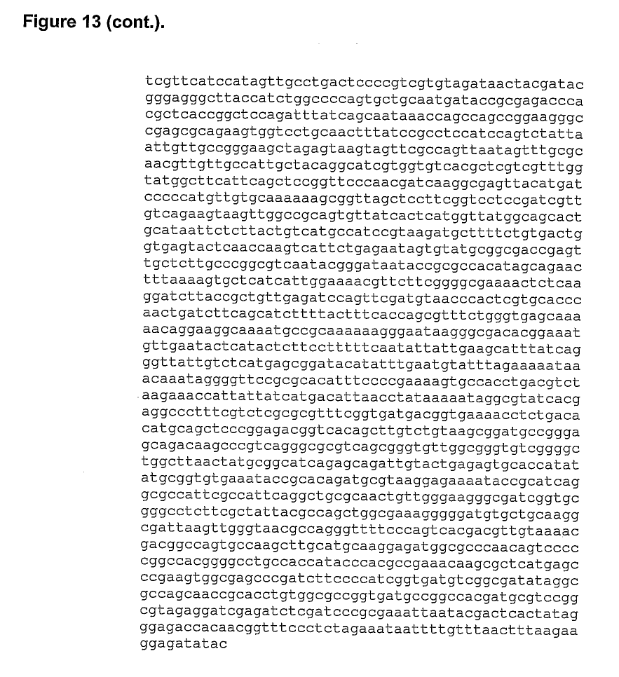

FIG. 13.

Nucleotide sequence of pEst2_amb155 (SEQ ID NO: 10). Bolded letters are the coding sequence of esterase 2 S155 amber mutant.

FIG. 14.

Substitution of the sense codon for catalytically important Serine 155 (AGC) into nonsense codon (UAG) in the mRNA for the esterase 2 from Alicyclobacillus acidocaldarius

A: Scheme of mutated mRNA

B: The esterase catalytic triad (Ser-His-Asp) structural organization (De Simone (2000), J Mol. Biol 303, 761-771).

FIG. 15.

In vitro synthesis of the pEst2_Amb155.

A: Kinetics of in vitro transcription/translation of the pEst2_Amb155; filled triangles: pEst2 (control) as a template; filled squares: pEst2_Amb155 as a template. The concentration of the newly synthesized proteins was determined by measurement of the TCA precipitatable [¹⁴C]leucine radioactivity in aliquots withdrawn at indicated time intervals.

B: Radioactive image of the SDS-PAGE of 2 μl samples from the in vitro translation mixture after 120 min. incubation time. Lane 1-pEst2_Amb155 as a template; lane 2-pEst2 (control) as a template.

C: Esterase activity of the in vitro synthesized polypeptides determined by hydrolysis of p-nitrophenyl acetate. Bar 1-pEst2_Amb155 as a template; bar 2-pEst2 (control) as a template.

FIG. 16.

Suppression of amber stop codon by suppressor Ser-tRNA^{Ser(CUA)} in the presence of antibodies against RF1. Samples from the in vitro translation system programmed by pEst2_Amb155 were withdrawn after 160 minutes of incubation in the presence of [¹⁴C]leucine.

A: Translation was carried out in the absence of anti-RF1 antibodies. Radioactive image of the gel after SDS-PAGE separation of 2 μl samples from the in vitro translation mixture is presented. Concentration of added suppressor tRNA^{Ser(CUA)} was as follows: lane—no tRNA^{Ser} added, lane 2—24 nM, lane 3—120 nM, lane 4-600 nM, lane 5—2.5 μM, lane 6—10 μM, lane 7—25 μM.

B: Translation was carried out in the presence of anti-RF1 antibodies. Radioactive image of the gel after SDS-PAGE separation of 2 μl samples from the in vitro translation mixture is presented. Concentration of added suppressor tRNA^{Ser} was as described in (A).

C: Enzymatic activity of the in vitro produced esterase determined by hydrolysis of p-nitrophenyl acetate. Samples of 1 μl were used for detection. Grey bars, translation was carried out in the absence of anti-RF1 antibodies, black bars, translation was carried out in the presence of anti-RF1 antibodies. Concentration of added suppressor tRNA^{Ser(CUA)} was as described in (A).

FIG. 17.

Plasmid map of pNox-Est2.

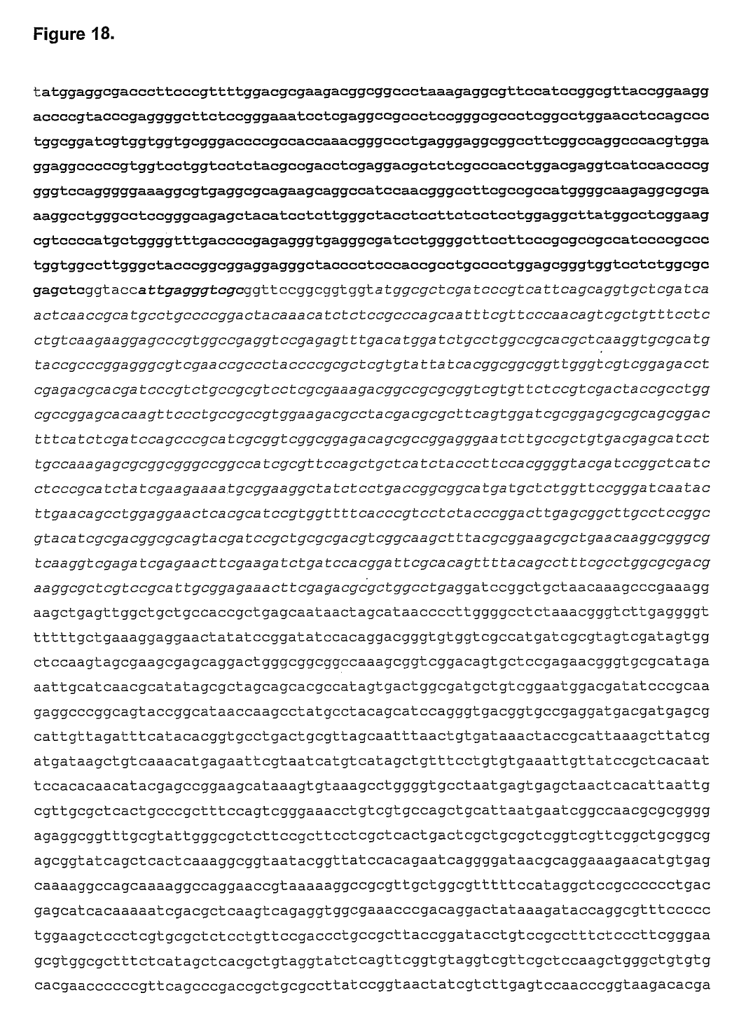

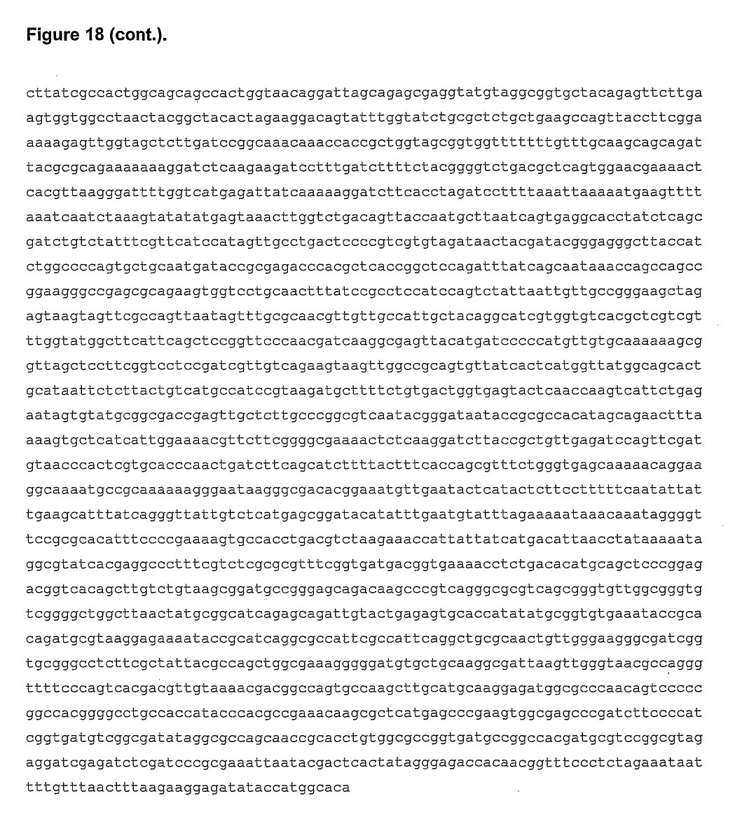

FIG. 18.

Nucleotides sequence of pNox-Est2 (SEQ ID NO: 11).

Bold letters are gene of Nox. Italic letters are gene of Est2 (SEQ ID NO: 1). Bold and italic letters are the coding sequence of Factor Xa cleavage site.

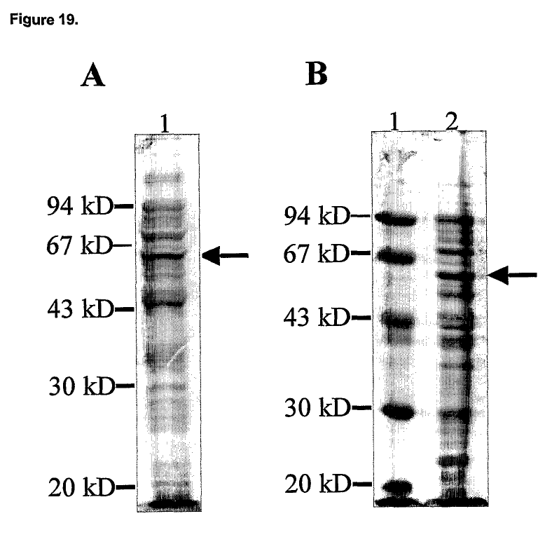

FIG. 19.

SDS-PAGE of in vitro and in vivo expression of Nox-Est2 fusion protein with Esterase activity staining (bands marked by arrows) and coomassie blue G-250 staining of total protein.

A: in vitro expression of Nox-Est2 fusion protein. The position of the Nox-Est2 fusion protein is indicated by arrow.

B: in vivo expression of Nox-Est2 fusion protein. Lane 1: protein molecular weight standard; lane 2: cell extracts. Nox-Est2 fusion protein is indicated by black arrow.

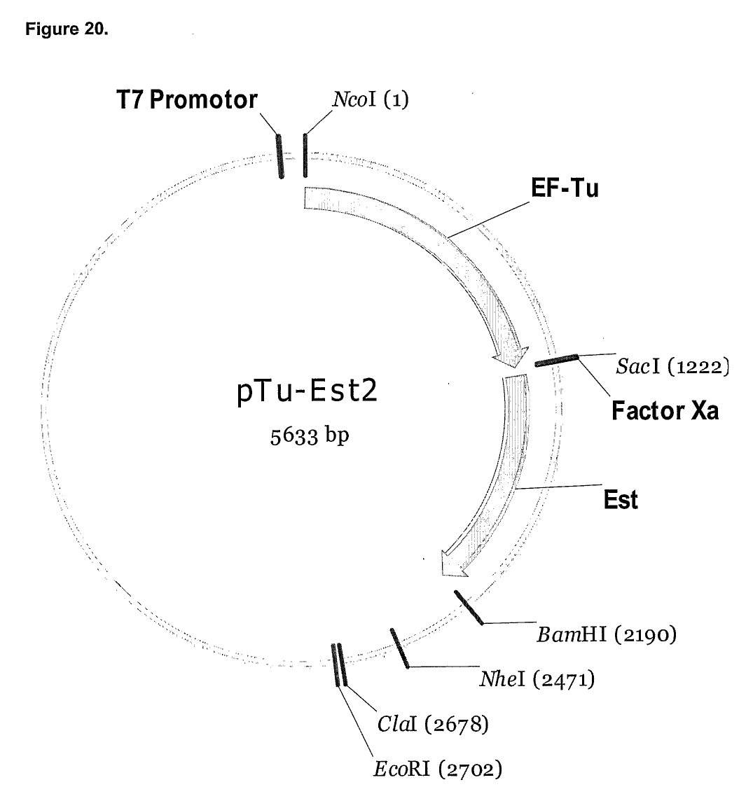

FIG. 20.

Plasmid map of pTu-Est2.

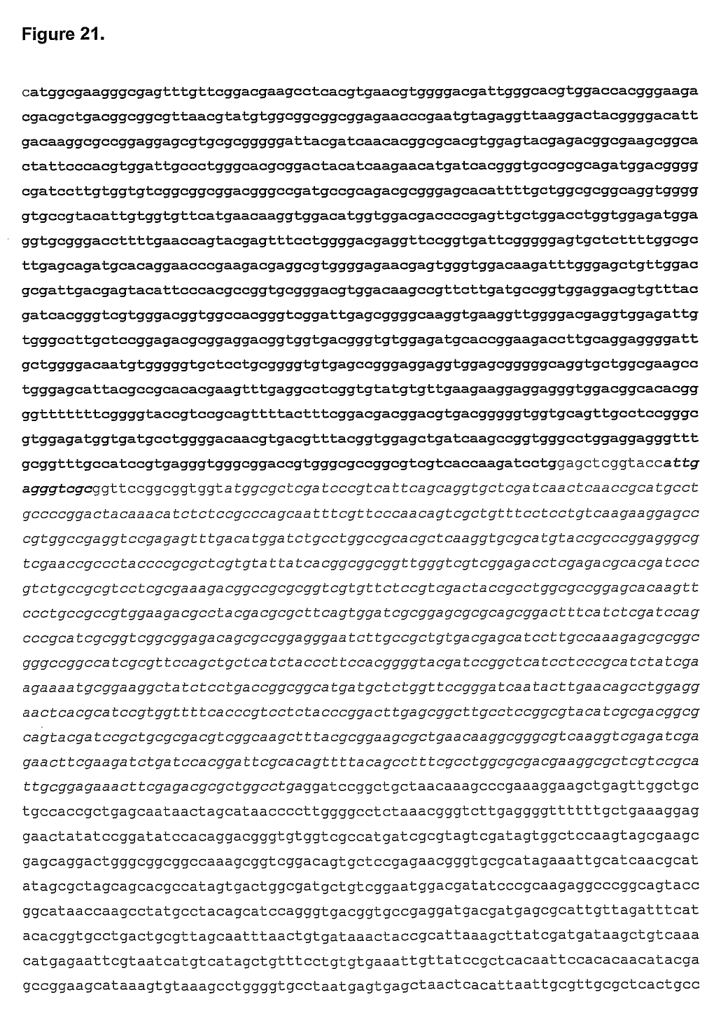

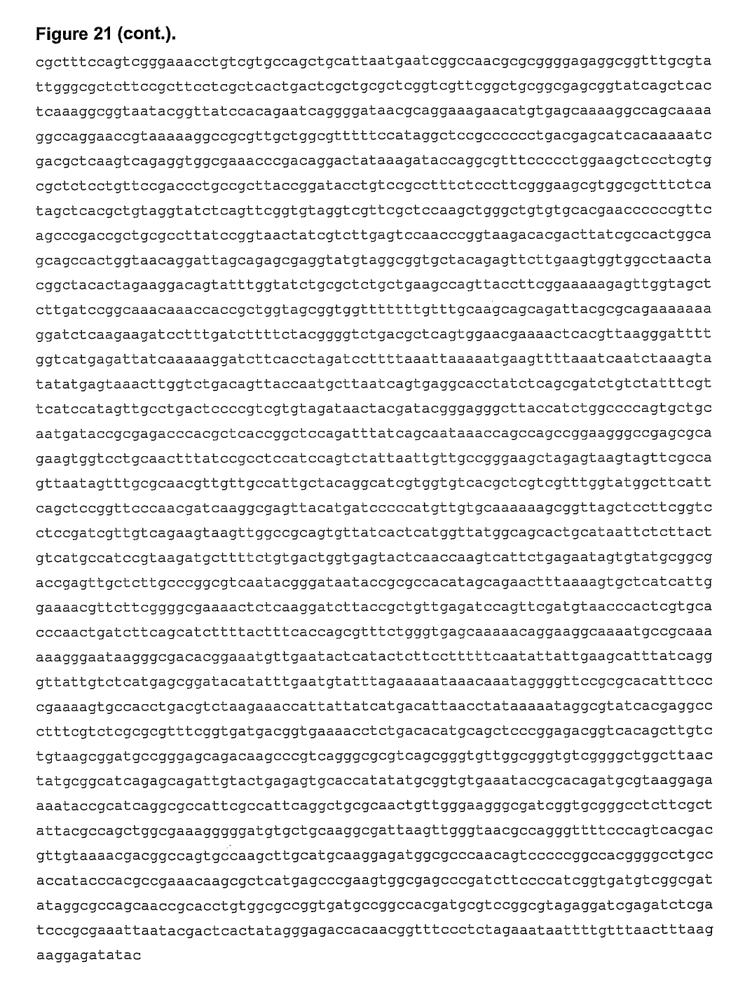

FIG. 21.

Nucleotides sequence of pTu-Est2 (SEQ ID NO: 12).

Bold letters are gene of EF-Tu. Italic letters are gene of Est2 (SEQ ID NO: 1). Bold and italic letters are the coding sequence of Factor Xa cleavage site.

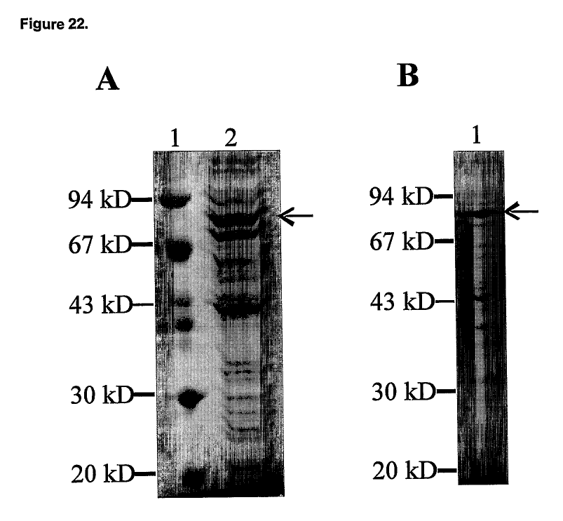

FIG. 22.

SDS-PAGE of in vitro and in vivo expression of EF-Tu-Est2 fusion protein with Esterase activity staining and protein coomassie blue G-250 staining.

A: in vitro expression of EF-Tu-Est2 fusion protein. Lane 1: protein molecular weight standard; Lane 2: in vitro expression mixture. EF-Tu-Est2 fusion protein was pointed out by the black arrow.

B: in vivo expression of EF-Tu-Est2 fusion protein. EF-Tu-Est2 fusion protein was pointed out by the black arrow.

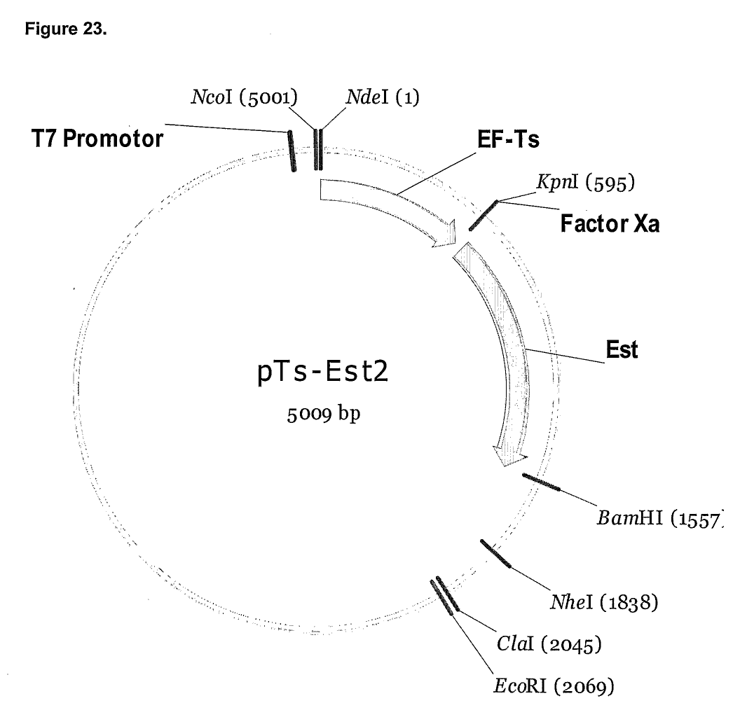

FIG. 23.

Plasmid map of pTs-Est2.

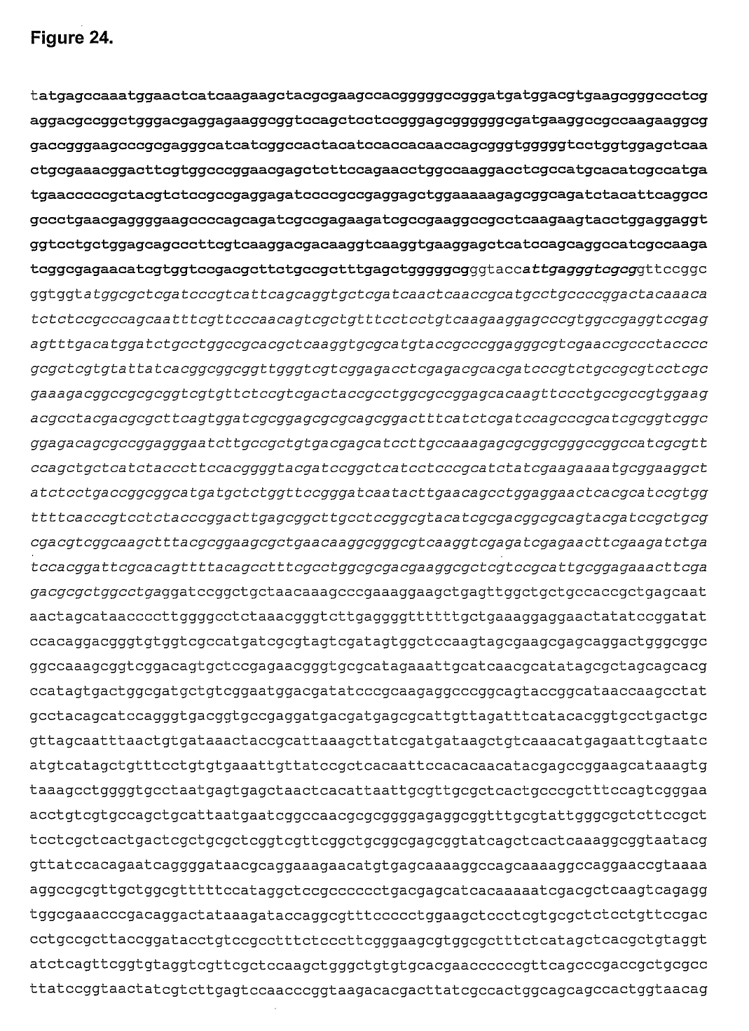

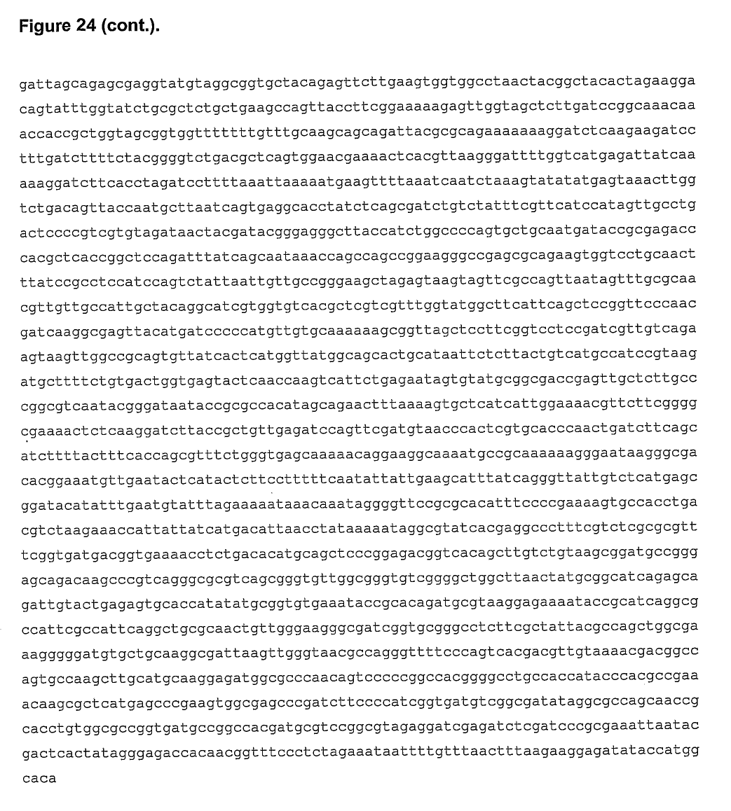

FIG. 24.

Nucleotides sequence of pTs-Est2 (SEQ ID NO: 13).

Bold letters are gene of EF-Ts. Italic letters are gene of Est2 (SEQ ID NO: 1). Bold and italic letters are the coding sequence of Factor Xa cleavage site.

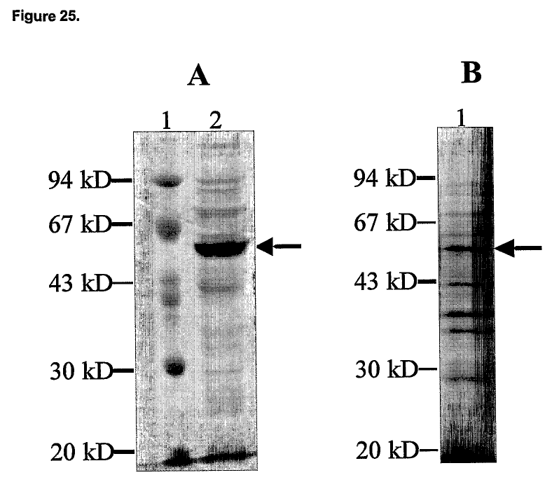

FIG. 25.

SDS-PAGE of in vitro and in vivo expression of EF-Ts-Est2 fusion protein using esterase activity and protein coomassie blue G-250 staining.

A: in vitro expression of EF-Ts-Est2 fusion protein. Lane 1: protein molecular weight standard; lane 2: in vitro expression mixture. EF-Ts-Est2 fusion protein is indicated by a black arrow.

B: in vivo expression of EF-Ts-Est2 fusion protein. EF-Ts-Est2 fusion protein is indicated by a black arrow.

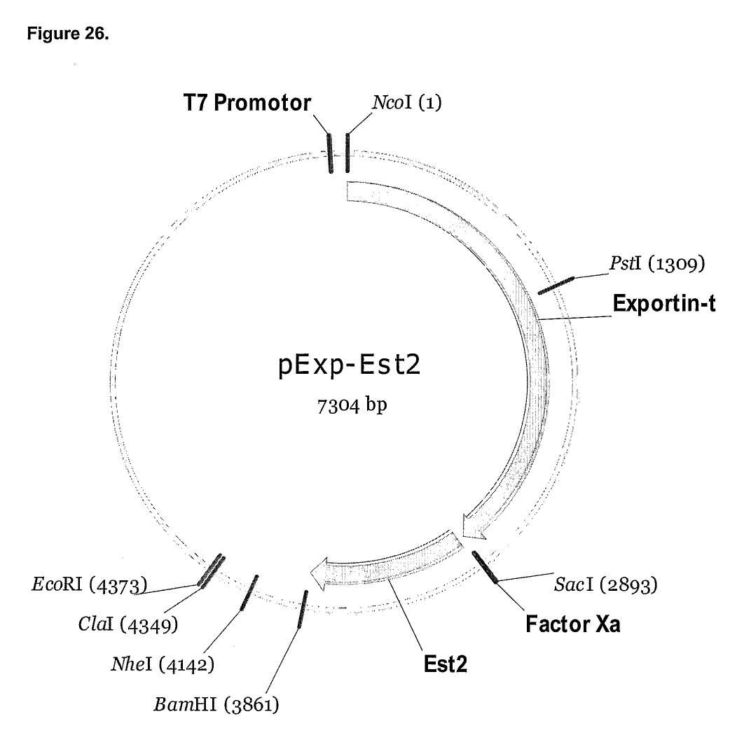

FIG. 26.

Plasmid map of pExp-Est2.

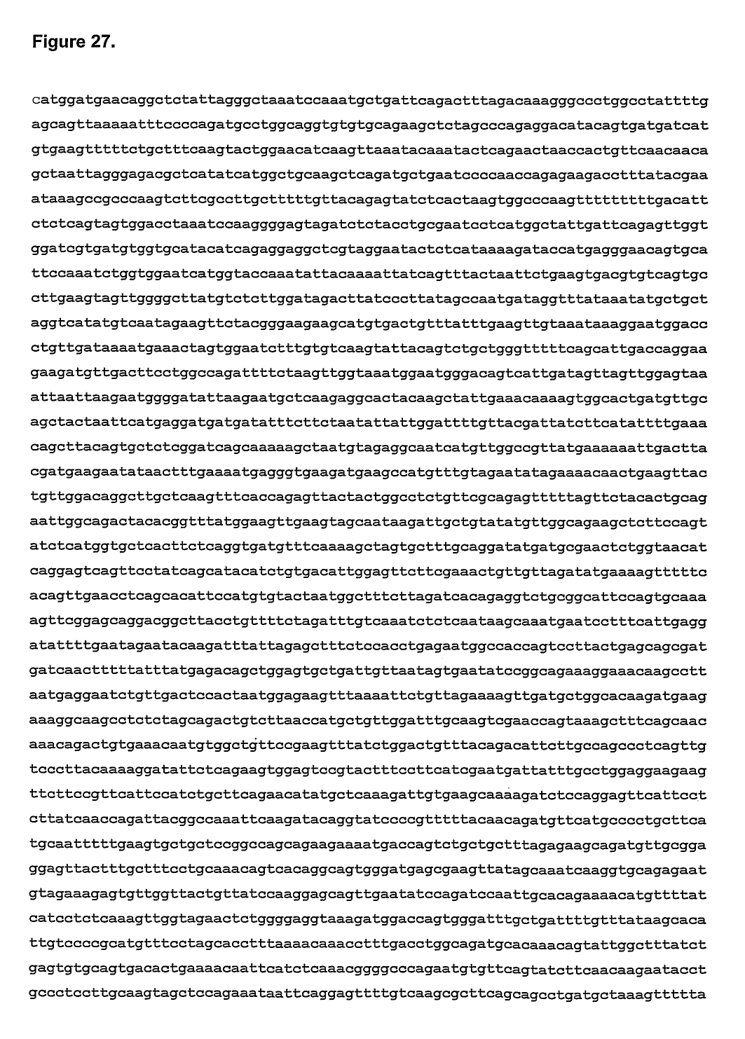

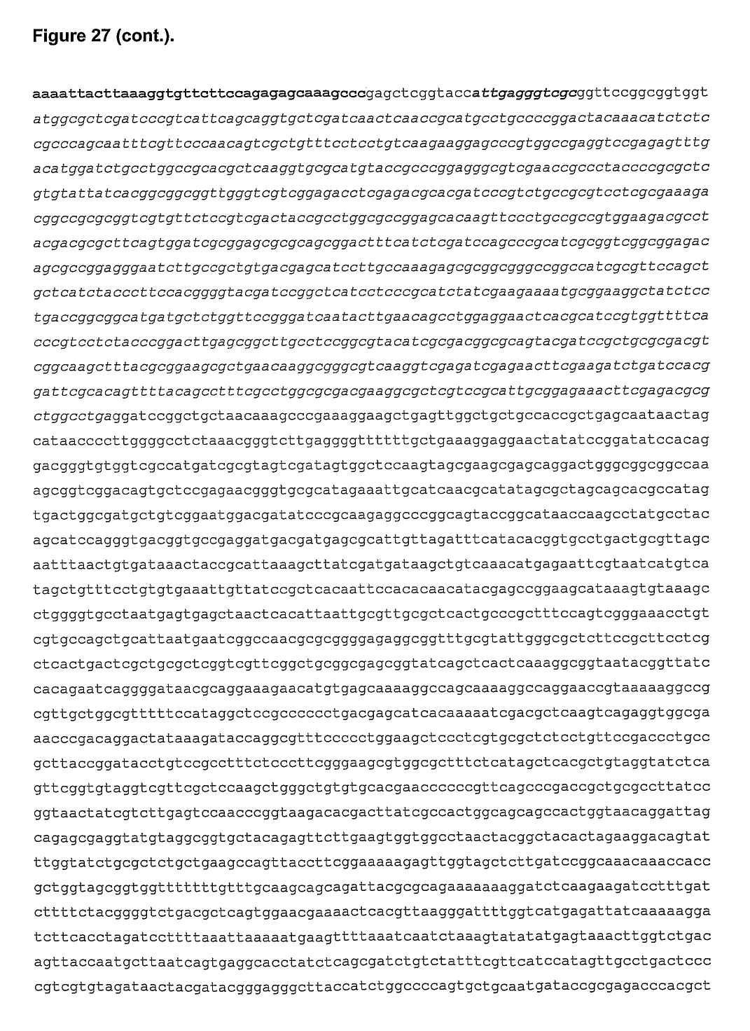

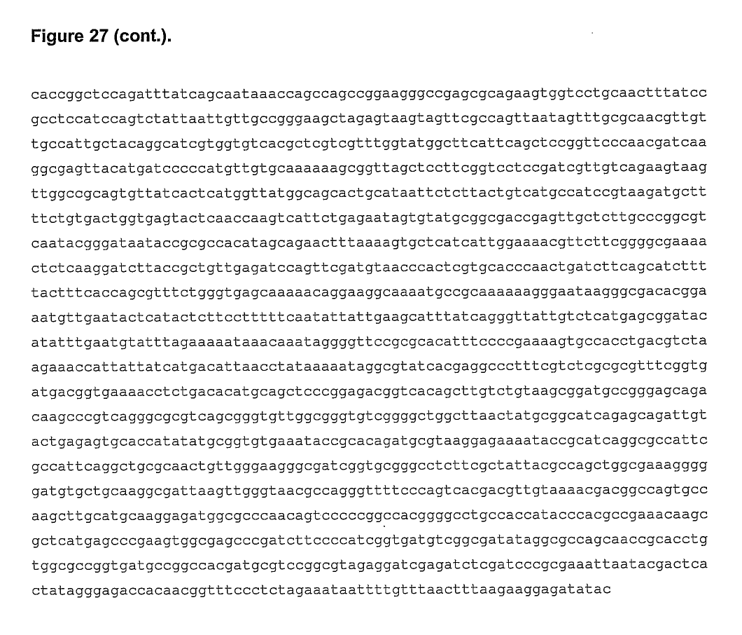

FIG. 27.

Nucleotides sequence of pExp-Est2 (SEQ ID NO: 14).

Bold letters are a gene of Exportin-t. Italic letters are agene of Est2 (SEQ ID NO: 1).

Bold and italic letters are the coding sequence of Factor Xa cleavage site.

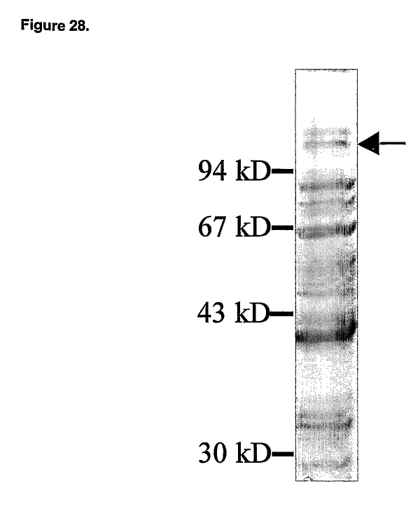

FIG. 28.

SDS-PAGE of in vitro expression of Exportin-t-Est2 fusion protein. The fusion protein detected by enzymatic staining for esterase is indicated by arrow.

Positions of protein molecular weight standard was indicated by bars the total protein bands were stained by coomassie blue G-250.

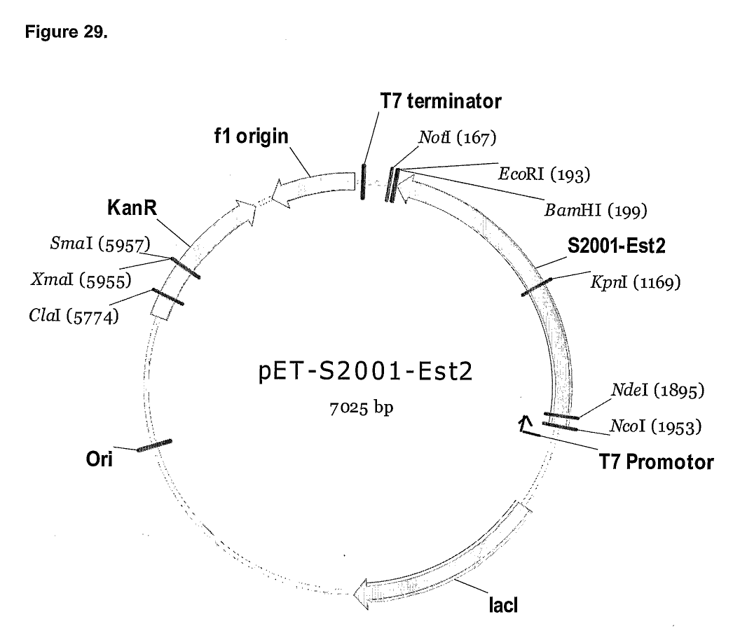

FIG. 29.

Plasmid map of pET-S2001-Est2.

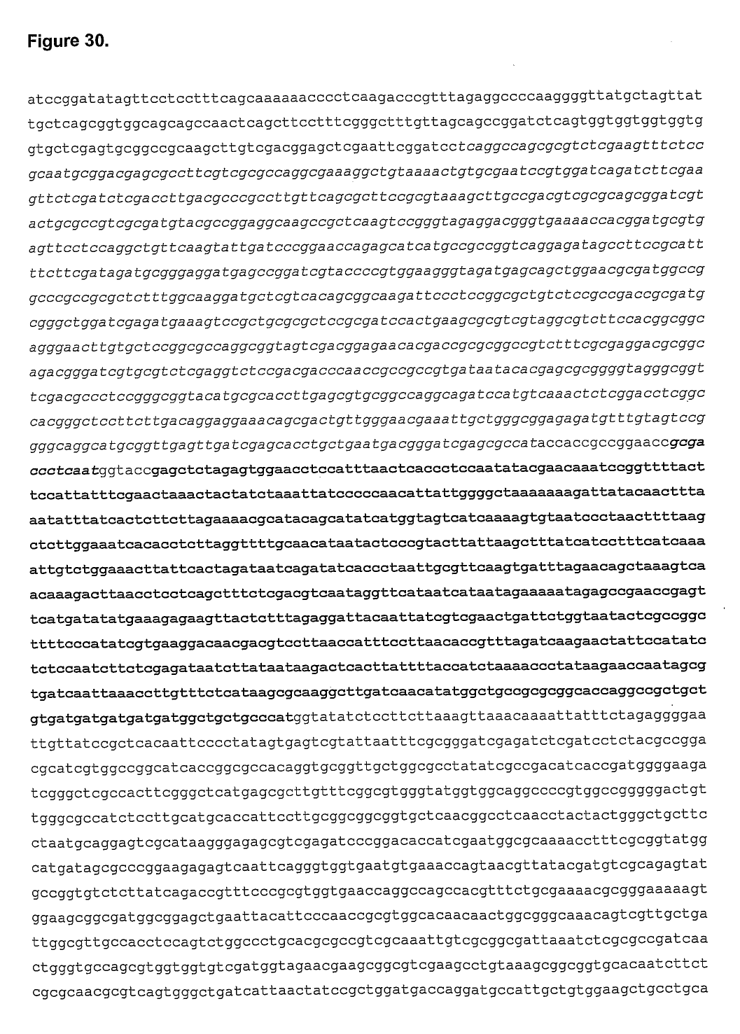

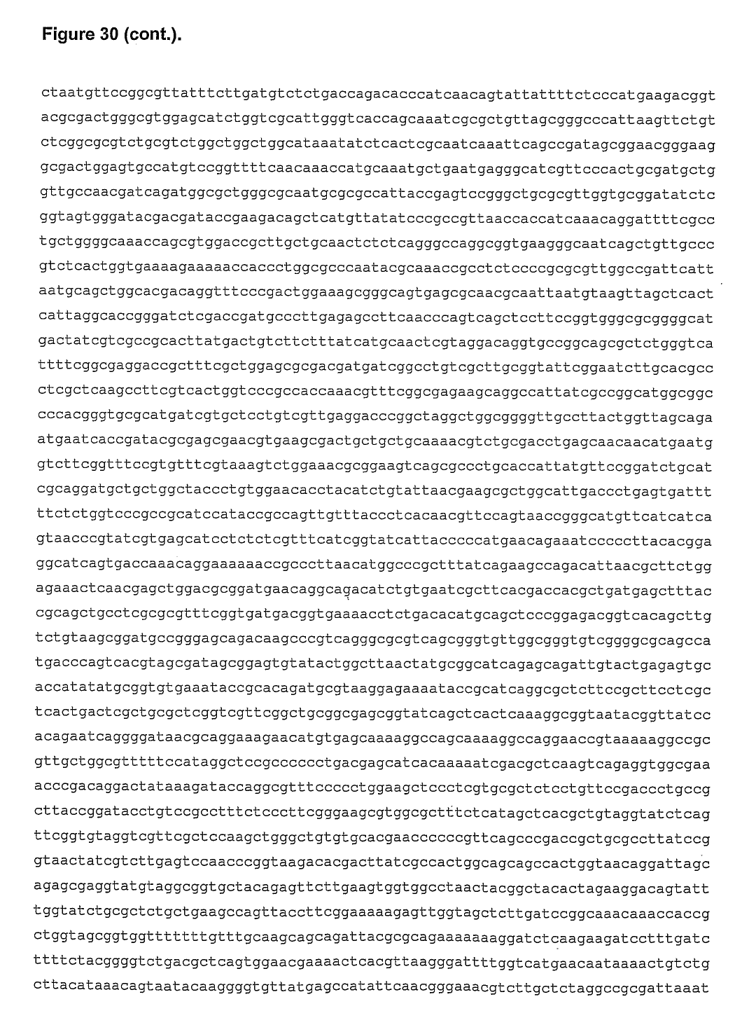

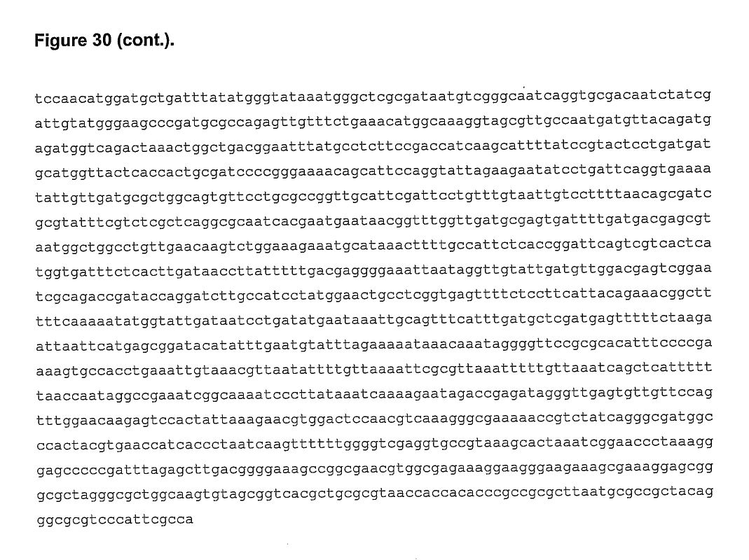

FIG. 30.

Nucleotides sequence of pET-S2001-Est2 (SEQ ID NO: 15).

Bold letters are gene of putative nuclease S2001. Italic letters are gene of Est2 (SEQ ID NO: 1). Bold and italic letters are the coding sequence of Factor Xa cleavage site.

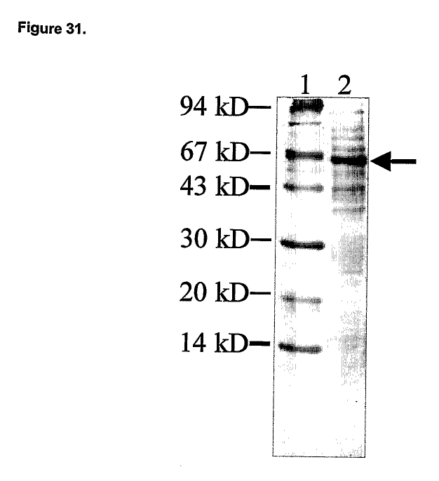

FIG. 31.

SDS-PAGE of in vivo expression of S2001-Est2 fusion protein with Esterase activity and protein coomassie blue G-250 staining.

Lane 1: protein molecular weight standard; lane 2: cell extract of in vivo expression of S2001-Est2 fusion protein. The S2001-Est2 fusion protein is indicated by an arrow.

FIG. 32.

Purification of recombinant Nox-Est2 fusion protein on TFK-Sepharose.

a: SDS-PAGE of: (1) molecular weight standards, (2) proteins in the break-through volume of the Nox-Est2 fusion protein overexpressing E. coli cellular extract, (3) Nox-Est2 fusion protein eluted by 1,1,1-Trifluoro-3-(2-hydroxy-ethylsulfanyl)-propan-2-one (F₃C—CO—CH₂—S—CH₂—CH₂—OH). The arrow indicate the position of Nox-Est2 fusion protein, the bars the molecular masses of protein standards. The gels were stained by coomassie blue and esterase activity staining.

b: Purification of recombinant Nox-Est fusion protein. SDS-PAGE of samples from different steps of NADH oxidase purification. The positions of the molecular weight standards are shown by bars. After SDS-PAGE, the polyacrylamide gel was treated by activity staining of esterase, then stained with coomassie blue G-250. Lane 1: cell extracts; lane 2: break-through peak; lane 3: Nox eluted from TFK column via amino acid sequence specific cleavage by Factor Xa; Lane 4: Factor Xa from commercial source. The arrow indicate the position of NADH oxidase (Nox).

The Examples illustrate the invention.

### EXAMPLE 1

### Preparation of the Plasmid pIVEX 2.3d-Est2 (pEst2) Comprising a cDNA Encoding the Esterase 2 (Est2) from Alicyclobacillus acidocaldarius

Plasmid pT7SCII containing esterase 2 (Est2) was kindly provided by G. Manco, Napples, Italy (Manco (1998), Biochem. J, 332 (Pt 1), 203-212.). The Est2 gene was amplified by using pT7SCII-esterase as template, recombinant Taq-polymerase, and two synthetic oligonucleotides,

estfor (5′-CCATGGCGCTCGATCCCGTCATTCAGC-3′; SEQ ID NO: 16) and estrev (5′-GAGCTCCTAGGCCAGCGCGTCTCG-3′; SEQ ID NO: 17) in a 30-cycle polymerase chain reaction (1 min at 95° C., 30 sec 60° C., and 1 min at 72° C.).

The primer estfor was designed to introduce a NcoI restriction site (underlined) at the initiation site which also leads to a C to G exchange (bold) in the coding sequence (proline at position 2 changes to alanine). This amino acid replacement has no effect on structure or function of the enzyme. Primer estrev introduces a SacI restriction site (underlined) downstream from the UAG stop codon (bold). The PCR product was eluted from an agarose gel and ligated into pGEM-T vector (Promega) and completely sequenced to verify that only desired mutations were introduced. The obtained plasmid was then digested with NcoI and SacI, the cloned fragment was eluted from an agarose gel and ligated into NcoI-SacI-linearized in vitro-translation-vector pIVEX2.3d (Roche Diagnostics, Mannheim, Germany). The resulting plasmid, pIVEX2.3d-Est2 (pEst2), was used for in vitro translation. A map of pEst2 is shown in FIG. 1, the corresponding nucleotide sequence is shown in FIG. 2 (SEQ ID NO: 9).

### EXAMPLE 2

### Preparation of the Plasmid pIVEX2.3d-eGFP-Est2 (peGFP-Est2) Comprising a cDNA Encoding a Fusion Protein Comprising eGFP, Esterase 2 (Est2) from Alicyclobacillus acidocaldarius and a Cleavable Linker

The pIVEX2.3d-eGFP-Est2 (peGFP-Est2) plasmid was constructed as follows. The gene of enhanced green fluorescence protein (eGFP) was amplified from the plasmid pSL1180-eGFP (Genbank, accession number pEGFP-1-U55761, provided by G. Krauss, Bayreuth) by PCR with the primers eGFP_for (5′-CCATGGTGAGCAAGGGCG-3′; SEQ ID NO: 18) and eGFP_rev (5′-GCGG CCGCCTTTGTACAGCTCGTCCAT-3′; SEQ ID NO: 19). The primers introduce the NcoI and the NotI cleavage sites (underlined letters) upstream and downstream of the eGFP, respectively. The PCR product was sequenced and cloned into the pIVEX2.3d vector resulting in the pIVEX2.3d-eGFP (peGFP) plasmid. The Est2 was amplified with primers Est2CT_for (5′-GAGCTCGGTACCATTGAGGGTCGCGGTTCCGGCGGTGGTATGGCGCTCGATC CC-3′; SEQ ID NO: 20) and Est2CT rev (5′-GGATCCTCAGGCCAGCGC-3′; SEQ ID NO: 21). The primers create the SacI and the BamHI cleavage sites (underlined letters) upstream and downstream of the Est2, respectively. The primer Est2CT_for contains the cleavage site of protease Factor Xa coding sequence (bold letters) and the primer Est2CT_rev contains the UAG stop codon (bold letters). The PCR product was sequenced and cloned into the peGFP plasmid. The resulting plasmid, peGFP-Est2, was used for in vitro translation. A map of peGFP-Est2 is shown in FIG. 3, the corresponding nucleotide sequence is shown in FIG. 4 (SEQ ID NO: 8).

### EXAMPLE 3

### Purification of the Plasmids pEst2 and peGFP-Est2

The plasmids for coupled in vitro transcription/translation were purified with modified PEG method (Nicoletti (1993), Biotechniques, 14, 532-4, 536.). Therefor, the 12.5 ml cell culture, containing the plasmid was harvested and resuspended in 240 μL of 25 mM Tris/HCl pH 8.0, 50 mM glucose, 10 mM EDTA. Then 600 μL of 0.2 N NaOH, 1% (w/v) SDS was added. The tube was gently turned over for several times and incubated for 4 minutes at room temperature. 450 μL of 3.6 M NaOAc, pH 5.0 was added and the suspension was gently mixed by turning over the tube for 20 times and incubated for 4 minutes at room temperature. Cell debris was removed by centrifugation at 16,000 g for 10 minutes and the supernatant was mixed with 400 μL of 40% PEG 6000 and kept on ice for one hour. The sample was centrifuged at 16,600 g for 10 minutes and the pellet was completely dissolved in 150 μL ddH₂O. After addition of 300 μL of saturated NH₄Ac the suspension was incubated for 15 minutes on ice and then centrifuged at 16,600 g for 10 minutes at room temperature. The supernatant was mixed with 300 μL of isopropanol and incubated for 15 minutes at room temperature. After centrifugation at 16,600 g for 10 min, the DNA pellet was washed two times with 75% ethanol, dried and dissolved in distilled water.

### EXAMPLE 4

### The SDS-PAGE of the Present Invention

Protein pattern of the reaction mixture was analysed by SDS-PAGE (Schagger (1987), Anal. Biochem., 166, 368-379.). Aliquots were mixed with the sample buffer, incubated 5 min. at 95° C. and loaded onto 10% polyacrylamide gel. After the run the gels were fixed with 15% formaldehyde in 60% methanol and stained with Coomassie Blue G-250.

For imaging radioactivity, the dried gels were exposed to an imaging plate for radioactivity analysis with the Phosphorimager SI (Molecular Dynamics, Sunnyvale, USA).

Activity staining of the esterase in polyacrylamide gels after electrophoretic separation was performed according to (Higerd (1973), J. Bacteriol., 114, 1184-1192.) with Fast Blue BB Salt and β-naphthyl-acetate.

### EXAMPLE 5

### The Esterase Activity Assays of the Present Invention

Determination of esterase activity was performed as described (Manco (1998), Biochem. J., 332, 203-212.) with minor modifications.

Aliquots of 1 μl transcription/translation mixture were added to 1 ml of 50 mM phosphate buffer pH 7.5 containing 0.025 mM p-nitrophenyl acetate. The production of p-nitrophenoxide was monitored at 405 nm in 1 cm path-length cells with UV-Spectral photometer DU 640 (Beckman, Fullerton, USA) at 25° C. Initial rates were calculated by linear least-square analysis of time courses comprising less than 10% of the total substrate turnover.

Esterase activity was also determined by fluorescence assay. At each time interval 1 μl was withdrawn from transcription/translation mixture and added to 1 ml of 50 mM phosphate buffer pH 7.5 with 0.025 mM 5-(and -6)-carboxy-2′,7′-dichlorofluorescein diacetate. Production of 5-(and -6)-carboxy-2′,7′-dichlorofluorescein was measured at 25° C. by Luminescence spectrometer LS50B (Perkin Elmer, Boston, USA) with λₑₓ at 500 nm and λₑₘ at 525 nm.

Staining of esterase activity in polyacrylamide gels was performed as described herein above (Example 4).

### EXAMPLE 6

### In Vitro Synthesis of the Esterase 2 (Est2) from Alicyclobacillus acidocaldarius in an E. coli Cell-Free Translation System

Esterase 2 from Alicyclobacillus acidocaldarius was synthesized by coupled in vitro transcription/translation system derived form E. coli. Although, the codon usage of the esterase gene was not adjusted to the codon usage of E. coli, the synthesis of the thermostable esterase proceeds with similar efficiency in this heterologous system as the synthesis of one most abundant E. coli proteins, the elongation factor Ts. FIG. 6 demonstrates the in vitro [¹⁴C]leucine incorporation into the esterase (FIG. 6A) with the simultaneous monitoring of the esterase activity (FIG. 6B). The system produces the target proteins linearly up to 60 minutes of incubation. The estimated yield for the EF-Ts and the esterase was approximately 350 and 200 micrograms of the protein per 1 ml of the reaction mixture, respectively (FIG. 6A). The in vitro produced esterase possesses high enzymatic activity (FIG. 6B). Thus, even thousand fold dilution of the in vitro synthesized esterase in the assay mixture, which results in 10⁻⁸ M final esterase concentration, provides well-detectable initial rates of the enzymatic activity. In contrast, the level of esterase activity in the absence of esterase gene is very close to the background (FIG. 6B) providing evidence for the absence of endogenous E. coli esterase activity in the translation system. Besides the standard photometric detection the fluorescence measurement of the esterase activity was carried out. Enzymatic hydrolysis of the 5-(and -6)-carboxy-2′,7′-dichlorofluorescein diacetate by the esterase leads to appearance of the fluorescent product (FIG. 6C). Kinetics of esterase production detected by fluorescence coincide with ones determined photometrically or by polypeptide-incorporated radioactivity.

The SDS-PAGE of the total protein from the reaction mixture with subsequent detection of radioactivity distribution and staining of the gel for esterase activity were also performed for in vitro synthesized esterase in comparison with EF-Ts. The protein samples analyzed by SDS-PAGE contain many endogenous E. coli proteins (FIG. 7A), which are, however, not labeled with [¹⁴C]leucine (FIG. 7B). The autoradiogram of the gel (FIG. 7B) reveals distinct bands at the position of the esterase (MW ˜34.4 kD) and of the control protein EF-Ts (MW ˜31.6 kD) with more than 90% of incorporated radioactivity belonged to the full-length products in both cases. The faster migrating bands are probably incomplete polypeptides translated from truncated mRNAs. The in situ activity staining of the esterase in polyacrylamide gel detects only one band that corresponds to the full-length esterase. This is in contrast with the lack of esterase activity in the lines related to EF-Ts and in the control without template (FIG. 7C).

The kits used for in vitro translation experiments within the present application were evaluation size transcription/translation kits from RiNA GmbH, (Berlin, Germany, kindly provided by Dr. W. Stiege) and the reaction was performed at 37° C. according to the manual provided by the supplier. [¹⁴C]Leucine (17.3 mCi/mmol) was added up to 0.5 mM. The template (control vector, peEst2 or peGFP-Est2) was added up to 5 nM. The reaction was started by transferring the reaction tube to the thermo shaker at 37° C. with 500 rpm agitation. Aliquots of 3 μl were withdrawn at different time intervals (up to 2 hours) and the newly synthesized protein was determined by radioactivity measurement in 10% trichloroacetic acid precipitate.

As an example, the detailed protocol of the in vitro translations performed within the present application is listed below.

Protocol for an in vitro translation (RiNA GmbH Kits):

For preparation of a 30 μl reaction mixture the following components should be combined on ice (given in order of mixing):

1. 5.1 μl of 1 mM [¹⁴C]Leu (54 mCi/mmol, Amersham)
2. 1 μl 10 mM Leu (supplied with the Kit)
3. 0.5 μl of RNase free water (supplied with the Kit)
4. 2.4 μl of E-mix (red lid, supplied with the Kit)
5. 9 μl of T-mix without Leu (blue lid, supplied with the Kit)
6. 10.5 μl of S-mix (yellow lid, supplied with the Kit)
7. 1.5 μl of the 100 nM template plasmid

The reaction mixture should be incubated at 37° C. for up to 2 hours with agitation (500 rpm). Aliquots can be withdrawn at any desired time intervals. The reaction can be performed without radioactivity (Leu should be substituted with the same amount of water and T-mix with Leu (supplied with the Kit) should be used instead of one without).

For instance, the in vitro translation system (without RF depleting agents (e.g. Antibodies against RF1 from Thermus thermophilus) and nonsense codon suppressing agents (e.g. puromycin derivatives and/or suppressor tRNAs) of the protocol listed above comprises the following ingredients:
30 S cell-free extract from E. coli (enzyme- and und ribosomal fraction);
MgCl₂ 9-12 mM;
DTT 10 mM;
Amino acids, 200 μM each (For labelling, each amino acid can be applied as a 14C amino acid with a concentration of 100 μM (e.g. 14C-leucine))
Rifampicin 0.02 mg/ml reaction mixture,
Bulk-tRNA 600 μg/ml reaction mixture,
ATP, CTP, GTP, UTP, 1 mM each,
Phosphoenolpyruvate 10 mM;
Acetylphosphate 10 mM;
Pyruvatekinase 8 μg/ml reaction mixture;
Plasmid 2 pmol/ml reaction mixture;
T7 Polymerase 500 Units/ml reaction mixture;
HEPES pH 7.6, 50 mM;
Potassium acetate 70 mM;
Ammonium chloride 30 mM;
EDTA pH 8.0, 0.1 mM;
Sodium azide 0.02%;
Polyethyleneglycol 4000 2%;
Protease inhibitors: aprotinin 10 μg/ml reaction mixture, leupeptin 5 μg/ml reaction mixture, pepstatin 5 μg/ml reaction mixture; and
Folic acid 50 μg/ml reaction mixture.

### EXAMPLE 7

### Affinity Purification of the eGFP-Esterase Fusion Protein by Immobilization on a TFK-Coated Matrix

Esterase 2 from Alicyclobacillus acidocaldarius can be used as an affinity tag for purification of protein esterase fusions (FIG. 8). This is demonstrated in experiments presented in FIG. 9. The synthesis of eGFP-esterase fusion protein is demonstrated by SDS-PAGE and autoradiography of the [¹⁴C]leucine labeled protein (FIG. 9A, lane 1). Trifluoromethyl-alkyl ketones are efficient competitive inhibitors of the esterases with the inhibition constant in μM range. Immobilized trifluoromethyl-alkylketones can be, therefore, used for affinity purification of esterases (Hanzlik (1987), J. Biol. Chem., 262, 13584-13591.). After addition of TFK-Sepharose to translation mixture the eGFP-esterase is almost completely removed from the supernatant (FIG. 9A, lane 2). Cleavage of eGFP from affinity matrix was achieved via the build-in protease sensitive linker by Factor Xa protease. Therefore, after this step the eGFP polypeptide appears in the supernatant (FIG. 9A; lane 3). For release of esterase from affinity matrix harsh conditions (95° C., 1% SDS) had to be used. In the FIG. 9B the esterase activity and florescence of eGFP are demonstrated in different fractions of the affinity-purification steps. The synthesized fusion protein possess both activities (FIG. 9B; bar 1). After treatment with the TFK-Sepharose the supernatant has strongly diminished esterase activity and low eGFP fluorescence due to immobilization of the fusion protein (FIG. 9B; bar 2). After protease cleavage the fluorescent eGFP appears in the supernatant whereas the esterase, as expected, remains attached to the matrix. Correspondingly, no esterase activity can be detected in the supernatant after protease treatment (FIG. 9B; bar 3).

For affinity purification of the eGFP-Esterase fusion protein, the peGFP-Est2 plasmid was expressed in vitro as described above. The fluorescence at 507 nm of eGFP-esterase fusion protein was monitored at λₑₓ=488 nm and 25° C. directly in the translation mixture without dilution using a 150 μl quartz cell. The esterase activity was monitored by photometric assay in parallel with eGFP fluorescence assay. Then 200 μl of the translation mixture was incubated with 25 μl of TFK-matrix (trifluoromethyl ketone Sepharose CL-6B, prepared as described (Hanzlik (1987), J. Biol. Chem., 262, 13584-13591.)) equilibrated with 100 mM Na-phosphate, pH 7.5 at 37° C. for 4 hours. The TFK-matrix was spun down and the supernatant was analyzed for the eGFP fluorescence and the esterase activity. The remaining pellet of TFK-matrix was washed with 3 ml of 100 mM Na-phosphate pH 7.5 with 100 mM NaCl. Then the TFK-matrix was resuspended in 175 μl of 40 mM Tris, 200 mM NaCl, 4 mM CaCl₂, pH 8.0 and treated with 20 μg Factor Xa protease for 15 h at 23° C. The TFK-matrix was spun down and the supernatant was analyzed for the eGFP fluorescence and the esterase activity. The remaining material was removed from TFK-matrix by boiling it for 5 min at 95° C. in 10% SDS. The aliquots from each step of purification were also analysed by SDS-PAGE.

### EXAMPLE 8

### Cloning of the Release Factor 1 of Thermus thermophilus (T.th.RF1)

Degenerated primers were used to amplify a prfA specific probe from Thermus thermophilus genomic DNA by PCR. Preparation of Th. thermophilus genomic DNA and subsequent genomic PCR followed conventional protocols for mesophilic bacteria (Sambrook (2001), Molecular Cloning: A Laboratory Manual. Cold Spring Harbor Laboratory Press, NY, Vol. 1, 2, 3). A 50 mg bacterial pellet in an Eppendorf tube was resuspended in 565 μl TE buffer, 30 μl 10% SDS and 5 μl 20 mg/ml Proteinase K was added and incubated for 1 h at 37° C. Lysis was performed after addition of 100 μl 5 M NaCl and repeated uptaking and emptying the bacteria with a needle-equipped syringe by shearing forces. After addition of 80 μl of 10% Hexadecyltrimethylammoniumbromid (CTAB) in 0.7 M NaCl, 10 min incubation at 65° C. and extraction with 700 μl Chloroform/Isoamylalcohol and 5 times with 700 μl Phenol/Chloroform/Isoamylalcohol 25:24:1, genomic DNA was precipitated with Isoamylalcohol. 1 μg DNA from the genomic DNA preparation was used in a 100 μl PCR reaction containing 5 μl of each 10 μM primer, 10 μl 15 mM MgCl_{—}2, 10 μl 10× Taq Pol buffer (100 mM Tris-HCl pH 8.8, 500 mM KCl, 15 mM MgCl₂), 1 μl 1 U/μl Taq Polymerase, and cycled 30 times with 30 s at 95°, 30 s at 60° and 60 s at 72° after an initial 5 min denaturation at 95°.

With the amplified fragment, a 3.0 kbp fragment could be identified carrying the complete prfA sequence. The fragment was cloned into the plasmid pBluescript KS+ and the resulting plasmid pBlueK4b was sequenced. The sequence identified is identical to that of the literature (Ito (1997), Biochimie. 79, 287-292) and is shown in SEQ ID NO: 3. The corresponding amino acid sequence is shown in SEQ ID No: 4.

### EXAMPLE 9

### Overexpression of the Release Factor 1 of Thermus Thermophilus in E. coli and Purification of the Same

The T.th.RF1 protein was heterologous overexpressed in E. coli and purified to homogeneity as judged by SDS PAGE and Maldi mass spectroscopy. In brief, after cell lysis of RF1-overproducing E. coli cells by Lysozyme and/or Parr bomb treatment, the S100 supernatant from the ultracentrifugation was concentrated by AMS-precipitation, dialyzed against 50 mM Tris/HCl pH 7.5, 10 mM MgCl₂, 1 mM β-mercaptoethanol, 5% glycerol and used for Q-Sepharose FF (Amersham-Pharmacia) ionexchange chromatography running a gradient from 0 to 500 mM NaCl. RF1-containing fractions were pooled, 15 min at 65° C. heat-treated removing most E. coli proteins—including heterologous E. coli RF1—AMS-precipitated, dialyzed against 10 mM K-Phosphate buffer pH 6.8 and used for hydroxyapatite chromatography (Merck) running a gradient from 10 to 500 mM K-Phosphate pH 6.8. Pooled RF1-fractions were ammonium sulfate-precipitated, dialyzed against 100 mM sodium acetate pH 5.75 and used for a final ionexchange chromatography on EMD-SO₃⁻-Tentakel (Merck) running a gradient from 0 to 2 M KCl. Recovered Thermus thermophilus RF1 was ammonium sulfate-precipitated, dialyzed against 100 mM Tris/HCl pH 7.5, 100 mM KCl, 5% glycerol, and stored at −20° C. after adding an equal volume pure Glycerol.

### EXAMPLE 10

### Preparation of Polyclonal Antibodies Against the Release Factor 1 of Thermus thermophilus

Heterologous, in E. coli overexpressed and purified Thermus thermophilus RF1 was used to immunize two rabbits following the standard one month immunization-protocol at Eurogentec (Seraing, Belgium): A first immunization used the glycerinated protein mixed with incomplete Freund's adjuvant and a intradermic multisite injection at the rabbits back at day 0. Three boost immunizations at day 14, 30 and 60 followed with a small bleeding after 45 days and termination of the rabbits and final bleeding after 70 days. Vacutainer tubes were used to process blood samples and remove agglutinated blood clots.

After three boosts serum was collected, centrifuged and the polyclonal antibodies were stored at −20° C.

### EXAMPLE 11

### Preparation and Radioactive Labelling of the Puromycine-Derivatives of the Present Invention

The puromycin derivatives used herein were synthesized by standard phosphoramidite chemistry (Berg, Tymoczko, Stryer, Biochemistry, 5^{th} Edition, Freeman Co. New York, 2001 pp. 148-149) by Purimex, Staufenberg, Germany. The synthesis of the puromycin dinucleotide 5′-dC(N⁴-TEG-NH2)pPuromycin-3′ was accomplished by coupling of N⁴ alkylamino synthon (dC(N4-TEG-NH-TFA)-phosphoramidit, provided by ChemGenes, Wilmington, Mass. 01887 U.S.A., Cat-No. CLP-1329, Formula:

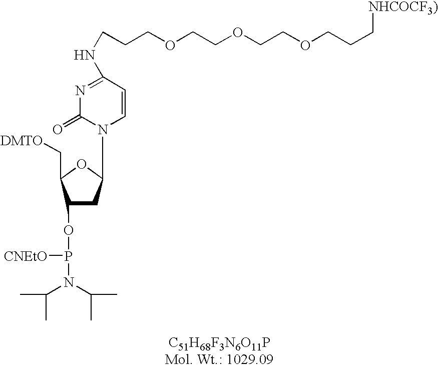

with 5′-Dimethoxytrityl-N-trifluoroacetyl-puromycin, 2′-succinyl-lcaa(long chain alkylamino)-CPG (provided by GlenResearch, Sterling, Va. 20164 U.S.A., Cat. No. 20-4040-xx, Formula:

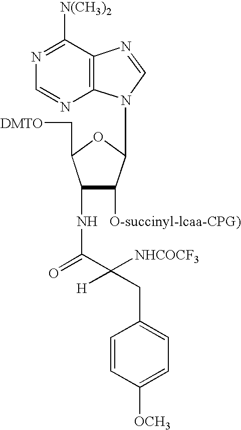

The resulting 5′-dimethoxytrityl-protected dinucleotide was cleaved from the CPG matrix by 32% ammonium hydroxide and left under this condition at 65° C. for 1 h in order to achieve total deprotection. Subsequently the dinucleotide was purified by HPLC and the trityl group was removed by treatment with 80% aqueous acetic acid solution. The final purification was achieved by two HPLC steps. The sample was concentrated by evaporation and desalted by passing through a SepPac cartridge. Concentration of the puromycin derivatives in the in vitro translation assay was 7 μM, unless otherwise indicated.

To monitor the incorporation of puromycin-derivatives during translation reactions, the used puromycin derivatives were labelled with [γ-³²P]ATP at the 5′-end by ³²P in the following way.

The reaction mixture (10 μl) contains 0.8 U/μl T4-polynucleotide kinase, 4 μM ATP, 0.2 μM [γ-³²P]ATP (10 μCi, 4950 mCi/mmol, Hartmann Analytic, Braunschweig, Germany), 4 μM puromycin-modified oligonucleotide (Purimex, Staufenberg, Germany) in T4-polynucleotid kinase buffer (70 mM Tris/HCl (pH 7.6), 10 mM MgCl₂, 5 mM DTT). Phosphorylation was carried out for 30 minutes at 37° C. Then entire volume of the reaction mixture was mixed with 6.6 μl of 100 μM puromycin derivative. The resulting solution (50 μM, 301 mCi/pmol) was used for in vitro translation experiments.

### EXAMPLE 12

### Depletion of the Release Factor 1 from E. Coli in an E. Coli Cell-Free Translation System by Precipitating and/or Crosslinking Said Release Factor 1 from E. coli with Polyclonal Antibodies Against the Release Factor 1 of Thermus thermophilus and Thereby Increasing the Incorporation of Puromycine and/or its Derivatives at the C-Terminal Nonsense Codon of the Esterase 2 (Est2) from Alicyclobacillus acidocaldarius

To further demonstrate the use of the esterase 2 of the present invention for monitoring and/or tracking the synthesis of a protein, polypeptide or peptide in a system in which the synthesis of a protein, polypeptide or peptide can occur, the following Experiment was performed.

In order to improve C-terminal incorporation of puromycin, the E. coli release factor 1 (RF1) responsible for termination at the UAA and UAG stop codons was inactivated in the in vitro translation system from E. coli by rabbit antibodies specific against Thermus thermophilus RF1. Template DNA that encoded mRNA for synthesis of the esterase 2 from Alicyclobacillus acidocaldarius (Manco (1998), Biochem. J, 332 (Pt 1), 203-212.) and ended by UAG stop codon, was used.

The C-terminal labeling of the esterase was monitored by incorporation of Biotin-Puromycin into full-length esterase protein

Further, the attachment of Biotin-Puromycin to the esterase was demonstrated by immobilization of the resulting protein-puromycin-biotin conjugate to the surface of streptavidin-coated glass plates (FIG. 11).

At position 1 of FIG. 11, 1 μL esterase purified from overexpressing E. coli strain (Manco (2000), Arch. Biochem. Biophys., 373, 182-192.) was applied to streptavidin-coated glass plates. At positions 2, 3 and 4 in vitro translation, programmed by esterase gene, was performed directly “on spot” in 1 μL translation mixture containing no puromycin derivative on the streptavidine-coated glass plates. The translation mixture without Biotin-CpPuromycin and RF1 antibody was placed on spot 2, translation mixture in the presence of Biotin-CpPuromycin on spot 3 and the translation mixture with both, Biotin-CpPuromycin and RF1 antibody on spot 4. After translation performed for 90 min. at 37° C. in a cell free translation System as described herein the unbound components were removed by rinsing the plates with tap-water. The residual activity of biotinylated esterase bound to the streptavidine coated glass plates (Greiner Bio-One, Frickenhausen, Germany) was determined by applying 2 μL solution composed of 20 mg of 2-naphthyl acetate dissolved in 1 mL of acetone and 150 mg of Fast Blue BB salt suspended in 4 mL 100 mM Tris/HCl pH 7.5, to the surface of the plate. Esterase containing spots became brown-coloured after few minutes. The reaction was stopped by rinsing the gel in tap water.

As demonstrated in FIG. 11, the control experiments on spots 1 and 2 show no esterase activity. Only very little esterase activity could be detected on spot 3 where the translation was performed in the presence of biotin-puromycin. Obviously, the competition of the puromycin-derivative with RF1 for the AUG triplet-coded A-site prevented an effective incorporation. Only after inactivation of RF1 with antibodies (FIG. 11, plate 4) significant amount of esterase has been immobilized.

The in vitro synthesis of the esterase 2 was performed according to the example 6, but in the presence of antibodies against RF1 of T. thermophilus and a biotinylated puromycin derivative. As an example, a detailed protocol of the performed in vitro translation is listed below.

Protocol for in vitro translation (RiNA GmbH Kits) in the presence of antibodies against RF1 of T. thermophilus and Puromycin derivatives:

For preparation of a 30 μl reaction mixture the following components should be combined on ice (given in order of mixing):

1. 1.5 μl of the 100 nM template plasmid
2. 2.4 μl of E-mix (red lid, supplied with the Kit)
3. 9 μl of T-mix with Leu (blue lid, supplied with the Kit)
4. 10.5 μl of S-mix (yellow lid, supplied with the Kit)
5. 2 μl of rabbit anti-RF1 antiserum
6. 4 μl of 50 μM solution of puromycin derivative (radioactively labelled)

The reaction mixture should be incubated at 37° C. for up to 2 hours with agitation (500 rpm). Aliquots can be withdrawn at any desired time intervals.

For instance, the in vitro translation system (without RF depleting agents (e.g. Antibodies against RF1 from Thermus thermophilus) and nonsense codon suppressing agents (e.g. puromycin derivatives and/or suppressor tRNAs) of the protocol listed above comprises the same ingredients as listed herein-above (Example 6).

### EXAMPLE 14

### Preparation of the Plasmid pEst2_Amb155 Comprising a cDNA Encoding an AGC¹⁵⁵→TAG¹⁵⁵-Mutated Esterase 2 (Est2) from Alicyclobacillus acidocaldarius

The ACG triplet in the est2 mRNA of Alicyclobacillus acidocaldarius esterase 2 coding for serine-155 was replaced by the RF1 stop codon UAG (amber), while the stop codon at the end of the mRNA was substituted for UGA (opal) codon that promotes RF2-dependent termination (FIG. 10A).

Therefore, site-directed mutagenesis was performed on the esterase gene (Est2) in pIVEX_Est2 (pEst2) plasmid by the overlap extension method. Two separate PCR reactions was carried out using (1) T7 promoter primer (5′-TAATACGACTCACTATAGGG-3′; SEQ ID NO: 22) and EstS115x_rev (5′-ATTCCCTCCGGCCTAGTCTCCGCCGACCGCGATGC-3′; SEQ ID NO: 23); (2) EstS115x_for (5′-CGGTCGGCGGAGACTAGGCCGGAGGGAATCTTGCC-3′; SEQ ID NO: 24) and T7 terminator primer (5′-CTAGTTATTGCTCAGCGGTG-3′; SEQ ID NO: 25). The mutated codons are bolded and the serine codon at position 155 of amino acid sequence of the esterase was changed to RF1 stop codon (TAG) mutation. The PCR fragments were fused by another PCR using T7 promoter and T7 terminator primers. The fused PCR product was digested with NcoI/SacI and ligated into NcoI/SacI digested pIVEX vector. The ligation mixture was transformed into E. coli strain XL-1 Blue. The plasmid DNA was isolated from clones and sequenced before use. The resulting plasmid pEst2_amb155 was used for in vitro translation. A map of pEst2_amb155 is shown in FIG. 12, the corresponding nucleotide sequence is shown in FIG. 13 (SEQ ID NO: 10).

The plasmid was purified as described herein above (Example 3)

### EXAMPLE 15

### Preparation of Suppressor tRNA^{Ser(CUA)}

Within the present application, the used suppressor tRNA^{SerCUA} was prepared as follows.

The gene of tRNA^{SerCUA} was constructed by PCR using primers tSer-amber1 (5′-GGAATTCTAATACGACTCACTATAGGAGAGATGCC-3′; SEQ ID NO: 26), tSer-amber2 (5′-GTCCGTTCAGCCGCTCCGGCATCTCTCCTATAGTG-3′; SEQ ID NO: 27), tSer-amber3 (5′-CTCCGGTTTTAGAGACCGGTCCGTTCAGCCGCTCC-3′; SEQ ID NO: 28), tSer-amber4 (5′-CCGGTAGAGTTGCCCCTACTCCGGTTTTAGAGACC-3′; SEQ ID NO: 29), tSer-amber5 (5′-GAGAGGGGGATTTGAACCCCCGGTAGAGTTGCCCC-3′; SEQ ID NO: 30), tSer-amber6 (5′-AAGCTTGGATGGATCACCTGGCGGAGAGAGGGGGATTTGAA C-3′; SEQ ID NO: 31). Bolded letters are T7 promoter and italic letters are the gene of tRNA^{SerCUA}. The mutated anticodon is underlined. The conditions of the performed PCR were 95° C. denaturation for 30 seconds, 50° C. annealing for 30 seconds and 72° C. polymerization for 30 seconds; 25 cycles were performed. The primer concentration was about 1 nM. DNTP concentration was 0.4 mM. The sequence of suppressor tRNA is based on a tRNA^{Ser} from E. coli (tRNA databank number DS1660) with a CUA mutation from position 34 to 36. The PCR product was cloned in a pGEM-T vector. The resulting plasmid ptSer-amber was sequenced and used as a template for the following PCR. The PCR was performed with primers tSer-amber1 and M13_rev (5′-CAGGAAACAGCTATGACC-3′; SEQ ID NO: 32). The PCR product was digested with BstNI for a CCA end and used as the template for in vitro transcription. The transcripts were purified by urea polyacrylamide gel electrophoresis and stored at −20° C.

### EXAMPLE 16

### Depletion of the Release Factor 1 from E. Coli in an E. Coli Cell-Free Translation System by Precipitating and/or Crosslinking Said Release Factor 1 from E. coli with Polyclonal Antibodies Against the Release Factor 1 of Thermus thermophilus and Thereby Increasing the Incorporation of an (Unnatural) Amino Acid Delivered by Aminoacyl Suppressor tRNA^{CUA} at an Internal Nonsense Codon of an AGC¹⁵⁵→TAG¹⁵⁵-Mutated Esterase 2 (Est2) from Alicyclobacillus acidocaldarius

To further demonstrate the use of the esterase 2 of the present invention for monitoring and/or tracking the synthesis of a protein, polypeptide or peptide in a system in which the synthesis of a protein, polypeptide or peptide can occur, additionally, the following Experiment was performed.

Using the construct of Example 14 as a template for in vitro protein synthesis, the suppression of the amber codon was studied by SDS-PAGE of the full-length esterase 2 production (FIG. 15B) and by measurement of the catalytic activity of the in vitro synthesized esterase (FIG. 15C). Translation of est2 mRNA (Ser-155) and est2 mRNA (amber-155) as measured by [¹⁴C]leucine incorporation into polypeptide chain provides a protein of 34.4 and 17.3 kDa (FIG. 15B), respectively, approximately with the same efficiency (FIG. 15A). The amber mutation in position 155 leads to complete termination and synthesis of 17.3 kDa protein void of esterase activity (FIG. 15B; lane 1 and FIG. 15C).

Addition of increasing amounts of amber suppressor tRNA^{Ser(CUA)} to the translation mixture that is programmed by est2 mRNA (amber-155) gives rise to the synthesis of the full size polypeptide chain. The required concentration of tRNA^{Ser(CUA)} for production of the full-length protein in maximal yield is about 5 μM (FIG. 16A). This value is in the range of concentrations of tRNA isoacceptors in E. coli cells (Dong (1996), J. Mol. Biol. 260, 649-663. Although, the amber suppressor Ser-tRNA^{Ser(CUA)}, with an anticodon complementary to UAG and presented in complex with EF-Tu.GTP and has the optimal prerequisites to enter the UAG-programmed A-site, it has still to compete with endogenous RF1. As demonstrated in FIGS. 16A and C this competition starts to be efficient only at μM concentration of Ser-tRNA^{Ser(CUA)}. The cellular concentration of RF1 is similar to that of tRNA isoacceptors (Dong (1996), J. Mol. Biol. 260, 649-663; Adamski (1994), J. Mol. Biol 238, 302-308.). However, during preparations of cellular extracts for in vitro translation the concentrations of all cellular components drop as compared to the situation in cytoplasm. Whereas the tRNA concentration in the in vitro translation system was adjusted by addition of bulk tRNA to 50 μM, the concentration of RF1 becomes about 50 fold lower as compared with the situation in vivo. It follows, that the average final concentration of a single aminoacyl-tRNA isoacceptor and RF1 in the in vitro translation mixture is about 1 μM and 20 nM, respectively. The need for high Ser-tRNA^{Ser(CUA)} concentrations to compete for RF1 probably reflects the different affinity for the ribosomal A-site of these alternative substrates. At concentrations higher than 1 μM, however, Ser-tRNA^{Ser(CUA)} already starts to compete also with near-cognate aminoacyl-tRNAs for codon-specified binding to the A-site and the serine becomes misincorporated into several other positions of the polypeptide chain. This leads to accumulation of errors and loss of protein functionality, i.e. inactive enzyme (FIG. 16C). At very high tRNA^{Ser(CUA)} concentrations the yield of the 17.3 and 34 kDa polypeptides drops, probably due to frameshifting and premature termination, and the [¹⁴C]leucine radioactivity becomes distributed between polypeptides of different lengths (FIG. 16A, lines 6 and 7).

Completely different is the situation in the absence of RF1 that can be efficiently deactivated by addition of antibodies against Thermus thermophilus RF1 to the E. coli in vitro translation system. Absence of RF1 leads to stimulation of UAG suppression by near-cognate endogenous tRNAs (compare FIG. 16A, line 1 and FIG. 16B, line 1). Thus, in the absence of RF1 (FIG. 16B) the synthesis of 17 kDa polypeptide substantially decreases as compared to the translation in the complete system (FIG. 16A) and only a small amount of mostly inactive, full-length protein is synthesized.

As compared to the complete system (FIG. 16A), in the absence of RF1 the concentration of tRNA^{Ser(CUA)} required to achieve full UAG suppression and synthesis of active full-length esterase 2 from est2 mRNA (amber-155) drops dramatically (FIG. 16B). Already at 24 nM tRNA^{Ser(CUA)} in the translation mixture the synthesis of the full-length (34 kDa) polypeptide becomes efficient. The esterase synthesized under these conditions is fully active. The yield of the synthesized enzyme and its activity are identical to the esterase obtained by translation of the wild-type est2 mRNA (Ser-155). The yield of active esterase remains high up to 1 μM concentration of tRNA^{Ser(CUA)} (FIG. 16C, bars 2-5). Further increase of the suppressor tRNA concentration in the translation mixture results in drop of protein production along with loss of enzymatic activity. In the high tRNA^{Ser(CUA)} concentration range there is a coincidence between the data presented in FIGS. 16A and 16B.

Thus, it was demonstrated that in the absence of RF1 the suppressor Ser-tRNA^{Ser(CUA)} is efficiently bound to the A-site of UAG-programmed ribosomes. This leads to complete suppression of UAG codon and to incorporation of the catalytically essential serine-155 into the enzyme. At high Ser-tRNA^{Ser(CUA)}.EF-Tu.GTP concentrations, the competition with other aminoacyl-tRNA.EF-Tu.GTP ternary complexes leads to misreading of near-cognate codons and results in synthesis of error prone or incomplete polypeptide chains void of enzymatic activity. Thus, the use of est2 mRNA (amber 155) as a template and the possibility to deactivate the endogenous RF1 in the in vitro translation system by RF1 antibodies permits an optimal adjustment of RF1 and tRNA^{Ser(CUA)} concentrations to achieve a complete suppression and at the same time a maximal retention of enzymatic activity of the esterase.

The kits used for in vitro translation experiments within this example were evaluation size transcription/translation kits from RiNA GmbH (Berlin, Germany) and the reaction was performed according to the manual provided by the supplier. [¹⁴C]L-Leucine (54 mCi/mmol) was added up to 160 μM along with leucine resulting in 0.5 mM total concentration. The templates pEst2 and pEst2_amb155 were added up to 5 nM concentrations. The reaction was performed at 37° C. with agitation. Aliquots, 3 μL, were withdrawn at different time intervals (up to 2 hours) and the newly synthesized protein was determined by radioactivity measurement in 10% trichloroacetic acid precipitate. Protein composition was analysed by SDS-PAG). The gels were fixed with 15% formaldehyde in 60% methanol and stained with Coomassie Blue G-250. The dried gels were exposed to an imaging plate for radioactivity analysis with the Phosphorlmager SI (Molecular Dynamics, Sunnyvale, USA).

The in vitro translations were performed according to the protocol shown in example 6 with minor modifications. As an example a detailed protocol of the in vitro translation performed in the presence of anti-RF1 (T. thermophilus) antibodies and suppressor tRNA is listed below.

Protocol for in vitro translation (RiNA GmbH Kits) in the presence of anti-RF1 (T. thermophilus) antibodies and suppressor tRNA:

For preparation of a 30 μl reaction the following components should be combined on ice (given in order of mixing):

1. 5.1 μl of 926 μM [¹⁴C]Leu (54 mCi/mmol, Amersham)
2. 1 μl 10 mM Leu (supplied with the Kit)
3. 2.4 μl of E-mix (red lid, supplied with the Kit)
4. 9 μl of T-mix without Leu (blue lid, supplied with the Kit)
5. 10.5 μl of S-mix (yellow lid, supplied with the Kit)
6. 2 μl of rabbit anti-RF1 antiserum
7. 0.5 μl of 1.52 μM tRNA^{Ser(CUA)} (can be varied in 20 fold range)
8. 0.1 μl of the 1.9 μM template plasmid

The reaction mixture should be incubated at 37° C. for up to 2 hours with agitation (500 rpm). Aliquots can be withdrawn at any desired time intervals.

For instance, the in vitro translation system (without RF depleting agents (e.g. Antibodies against RF1 from Thermus thermophilus) and nonsense codon suppressing agents (e.g. puromycin derivatives and/or suppressor tRNAs) of the protocol listed above comprises the same ingredients as listed herein-above (Example 6).

### EXAMPLE 17

### Preparation of the Plasmid pIVEX2.3d-Nox-Est2 (pNox-Est2) for Expression of a Fusion Protein Comprising NADH Oxidase (Nox) from Thermus thermophilus, Esterase 2 from Alicyclobacillus acidocaldarius and a Factor Xa-Cleavable Link

Plasmid pT7SCII, containing the gene of the esterase (Est2) was kindly provided by G. Manco, Napples, Italy (Manco (1998), Biochem. J, 332 (Pt 1), 203-212.). The gene was amplified by PCR with the primers Est2CT_for (5′-GAGCTCGGTACCATTGAGGGTCGCGGTTCCGGCGGTGGTATGGCGCTCGATC CC-3′; SEQ ID NO: 20) and Est2CT_rev (5′-GGATCCTCAGGCCAGCGC-3′; SEQ ID NO: 21). The primers create the SacI and the BamHI cleavage sites (underlined letters) upstream and downstream of the Est2, respectively. The primer Est2CT_for contains the cleavage site of protease Factor Xa coding sequence (bold letters) and the primer Est2CT_rev contains the UAG stop codon (bold letters). The PCR product was sequenced and cloned into the pIVEX2.3d vector through SacI and BamHI cleavage sites. The resulting plasmid pEst was used as a parental expression vector for further cloning.

The gene of NADH oxidase (Nox) from Thermus thermophilus was amplified from the plasmid pTthnadox310 (Lehrstuhl Biochemie, University of Bayreuth, Germany, UniPort Q60049) by PCR with the primers Nox_for (5′-CATATGGAGGCGACCCTTCCCGTTTTG-3′; SEQ ID NO: 33) and Nox_rev (5′-GAGCTCGCGCCAGAGGACCACCCGCTCCA GGG-3′; SEQ ID NO: 34). The primers introduce the NdeI and the SacI cleavage sites (underlined letters) upstream and downstream of the Nox, respectively. The PCR product was sequenced and cloned into the pEst2 vector resulting in pNox-Est2 plasmid. The plasmid can be used for in vitro translation and in vivo expression. A map of pNox-Est2 is shown in FIG. 17 and the corresponding nucleotide sequence is shown in FIG. 18 (SEQ ID NO: 11). The in vitro expression of the Nox-Est2 fusion protein was achieved with EasyXpress Protein Synthesis Kits from Qiagen (FIG. 19A). The in vivo expression of the Nox-Est2 fusion protein was achieved in E. coli strain BL21 (DE3) (FIG. 19B).

### EXAMPLE 18

### Preparation of the Plasmid pIVEX2.3d-EF-Tu-Est2 (pTu-Est2) Comprising a cDNA Encoding a Fusion Protein Comprising Elongation Factor Tu from Thermus thermophilus, Esterase 2 from Alicyclobacillus acidocaldarius and a Factor Xa Cleavable Link and Expression of the Fusion Protein

The gene of elongation factor Tu (EF-Tu) from Thermus thermophilus was amplified from the plasmid pGEM-T-EF-Tu (Lehrstuhl Biochemie, University of Bayreuth, Germany, UniPort P60338) by PCR with the primers Tu_for (5′-CCATGGCGAAGGGCGAGTTTGTTCGGACG-3′; SEQ ID NO: 35) and Tu_rev (5′-GAGCTCCAGGATCTTGGTGACGACGC CGGCGC-3′; SEQ ID NO: 36). The primers introduce the NcoI and the SacI cleavage sites (underlined letters) upstream and downstream of the EF-Tu, respectively. The PCR product was sequenced and cloned into the pEst2 vector resulting in pTu-Est2 plasmid. The plasmid can be used for in vitro translation and in vivo expression. A map of pTu-Est2 is shown in FIG. 20 and the corresponding nucleotide sequence is shown in FIG. 21 (SEQ ID NO: 12). The in vitro expression of the EF-Tu-Est2 fusion protein was achieved with EasyXpress Protein Synthesis Kits from Qiagen (FIG. 22A). The in vivo expression of the Tu-Est2 fusion protein was achieved in E. coli strain BL21 (DE3) (FIG. 22B).

### EXAMPLE 19

### Preparation of the Plasmid pIVEX2.3d-EF-Ts-Est2 (pTs-Est2) for Expression of a a Fusion Protein Comprising Elongation Factor Ts from Thermus thermophilus, Esterase 2 from Alicyclobacillus acidocaldarius and a Factor Xa Cleavable Link

The gene of elongation factor Ts (EF-Ts) from Thermus thermophilus was amplified from the plasmid pET-Ts7 (Lehrstuhl Biochemie, University of Bayreuth, Germany, UniPort P43895) by PCR with the primers Ts_for (5′-CATATGAGCCAAATGGAACTCATCAAGAAGC-3′; SEQ ID NO: 37) and Ts_rev (5′-GGTACCCGCCCCCAGCTCAAAGCGG C-3′; SEQ ID NO: 38). The primers introduce the NdeI and the KpnI cleavage sites (underlined letters) upstream and downstream of the EF-Ts, respectively. The PCR product was sequenced and cloned into the pEst2 vector resulting in pTs-Est2 plasmid. The plasmid can be used for in vitro translation and in vivo expression. A map of pTs-Est2 is shown in FIG. 23 and the corresponding nucleotide sequence is shown in FIG. 24 (SEQ ID NO: 13). The in vitro expression of the EF-Ts-Est2 fusion protein was achieved with EasyXpress Protein Synthesis Kits from Qiagen (FIG. 25A). The in vivo expression of the EF-Ts-Est2 fusion protein was achieved in E. coli strain BL21 (DE3) (FIG. 25B).

### EXAMPLE 20

### Preparation of the Plasmid pIVEX2.3d-Exportin-t-Est2 (pExp-Est2) for Expression of Human Exportin-t, Esterase 2 from Alicyclobacillus acidocaldarius and a Factor Xa Cleavable Link Fusion Protein

The gene of Exprotin-t from Human was amplified from the plasmid pExportin-t (Lehrstuhl Biochemie, University of Bayreuth, Germany, UniPort 043592) by PCR with the primers Exportin_for (5′-CCATGGATGAACAGGCTCTATTAGGGC-3′; SEQ ID NO: 39) and Exportin_rev (5′-GAGCTCGGGCTTTGCTCTCTGGAAGAACAC-3′; SEQ ID NO: 40). The primers introduce the NcoI and the SacI cleavage sites (underlined letters) upstream and downstream of the Exportin-t, respectively. The PCR product was sequenced and cloned into the pEst2 vector resulting in pExp-Est2 plasmid. The plasmid was used for in vitro translation and in vivo expression. A map of pExp-Est2 is shown in FIG. 26 and the corresponding nucleotide sequence is shown in FIG. 27 (SEQ ID NO: 14). The in vitro expression of the Exportin-t-Est2 fusion protein was achieved with EasyXpress Protein Synthesis Kits from Qiagen (FIG. 28). The yield of the fusion protein expression is low, probably due to use of heterologous system where E. coli cells (or extracts) are expected to synthesize a human protein. On the other hand the sensitive detection with esterase assay allows for optimization of conditions to achieve higher expression.

### EXAMPLE 21

### Preparation of the Plasmid pET28c-S2001-Est2 (pET-S2001-Est2) Expression of a Fusion Protein Consisting of Putative Nuclease S2001 from Sulfolobus Solfataricus, Esterase 2 from Alicyclobacillus acidocaldarius and a Factor Xa Cleavable Link

The gene of putative nuclease S2001 from Sulfolobus solfataricus was amplified from the plasmid pET28c-S2001 (Lehrstuhl Biochemie, University of Bayreuth, Germany, NCBI AAK 42190) by PCR with the primers S2001_for (5′-CCATGGGCAGCAGCCATCATCATCATCATCACAGCAGCGGCCTGGTGCCGCGC GGCAG-3′; SEQ ID NO: 41) and S2001_rev (5′GGTACCGAGCTCTAGAGTGGAACCTCC-3′; SEQ ID NO: 42). The primers introduce the NcoI and the KpnI cleavage sites (underlined letters) upstream and downstream of the nuclease S2001, respectively. The PCR product was sequenced and cloned into the pEst2 vector resulting in pS2001-Est2 plasmid. The S2001-Est2 gene was cut from pS2001-Est2 plasmid by NcoI and BamHI and cloned into pET-28c expression vector. The resulting plasmid pET-S2001-Est2 can be used for in vivo expression in E. coli strain Rosetta (DE3, pLysS). A map of pET-S2001-Est2 is shown in FIG. 29 and the corresponding nucleotide sequence is shown in FIG. 30 (SEQ ID NO: 15). The in vivo expression of the S2001-Est2 fusion protein was achieved in E. coli strain Rosetta (DE3, pLysS) (FIG. 31).

### EXAMPLE 22

### Affinity Purification of In Vivo Expressed Nox-Est2 Fusion Protein

Cells that expressed the Nox-Est fusion protein were harvested and resuspended in 10 ml of 50 mM Tris-HCl pH 7.5, 300 mM NaCl, 1 mM 2-mercaptoethanol, 1 mM EDTA, 5% glycerol, disintegrated by mild ultrasonication and centrifuged at 20,000 g for 15 minutes at 4° C. The supernatant was loaded on the 5 ml TFK-Sepharose column which was equilibrated with the above buffer and washed with the buffer to remove all cellular proteins and other components. The Nox-Est fusion protein was then eluted with 5 ml 200 mM 1,1,1-Trifluoro-3-(2-hydroxy-ethylsulfanyl)-propan-2-one (F₃C—CO—CH₂—S—CH₂—CH₂—OH) in the same buffer. The pooled fractions were dialysed against 20 mM Tris-HCl pH 7.5, 50 mM NaCl, 1 mM 2-mercaptoethanol, 1 mM EDTA, 5% Glycerol. The protein concentration was determined by Roti®-Nanoquant (Roth, Karlsruhe, Germany).

The fusion protein purified by this single step of affinity elution gave a single protein band with an apparent molecular weight of 58.5 kD based on coomassie blue stained SDS-PAGE (FIG. 32a). 1.5 mg of purified fusion protein was obtained from 1 L cell culture. The esterase-fused protein can be isolated also by cleavage with factor Xa directly from the TFK-Sepharose column. After cleavage, the esterase part of fusion protein still remains bound to TFK-Sepharose while Factor Xa and Nox are eluted from the column (FIG. 32b).

### EXAMPLE 23

### Materials Employed in this Study

Within the present application, materials were purchased as follows.

Taq polymerase was from Qiagen (Hilden, Germany), T4-DNA-Ligase from Promega (Mannheim, Germany), Factor Xa protease and restriction enzymes were from New England Biolabs (Frankfurt, Germany). Fast Blue BB Salt, p-Nitrophenyl acetate and β-Naphthyl-acetate were from Fluka (Steinheim, Germany). 5-(and 6-) Carboxy-2′,7′-dichlorofluoresceine diacetate was from Molecular probes (Eugene, USA). Other analytical grade chemicals were obtained from Roth (Karlsruhe, Germany). Radioactive [¹⁴C]leucine (54 mCi/mmol) was from Amersham, Life Sciences (Freiburg, Germany).

The present invention refers to the following nucleotide and amino acid sequences:

| SEQ ID No. 1: | |
|---|---|
| Nucleotide sequence encoding esterase 2 from | |
| Alicyclobacillus acidocaldarius: | |
| 1 | ATGCCGCTCG ATCCCGTCAT TCAGCAGGTG CTCGATCAAC TCAACCGCAT |
| 51 | GCCTGCCCCG GACTACAAAC ATCTCTCCGC CCAGCAATTT CGTTCCCAAC |
| 101 | AGTCGCTGTT TCCTCCTGTC AAGAAGGAGC CCGTGGCCGA GGTCCGAGAG |
| 151 | TTTGACATGG ATCTGCCTGG CCGCACGCTC AAGGTGCGCA TGTACCGCCC |
| 201 | GGAGGGCGTC GAACCGCCCT ACCCCGCGCT CGTGTATTAT CACGGCGGCG |
| 251 | GTTGGGTCGT CGGAGACCTC GAGACGCACG ATCCCGTCTG CCGCGTCCTC |
| 301 | GCGAAAGACG GCCGCGCGGT CGTGTTCTCC GTCGACTACC GCCTGGCGCC |
| 351 | GGAGCACAAG TTCCCTGCCG CCGTGGAAGA CGCCTACGAC GCGCTTCAGT |
| 401 | GGATCGCGGA GCGCGCAGCG GACTTTCATC TCGATCGAGC CCGCATCGCG |
| 451 | GTCGGCGGAG ACAGCGCCGG AGGGAATCTT GCCGCTGTGA CGAGCATCCT |
| 501 | TGCCAAAGAG CGCGGCGGGC CGGCCATCGC GTTCCAGCTG CTCATCTACC |
| 551 | CTTCCACGGG GTACGATCCG GCTCATCCTC CCGCATCTAT CGAAGAAAAT |
| 601 | GCGGAAGGCT ATCTCCTGAC CGGCGGCATG ATGCTCTGGT TCCGGGATCA |
| 651 | ATACTTGAAC AGCCTGGAGG AACTCACGCA TCCGTGGTTT TGACCCGTCC |
| 701 | TCTACCCGGA CTTGAGCGGC TTGCCTCCGG CGTACATCGC GACGGCGCAG |
| 751 | TACGATCGGC TGCGCGACGT CGGCAAGCTT TACGCGGAAG CGCTGAACAA |
| 801 | GGCGGGCGTC AAGGTCGAGA TCGAGAACTT CGAAGATCTG ATCCACGGAT |
| 851 | TCGCACAGTT TTACAGCCTT TCGCCTGGCG CGACGAAGGC GCTCGTCCGC |
| 901 | ATTGCGGAGA AACTTCGAGA CGCGCTGGCC TGA |
| SEQ ID No. 2: | |
| Amino acid sequence of esterase 2 from | |
| Alicyclobacillus acidocaldarius: | |
| 1 | MPLDPVIQQV LDQLNRMPAP DYKHLSAQQF RSQQSLFPPV KKEPVAEVRE |
| 51 | FDXDLPGRTL KVRXYRPEGV EPPYPALVYY HGGGWVVGDL ETHDPVCRVL |
| 101 | AKDGRAVVFS VDYRLAPEHK FPAAVEDAYD ALQWIAERAA DFHLDPARIA |
| 151 | VGGDSAGGNL AAVTSILAKE RGGPALAFQL LIYPSTGYDP AHPPASTEEN |
| 201 | AEGYLLTGGX XLWFRDQYLN SLEELTHPWF SPVLYPDLSG LPPAYIATAQ |
| 251 | YDPLRDVGKL YAEALNKAGV KVEIENFEDL IHGFAQFYSL SPGATKALVR |
| 301 | IAEKLRDALA |

With respect to SEQ ID NO: 2, it is of note that the amino acid residues indicated with X (position 53, 64, 210 and 211) are methionine residues (Met (M)) as encoded by the corresponding codon triplet “ATG” as shown in SEQ ID NO:1. The amino acid sequence corresponding to SEQ ID NO: 2 and having methionine residues (Met (M)) at amino acid position 53, 64, 210 and 211 is shown in SEQ ID NO: 62 of the sequence listing.

SEQ ID No. 3:
Nucleotide sequence encoding release factor 1
from Thermus thermophilus
atgctggacaagcttgaccgcctagaggaagagtaccgggagctggaggc
gctcctctccgacccggaggtgctgaaggacaaggggcgctaccagagcc
tctcccgccgctacgccgagatgggggaggtgatcggcctcatccgggag
taccggaaggtgctggaggacctggagcaggcggaaagccttcttgacga
ccccgagctcaaggagatggccaaggcggagcgggaggccctcctcgccc
gcaaggaggccctggagaaggagctggagcgccacctcctgcctaaggac
cccatggacgaaagggacgccatcgtagagatccgggcggggacgggagg
ggaggaggccgccctcttcgcccgcgaccttttcaacatgtacctccgct
tcgccgaggagatgggctttgagacggaggtcctggactcccaccccacg
gacctcgggggcttctccaaggtggtctttgaggtgcggggcccgggggc
ctacggcaccttcaagtacgagagcggggtccaccgggtgcaacgggtgc
ccgtcaccgagacccaggggcggatccacacctccaccgccacggtggcc
gtcctccccaaggcggaggaggaggacttcgccctcaacatggacgagat
ccgcattgacgtgatgcgggcctcggggcccggggggcagggggtgaaca
ccacggactcggcggtgcgggtggtccacctgcccacggggatcatggtc
acctgccaggactcccgcagccagatcaagaaccgggagaaggccctcat
gatcctaagaagccgtctcctggagatgaagcgggcggaggaggcggaaa
ggctccggaagacccgccttgcccagatcggcaccggggagcgctcggag
aagatccgcacctacaacttcccccagtcccgggtcacggaccaccgcat
cgggttcaccacccacgacctcgagggcgtcctctccggccacctgaccc
ccatcctggaggcgctcaagcgggccgaccaggagcgccagctcgcggcg
ctggcggaagggtga
SEQ ID No. 4:
Amino acid sequence of release factor 1 from Thermus thermophilus
MLDKLDRLEEEYRELEALLSDPEVLKDKGRYQSLSRRYAEMGEVIGLIREYRKVLEDLEQAESLLDDPELKEMAK
AEREALLARKEALEKELERHLLPKDPMDERDAIVEIRAGTGGEEAALFARDLFNMYLRFAEEMGFETEVLDSHPT
DLGGFSKVVFEVRGPGAYGTFKYESGVHRVQRVPVTETQGRIHTSTATVAVLPKAEEEDFALNMDEIRIDVMRAS
GPGGQGVNTTDSAVRVVHLPTGIMVTCQDSRSQIKNREKALMILRSRLLEMKPAEEAERLRKTRLAQTGTGERSE
KIRTYNFPQSRVTDHRIGFTTHDLEGVLSGHLTPILEALKPADQERQLAALAEG*
SEQ ID No. 5:
Nucleotide sequence encoding peptide chain release
factor 1 (RF-1)-Escherichia coli, Escherichia
coli O6, Escherichia coli O157:H7, and Shigella
flexneri.
atgaagccttctatcgttgccaaactggaagccctgcatgaacgccatga
agaagttcaggcgttgctgggtgacgcgcaaactatcgccgaccaggaac
gttttcgcgcattatcacgcgaatatgcgcagttaagtgatgtttcgcgc
tgttttaccgactggcaacaggttcaggaagatatcgaaaccgcacagat
gatgctcgatgatcctgaaatgcgtgagatggcgcaggatgaactgcgcg
aagctaaagaaaaaagcgagcaactggaacagcaattacaggttctgtta
ctgccaaaagatcctgatgacgaacgtaacgccttcctcgaagtccgagc
cggaaccggcggcgacgaagcggcgctgttcgcgggcgatctgttccgta
tgtacagccgttatgccgaagcccgccgctggcgggtagaaatcatgagc
gccagcgagggtgaacatggtggttataaagagatcatcgccaaaattag
cggtgatggtgtgtatggtcgtctgaaatttgaatccggcggtcatcgcg
tgcaacgtgttcctgctacggaatcgcagggtcgtattcatacttctgct
tgtaccgttgcggtaatgccagaactgcctgacgcagaactgccggacat
caacccagcagatttacgcattgatactttccgctcgtcaggggcgggtg
gtcagcacgttaacaccaccggttcggcaattcgtattactcacttgccg
accgggattgttgttgaatgtcaggacgaacgttcacaacataaaaacaa
agctaaagcactttctgttctcggtgctcgcatccacgctgctgaaatgg
caaaacgccaacaggccgaagcgtctacccgtcgtaacctgctggggagt
ggcgatcgcagcgaccgtaaccgtacttacaacttcccgcaggggcgcgt
taccgatcaccgcatcaacctgacgctctaccgcctggatgaagtgatgg
aaggtaagctggatatgctgattgaaccgattatccaggaacatcaggcc
gaccaactggcggcgttgtccgagcaggaataa
SEQ ID No. 6:
Amino acid sequenze of peptide chain release factor 1 (RF-1)-
Escherichia coli, Escherichia coli O6,
Escherichia coli O157:H7, and Shigella flexneri.
MKPSIVAKLEALHERHEEVQALLGDAQTIADQERFRALSREYAQLSDVSRCFTDWQQVQEDIETAQMMLDDPEMR
EMAQDELREAKEKSEQLEQQLQVLLLPKDPDDERNAFLEVRAGTGGDEAALFAGDLFRMYSRYAEARRWRVEIMS
ASEGEHGGYKEIIAKISGDGVYGRLKFESGGHRVQRVPATESQGRIHTSACTVAVMPELPDAELPDINPADLRID
TFRSSGAGGQHVNTTDSAIRITHLPTGIVVECQDERSQHKNKAKALSVLGARIHAAEMAKRQQAEASTRRNLLGS
GDRSDRNRTYNFPQGRVTDHRINLTLYRLDEVMEGKLDMLIEPIIQEHQADQLAALSEQE*
SEQ ID No. 7:
Nucleotide sequence encoding the linker
between Est2 and eGFP encoded by the plasmid
peGFP-Est2
gagctcggtaccattgagggtcgcggttccggcggtggt
SEQ ID No. 8:
Nucleotide sequence of the plasmid peGFP-Est2
cggtaccattgagggtcgcggttccggcggtggtatggcgctcgatcccgtcattcagcaggtgctcgatcaact
caaccgcatgcctgccccggactacaaacatctctccgcccagcaatttcgttcccaacagtcgctgtttcctcc
tgtcaagaaggagcccgtggccgaggtccgagagtttgacatggatctgcctggccgcacgctcaaggtgcgcat
gtaccgcccggagggcgtcgaaccgccctaccccgcgctcgtgtattatcacggcggcggttgggtcgtcggaga
cctcgagacgcacgatcccgtctgccgcgtcctcgcgaaagacggccgcgcggtcgtgttctccgtcgactaccg
cctggcgccggagcacaagttccctgccgccgtggaagacgcctacgacgcgcttcagtggatcgcggagcgcgc
agcggactttcatctcgatccagcccgcatcgcggtcggcggagacagcgccggagggaatcttgccgctgtgac
gagcatccttgccaaagagcgcggcgggccggccatcgcgttccagctgctcatctacccttccacggggtacga
tccggctcatcctcccgcatctatcgaagaaaatgcggaaggctatctcctgaccggcggcatgatgctctggtt
ccgggatcaatacttgaacagcctggaggaactcacgcatccgtggttttcaccagtcctctacccggacttgag
cggcttgcctccggcgtacatcgcgacggcgcagtacgatccgctgcgcgacgtcggcaagctttacgcggaagc
gctgaacaaggcgggcgtcaaggtcgagatcgagaacttcgaagatctgatccacggattcgcacagttttacag
cctttcgcctggcgcgacgaaggcgctcgtccgcattgcggagaaacttcgagacgcgctggcctgaggatccgg
ctgctaacaaagcccgaaaggaagctgagttggctgctgccaccgctgagcaataactagcataaccccttgggg
cctctaaacgggtcttgaggggttttttgctgaaaggaggaactatatccggatatccacaggacggqtgtggtc
gccatgatcgcgtagtcgatagtggctccaagtagcgaagcgagcaggactgggcggcggccaaagcggtcggac
agtgctccgagaacgggtgcgcatagaaattgcatcaacgcatatagcgctagcagcacgccatagtgactggcg
atgctgtcggaatggacgatatcccgcaagaggcccggcagtaccggcataaccaagcctatgcctacagcatcc
agggtgacggtgccgaggatgacgatgagcgcattgttagatttcatacacggtgcctgactgcgttagcaattt
aactgtgataaactaccgcattaaagcttatcgatgataagctgtcaaacatgagaattcgtaatcatgtcatag
ctgtttcctgtgtgaaattgttatccgctcacaattccacacaacatacgagccggaagcataaagtgtaaagcc
tggggtgcctaatgagtgagctaactcacattaattgcgttgcgctcactgcccgctttccagtcgggaaacctg
tcgtgccagctgcattaatgaatcggccaacgcgcggggagaggcggtttgcgtattgggcgctcttccgcttcc
tcgctcactgactcgctgcgctcggtcgttcggctgcggcgagcggtatcagctcactcaaaggcggtaatacgg
ttatccacagaatcaggggataacqcagqaaagaacatgtgagcaaaaggccagcaaaaggccaggaaccgtaaa
aaggccgcgttgctggcgtttttccataqgctccgcccccctgacgagcatcacaaaaatcgacgctcaagtcag
aggtggcgaaacccgacaggactataaagataccaggcgtttccccctggaagctccctcgtgcgctctcctgtt
ccgaccctgccgcttaccggatacctgtccgcctttctcccttcgggaagcgtggcgctttctcatagctcacgc
tgtaggtatctcagttcggtgtaggtcgttcgctccaagctgggctgtgtgcacgaaccccccgttcagcccgac
cgctgcgccttatccggtaactatcgtcttgagtccaacccggtaagacacgacttatcgccactggcagcagcc
actggtaacaggattagcagagcgaggtatgtaggcggtgctacagagttcttgaagtggtggcctaactacggc
tacactagaaggacagtatttggtatctgcgctctgctgaagccagttaccttcggaaaaagagttggtagctct
tgatccggcaaacaaaccaccgctggtagcggtggtttttttgtttgcaagcagcagattacgcgcagaaaaaaa
ggatctcaagaagatcctttgatcttttctacggggtctgacgctcagtggaacgaaaactcacgttaaggqatt
ttggtcatgagattatcaaaaaggatcttcacctagatccttttaaattaaaaatgaagttttaaatcaatctaa
agtatatatgagtaaacttggtctgacagttaccaatgcttaatcagtgaggcacctatctcagcgatctgtcta
tttcgttcatccatagttgcctgactccccgtcgtgtagataactacgatacgggagggcttaccatctggcccc
agtgctgcaatgataccgcgagacccacgctcaccgqctccagatttatcagcaataaaccagccagccggaagg
gccgagcgcagaagtggtcctgcaactttatccgcctccatccagtctattaattgttgccgggaagctagagta
agtagttcgccagttaatagtttgcgcaacgttgttgccattgctacaggcatcgtggtgtcacgctcgtcgttt
ggtatggcttcattcagctccggttcccaacgatcaaggcgagttacatgatcccccatgttgtgcaaaaaagcg
gttagctccttcggtcctccgatcgttgtcagaagtaagttggccgcagtgttatcactcatggttatggcagca
ctgcataattctcttactgtcatgccatccgtaagatgcttttctgtgactggtgagtactcaaccaagtcattc
tgagaatagtgtatgcggcgaccgagttgctcttgcccggcgtcaatacgggataataccgcgccacatagcaga
actttaaaagtgctcatcattqgaaaacgttcttcggggcgaaaactctcaaggatcttaccgctgttgagatcc
agttcgatgtaacccactcgtgcacccaactgatcttcagcatcttttactttcaccagcgtttctgggtgagca
aaaacaggaaggcaaaatgccgcaaaaaagggaataagggcgacacggaaatgttgaatactcatactcttcctt
tttcaatattattgaagcatttatcagggttattgtctcatgagcggatacatatttgaatgtatttagaaaaat
aaacaaataggggttccgcgcacatttccccgaaaagtgccacctgacgtctaagaaaccattattatcatgaca
ttaacctataaaaataggcgtatcacgaggccctttcgtctcgcgcgtttcggtgatgacggtgaaaacctctga
cacatgcagctcccggagacggtcacagcttgtctgtaagcggatgccgggagcagacaagcccgtcagggcgcg
tcagcgggtgttggcgggtgtcggggctggcttaactatgcggcatcagagcagattgtactgagagtgcaccat
atatgcggtgtgaaataccgcacagatgcgtaaggagaaaataccgcatcaggcgccattcgccattcaggctgc
gcaactgttgggaagggcgatcggtgcgggcctcttcgctattacgccagctggcgaaagqgggatgtgctgcaa
ggcgattaagttgggtaacgccagggttttcccagtcacgacgttgtaaaacgacggccagtgccaagcttgcat
gcaaggagatggcgcccaacagtcccccggccacggggcctgccaccatacccacgccgaaacaagcgctcatga
gcccgaagtggcgagcccgatcttccccatcggtgatgtcggcgatataggcgccagcaaccgcacctgtggcgc
cggtgatgccggccacgatgcgtccggcqtagaggatcgagatctcgatcccgcgaaattaatacgactcactat
agggagaccacaacggtttccctctagaaataattttgtttaactttaagaaggagatataccatggtgagcaag
ggcgaggagctgttcaccggggtggtgcccatcctggtcgagctggacggcgacgtaaacggccacaagttcagc
gtgtccggcgagggcgagggcgatgccacctacggcaagctgaccctgaagttcatctgcaccaccggcaagctg
cccgtgccctggcccaccctcgtgaccaccctgacctacggcgtgcagtgcttcagccgctaccccgaccacatg
aagcagcacgacttcttcaagtccgccatgcccgaaggctacgtccaggagcgcaccatcttcttcaaggacgac
ggcaactacaagacccgcgccgaggtgaagttcgagggcgacaccctggtgaaccgcatcgagctgaagggcatc
gacttcaaggaggacggcaacatcctggggcacaagctggagtacaactacaacagccacaacgtctatatcatg
gccgacaagcagaagaacggcatcaaggtgaacttcaagatccgccacaacatcgaggacggcagcgtgcagctc
gccgaccactaccagcagaacacccccatcggcgacggccccgtgctgctgcccgacaaccactacctgagcacc
cagtccgccctgagcaaagaccccaacgagaagcgcgatcacatggtcctgctggagttcgtgaccgccgccggg
atcactctcggcatggacgagctgtacaaaggcggccgcgtcgactcgagcgagct
SEQ ID No. 9:
Nucleotide sequence of the plasmid pEst2
catggcgctcgatcccgtcattcagcaggtgctcgatcaactcaaccgca
tgcctgccccggactacaaacatctctccgcccagcaatttcgttcccaa
cagtcgctgtttcctcctgtcaagaaggagcccgtggccgaggtccgaga
gtttgacatggatctgcctggccgcacgctcaaggtgcgcatgtaccgcc
cggagggcgtcgaaccgccctaccccgcgctcgtgtattatcacggcggc
ggttgggtcgtcggagacctcgagacgcacgatcccgtctgccgcgtcct
cgcgaaagacggccgcgcggtcgtgttctccgtcgactaccgcctggcgc
cggagcacaagttccctgccgccgtggaagacgcctacgacgcgcttcag
tggatcgcggagcgcgcagcggactttcatctcgatccagcccgcatcgc
ggtcggcggagacagcgccggagggaatcttgccgctgtgacgagcatcc
ttgccaaagagcgcggcgggccggccatcgcgttccagctgctcatctac
ccttccacggggtacgatccggctcatcctcccgcatctatcgaagaaaa
tgcggaaggctatctcctgaccggcggcatgatgctctggttccgggatc
aatacttgaacagcctggaggaactcacgcatccgtggttttaccccgtc
ctctacccggacttgagcggcttgcctccggcgtacatcgcgacggcgca
gtacgatccgctgcgcgacgtcggcaagctttacgcggaagcgctgaaca
aggcgggcgtcaaggtcgagatcgagaacttcgaagatctgatcctcgga
ttcgcacagttttacagcctttcgcctggcgcgacgaaggcgctcgtccg
cattgcggagaaacttcgagacgcgctggcctgagagctcccgggggggg
ttctcatcatcatcatcatcattaataaaagggcgaattccagcacactg
gcggccgttactagtggatccggctgctaacaaagcccgaaaggaagctg
agttggctgctgccaccgctgagcaataactagcataaccccttggggcc
tctaaacgggtcttgaggggttttttgctgaaaggaggaactatatccgg
atatccacaggacgggtgtggtcgccatgatcgcgtagtcgatagtggct
ccaagtagcgaagcgagcaggactgggcggcggccaaagcggtcggacag
tgctccgagaacgggtgcgcatagaaattgcatcaacgcatatagcgcta
gcagcacgccatagtgactggcgatgctgtcggaatggacgatatcccgc
aagaggcccggcagtaccggcataaccaagcctatgcctacagcatccag
ggtgacggtgccgaggatgacgatgagcgcattgttagatttcatacacg
gtgcctgactgcgttagcaatttaactgtgataaactaccgcattaaagc
ttatcgatgataagctgtcaaacatgagaattcgtaatcatgtcatagct
gtttcctgtgtgaaattgttatccgctcacaattccacacaacatacgag
ccggaagcataaagtgtaaagcctggggtgcctaatgagtgagctaactc
acattaattgcgttgcgctcactgcccgctttccagtcgggaaacctgtc
gtgccagctgcattaatgaatcggccaacgcgcggggagaggcggtttgc
gtattgggcgctcttccgcttcctcgctcactgactcgctgcgctcggtc
gttcggctgcggcgagcggtatcagctcactcaaaggcggtaatacggtt
atccacagaatcaggggataacgcaggaaagaacatgtgagcaaaaggcc
agcaaaaggccaggaaccgtaaaaaggccgcgttgctggcgtttttccat
aggctccgcccccctgacgagcatcacaaaaatcgacgctcaagtcagag
gtggcgaaacccgacaggactataaagataccaggcgtttccccctggaa
gctccctcgtgcgctctcctgttccgaccctgccgcttaccggatacctg
tccgcctttctcccttcgggaagcgtggcgctttctcatagctcacgctg
taggtatctcagttcggtgtaggtcgttcgctccaagctgggctgtgtgc
acgaaccccccgttcagcccgaccgctgcgccttatccggtaactatcgt
cttgagtccaacccggtaagacacgacttatcgccactggcagcagccac
tggtaacaggattagcagagcgaggtatgtaggcggtgctacagagttct
tgaagtggtggcctaactacggctacactagaaggacagtatttggtatc
tgcgctctgctgaagccagttaccttcggaaaaagagttggtag0tcttg
atccggcaaacaaaccaccgctggtagcggtggtttttttgtttgcaagc
agcagattacgcgcagaaaaaaaggatctcaagaagatcctttgatcttt
tctacggggtctgacgctcagtggaacgaaaactcacgttaagggatttt
ggtcatgagattatcaaaaaggatcttcacctagatccttttaaattaaa
aatgaagttttaaatcaatctaaagtatatatgagtaaacttggtctgac
agttaccaatgcttaatcagtgaggcacctatctcagcgatctgtctatt
tcgttcatccatagttgcctgactccccgtcgtgtagataactacgatac
gggagggcttaccatctggccccagtgctgcaatgataccgcgagaccca
cgctcaccggctccagatttatcagcaataaaccagccagccggaagggc
cgagcgcagaagtggtcctgcaactttatccgcctccatccagtctatta
attgttgccgggaagctagagtaagtagttcgccagttaatagtttgcgc
aacgttgttgccattgctacaggcatcgtggtgtcacgctcgtcgtttgg
tatggcttcattcagctccggttcccaacgatcaaggcgagttacatgat
cccccatgttgtgcaaaaaagcggttagctccttcggtcctccgatcgtt
gtcagaagtaagttggccgcagtgttatcactcatggttatggcagcact
gcataattctcttactgtcatgccatccgtaagatgcttttctgtgactg
gtgagtactcaaccaagtcattctgagaatagtgtatgcggcgaccgagt
tgctcttgcccggcgtcaatacgggataataccgcgccacatagcagaac
tttaaaagtgctcatcattggaaaacgttcttcggggcgaaaactctcaa
ggatcttaccgctgttgagatccagttcgatgtaacccactcgtgcaccc
aactgatcttcagcatcttttactttcaccagcgtttctgggtgagcaaa
aacaggaaggcaaaatgccgcaaaaaagggaataagggcgacacggaaat
gttgaatactcatactcttcctttttcaatattattgaagcatttatcag
ggttattgtctcatgagcggatacatatttgaatgtatttagaaaaataa
acaaataggggttccgcgcacatttccccgaaaagtgccacctgacgtct
aagaaaccattattatcatgacattaacctataaaaataggcgtatcacg
aggccctttcgtctcgcgcgtttcggtqatgacggtgaaaacctctgaca
catgcagctcccggagacggtcacagcttgtctgtaagcggatgccggga
gcagacaagcccgtcagggcgcgtcagcgggtgttggcgggtgtcggggc
tggcttaactatgcggcatcagagcagattgtactgagagtgcaccatat
atgcggtgtgaaataccgcacagatgcgtaaggagaaaataccgcatcag
gcgccattcgccattcaggctqcgcaactgttgggaagggcgatcggtgc
gggcctcttcgctattacgccagctggcgaaagggggatgtgctgcaagg
cgattaagttgggtaacgccagggttttcccagtcacgacgttgtaaaac
gacggccagtgccaagcttgcatgcaaggagatggcgcccaacagtcccc
cggccacggggcctgccaccatacccacgccgaaacaagcgctcatgagc
ccgaagtggcgagcccgatcttccccatcggtgatgtcggcgatataggc
gccagcaaccgcacctgtggcgccggtgatgccggccacgatgcgtccgg
cgtagaggatcgagatctcgatcccgcgaaattaatacgactcactatag
ggagaccacaacggtttccctctagaaataattttgtttaactttaagaa
ggagatatac

Sequences were retrieved from www.expasy.ch with entry from swiss prot database and entry from TrEMBL database.

1-32. (canceled)

33. A vector comprising a nucleic acid molecule coding for an esterase and expressing an esterase fusion protein.

34. A vector comprising a nucleic acid molecule coding for an esterase and comprising in frame at least one multiple cloning site for a part X of an esterase-X fusion protein, whereby the fusion protein to be encoded may be of the format “X-esterase” or “esterase-X”.

35. The vector according to claim 33 comprising a nucleic acid molecule coding for a fusion protein, whereby said fusion protein is in the format “X-esterase” or “esterase-X”, wherein said esterase is an esterase as which is a single chain esterase or a functional fragment thereof, and whereby said X is a protein, polypeptide or peptide selected from the group consisting of enzymes, hormones, cytokines, pheromones, growth factors, signal proteins, structural proteins, toxins, markers, reporters and the like.

36. The vector according to claim 33, comprising a nucleic acid molecule coding for a fusion protein comprising an esterase which is a single chain esterase and GFP.

37. The vector according to claim 33, wherein said fusion protein comprises a cleavable linker between said esterase and said fused protein.

38. The vector according to claim 37, wherein said cleavable linker between said esterase and said fused protein is cleavable by a factor XA protease.

39. The vector according to claim 37, wherein said cleavable linker between the esterase and said fused protein is encoded by a nucleotide sequence comprising SEQ ID NO: 7.

40. The vector according to claim 33 comprising a nucleic acid molecule having the nucleotide sequence of SEQ ID NO: 8.

41. A protein, polypeptide or peptide encoded by a vector according to claim 33.

42. A kit comprising a vector of claim 33 or a nucleic acid molecule as defined in claim 33.

43. The kit according to claim 42 for monitoring and/or tracking the synthesis of a protein, polypeptide or peptide in a cell-free translation system, wherein said monitoring and/or tracking comprises the detection of the enzymatic function or activity of said esterase.

44. The kit according to claim 43, wherein said cell-free translation system is a cell-free coupled transcription/translation system in which the synthesis of a protein, polypeptide or peptide can occur.

45. A method for monitoring and/or tracking the synthesis of a protein, polypeptide or peptide in a cell-free translation system, comprising the step of detecting the enzymatic function or activity of an esterase.

46. A method for immobilising a protein, polypeptide or peptide comprising the steps of
(a) tagging said protein, polypeptide or peptide with an esterase; and
(b) binding said esterase to an binding molecule or an esterase inhibitor,
wherein said esterase binding molecule or inhibitor is immobilized on a solid substrate.

47. The method according to claim 46 further comprising the steps of
(c) cleaving said protein, polypeptide or peptide from said esterase; and
(d) recovering a purified fraction of said protein, polypeptide or peptide.

48. A method for the purification of a protein, polypeptide or peptide comprising the steps of:
(a) expressing in vitro said protein, polypeptide or peptide in a format of an esterase fusion construct or tagging said protein, polypeptide or peptide with an esterase;
(b) immobilizing said esterase fusion construct or said esterase tag protein, polypeptide or peptide according to the step provided in claim 46(b);
(c) cleaving said protein, polypeptide or peptide from said esterase; and
(d) recovering a purified fraction of said protein, polypeptide or peptide.

49. The method according to claim 46, wherein said tagging of said protein, polypeptide or peptide with said esterase is effected through the production of a fusion protein by a vector comprising a nucleic acid molecule coding for an esterase and expressing an esterase fusion protein in a cell-free coupled transcription/translation system in which the synthesis of said protein, polypeptide or peptide can occur.

50. The method according to claim 46, wherein said esterase inhibitor is a trifluoromethyl ketone.

51. The method according to claim 47, wherein said cleaving is effected by a factor XA protease.

52. The method according to claim 45, wherein said esterase is a single chain esterase.

53. The method according to claim 45 wherein said protein, polypeptide or peptide to be synthesized is selected from the group consisting of enzymes, hormones, cytokines, pheromones, growth factors, signal proteins, structural proteins, toxins, markers, reporters and the like.

54-56. (canceled)
